(19) United States
(12) Patent Application Publication
(10) Pub. No.: US 2009/0100532 A1
(43) Pub. Date: Apr. 16, 2009

(54) TRANSGENIC MICE EXPRESSING HYPERSENSITIVE NICOTINIC RECEPTORS

(75) Inventors: Ryan Drenan, Pasadena, CA (US); Henry A. Lester, South Pasadena, CA (US)

Correspondence Address: DLA PIPER LLP (US), 4365 EXECUTIVE DRIVE, SUITE 1100, SAN DIEGO, CA, 92121-2133, US

(73) Assignee: CALIFORNIA INSTITUTE OF TECHNOLOGY, Pasedena, CA (US)

(21) Appl. No.: 12/237,310

(22) Filed: Sep. 24, 2008

Related U.S. Application Data
(60) Provisional application No. 60/995,138, filed on Sep. 25, 2007.

(51) Int. Cl. A01K 67/027

(52) U.S. Cl. 800/3; 800/13; 800/15; 800/16; 800/19; 800/18; 800/25

(57) ABSTRACT

Provided herein are transgenic non-human animals having a transgene encoding a variant nicotinic acetylcholine receptor (nAChR) subunit, wherein the variant nAChR subunit is selected from the group consisting of α6, α5, and β2. The transgenic animals display a modified phenotype that includes nicotinic hypersensitivity. Also provided are methods of generating the invention transgenic animals. Further provided are methods for screening a candidate agent for the ability to modulate nicotine-mediated behavior in the invention transgenic animals.

### RELATED APPLICATIONS

This application claims the benefit of priority under 35 U.S.C. § 119(e) of U.S. Provisional Application Ser. No. 60/995,138, filed Sep. 25, 2007, the entire content of which is incorporated herein by reference.

### GRANT INFORMATION

This invention was made with government support from the National Institutes of Health, Grant Nos. DA19375 and DA21492. The United States government has certain rights in this invention.

### FIELD OF THE INVENTION

The present invention relates generally to animal model systems useful for examining and manipulating neurobehaviors mediated by nicotine. More specifically, the invention relates to transgenic animals having a variant nicotinic acetylcholine receptor subunit gene resulting in nicotine hypersensitivity.

### BACKGROUND

The identification of the relevant nicotinic acetylcholine receptors (nAChRs) involved in 1) normal dopamine (DA) transmission, 2) disorders of the DA system such as schizophrenia, Parkinson's disease, and ADHD, and 3) nicotine dependence, is of importance in the study of these receptors. In general, the role of a particular neurotransmitter receptor is assessed with both loss-of-function and gain-of-function experiments. Experiments with pharmacological blockade and loss-of-function mutations identify the actions for which a receptor of interest is necessary. Experiments with selective pharmacological activation, or with gain-of-function/“sensitizing” mutations, define the actions for which activation of the receptor is sufficient. Information about α6* (* indicates that other subunits may be present in the pentameric receptor) nAChRs has been confined to determinations of “necessity” at present.

α6* nicotinic acetylcholine receptors (α6* nAChRs) are highly and selectively expressed in dopaminergic neurons, with additional functional expression in locus coeruleus and retinal ganglion cells. α6* nAChRs in midbrain DA areas are selectively inhibited by the marine cone snail peptide α-conotoxin MII (αCtxMII). Immunoprecipitation and αCtxMII binding studies demonstrated that α6β2β3* and α6α4β2β3* pentamers are the predominant α6* nAChRs in mammalian striatum. α6β2* receptors account for 30% of nicotine-stimulated DA release in striatum. β3 subunits are encoded by a gene adjacent to α6, are usually co-expressed with α6, and are essential for α6* nAChR biogenesis and function. α6* receptors exhibit the highest known sensitivity to nicotine and ACh in functional measurements on native receptors, yet function poorly in heterologous expression systems. As a result, there has been little progress in defining selective agonists for α6* nAChRs, or on other positive functional measurements. Furthermore, in midbrain DA neurons, studies of somatodendritic α6* receptors are complicated by the presence of α4β2* (non-α6), and selective antagonists of α4β2* (non-α6) have not been identified.

The role of a particular neuronal cell type is also assessed with loss-of-function and gain-of-function experiments. Many experiments with selective destruction of DA neurons show that activity of such neurons is necessary for reinforcement of natural and artificial rewards. These cells exhibit tonic and phasic firing patterns, where phasic or “burst” firing carries salient information thought to predict imminent reward status. Pedunculopontine tegmentum (PPTg) and laterodorsal tegmentum (LDTg) fibers provide a cholinergic drive that strongly regulates DA neuron excitability and the transition to burst firing. Midbrain nAChRs in three locations respond to mesopontine-derived ACh: 1) α7* nAChRs on glutamatergic terminals from cerebral cortex, 2) α4β2* nAChRs on GABAergic terminals and cell bodies, and 3) α4* and α6* somato-dendritic nAChRs on DA neurons. Nicotine interferes with normal cholinergic transmission to DA neurons, in part, by modifying the weights of these various nAChR synapses. For example, nicotine at concentrations found in the CSF of smokers preferentially desensitizes α4β2* nAChRs regulating midbrain GABA release, yet still permits α7* nAChR-regulated glutamate release. This produces both disinhibition and direct excitation of DA neurons, increasing the probability of a switch to burst firing.

Nicotine also exaggerates the action of endogenous ACh in regulating DA release in striatum. Striatal cholinergic interneurons continually release ACh that activates nAChRs, which maintains background DA levels during tonic firing of midbrain DA neurons. However, DA release in response to burst firing of DA neurons is facilitated by a reduction in nAChR activity. This reduction occurs during presentation of salient information, and possibly during nAChR desensitization in response to tobacco smoking. α6* receptors, due to their high sensitivity and their selective expression in DA cell bodies and presynaptic terminals, are probably key players in cholinergic control of DA release. It was therefore reasoned that “sensitization” of these receptors would both 1) amplify the role of α6* nAChRs in cholinergic control of DA transmission, 2) allow for selective pharmacological stimulation of these neurons. This approach would complement previous experiments using α6 knock-out mice and α6* pharmacological blockade, demonstrating behavioral and physiological responses for which α6* nAChRs are sufficient.

The approach described herein involves the selective sensitization or activation of α6* nAChRs by endogenous ACh or low doses of nicotine. Accordingly, a bacterial artificial chromosome (BAC) transgene was introduced into the mouse germline with a mutant copy of the mouse α6 nAChR subunit gene that rendered mutant α6* channels “hypersensitive” to endogenous ACh or exogenous nicotine. As demonstrated herein, DA neuron excitability and DA release is greatly augmented in these mice, which exhibit behavioral phenotypes consistent with increased DA neuron firing and/or DA release. These studies improve our knowledge of cholinergic regulation of the midbrain DA system and of α6* nAChR biology, and have implications for disorders involving excess DA.

### SUMMARY OF THE INVENTION

In one embodiment of the present invention, there is provided a transgenic non-human animal having a transgene encoding a variant nicotinic acetylcholine receptor (nAChR) subunit, wherein the variant nAChR subunit is selected from the group consisting of α6, α5, and β2, and wherein further the expression of the variant results in an animal that displays a modified phenotype compared to a wild type animal. In particular embodiments, the modified phenotype includes nicotinic hypersensitivity. In certain embodiments the variant subunit comprises a mutation in the M2 transmembrane region of the nAChR subunit.

In another embodiment of the present invention, there is provided a transgenic mouse having a transgene encoding a variant nicotinic acetylcholine receptor (nAChR) subunit, wherein the variant nAChR subunit is selected from the group consisting of α6, α5, and β2, and wherein further the expression of the variant results in a mouse that displays a modified phenotype compared to a wild type mouse. In particular embodiments, the modified phenotype includes nicotinic hypersensitivity. In certain embodiments the variant subunit comprises a mutation in the M2 transmembrane region of the nAChR subunit.

In another embodiment of the present invention, there are provided methods of generating a transgenic mouse having a transgene encoding a variant nicotinic acetylcholine receptor (nAChR) subunit, wherein the method includes microinjecting a transgene into a mouse single-cell fertilized egg, wherein the variant incudes a mutation in the M2 transmembrane region of an nAChR subunit selected from the group consisting of α6, α5, and β2; transferring the microinjected egg cell into a pseudopregnant mouse surrogate; and identifying mice comprising the transgene from mice born from the surrogate. In some embodiments, the transgene is contained in a bacterial artificial chromosome.

In yet another embodiment of the present invention, there are provided methods for screening a candidate agent for the ability to modulate nicotine-mediated behavior in the transgenic animal having a transgene encoding a variant nicotinic acetylcholine receptor (nAChR) subunit, wherein the variant nAChR subunit is selected from the group consisting of α6, α5, and β2, and wherein further the expression of the variant results in an animal that displays a modified phenotype compared to a wild type animal. The method includes administering to a first transgenic animal a candidate agent, and comparing nicotine-mediated behavior of the first transgenic animal to nicotine-mediated behavior of a second transgenic animal not administered the candidate agent. A difference in nicotine-mediated behavior in the first transgenic animal administered the candidate agent compared to the second transgenic animal not administered the candidate agent is indicative of a candidate agent that modifies nicotine-mediated behavior. In some embodiments, the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.

In yet another embodiment of the present invention, there are provided methods for screening for candidate agent that modulates a nicotinic acetylcholine receptor (nAChR) subunit, in which the method includes administering a candidate agent to a transgenic animal of the invention and determining the effect of the agent upon a cellular or molecular process associated with nicotinic hypersensitivity compared to an effect of the agent administered to a non-transgenic animal. In such methods, a difference in effect is indicative of an agent that modulates nicotine hypersensitivity.

In still another embodiment of the present invention, there are provided methods for screening for candidate agent that modulates nicotine hypersensitivity, in which the method includes administering a candidate agent to a transgenic animal containing a variant nicotinic acetylcholine receptor (nAChR) subunit and comparing nicotine-mediated behavior of the first transgenic animal to nicotine-mediated behavior of a non-transgenic littermate animal administered the same dose of the candidate agent, wherein a difference in effect is indicative of an agent that modulates the nicotinic acetylcholine receptor (nAChR) subunit. In some embodiments, the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.

### BRIEF DESCRIPTION OF THE DRAWINGS

FIG. 1 shows plots of the locomotor activity of α6^{L′S} or WT mice under various conditions. Raw locomotor activity data (# ambulations per 15 min period) are shown in FIG. 1A. Total locomotor activity from “lights on” and “lights off” periods indicated in FIG. 1A are shown 1B for WT and α6_{L9′S} mice. FIGS. 1C and 1D show the locomotor activity of α6_{L9′S} and WT cagemates that were removed from their home cage and immediately placed in a fresh cage. Raw locomotor activity data (# ambulations per min) are reported in FIG. 1C. Total locomotor activity from (1C) for t=0-15 min or t=16-30 min is shown FIG. 1D. FIG. 1E shows the nicotine-stimulated locomotor activity of WT and α6^{L9′S} mice, line 2 and 5. FIG. 1F shows the dose-response relationship for nicotine-stimulated locomotor activity in WT and α6_{L9′S} mice. FIG. 1G shows the locomotor activity following saline or nicotine injection in WT and α6_{L9′S} mice. Total raw locomotor activity data (# ambulations per min, 30 min total) are reported. FIG. 1H shows the nicotine-stimulated locomotor activation in α6_{L9′S} mice and WT mice pre-injected with saline, mecamylamine, SCH23390, sulpiride, prazosin, or yohimbine. FIG. 1I shows normalized activity of α6_{L9′S} mice injected with saline once daily for three consecutive days followed by nicotine once daily for six consecutive days.

FIGS. 2A-2C show nicotine-stimulated DA release from synaptosomes of striatum of α6_{L9′S} transgenic mice and WT. FIGS. 2D-2F show hypersensitive DA release in olfactory tubercle of α6_{L9′S} transgenic mice and WT mice. FIGS. 2G and 2H show quantification of hypersensitive DA release in striatum (G) and olfactory tubercle (H). Average nicotine EC₅₀ values for each concentration response curve from (A-F) are shown. FIGS. 2I-K show the results of a nicotine-stimulated GABA release assay in striatum in the presence and absence of αCtxMII.

FIG. 3A shows the structure of TC 2429, an alpha6* selective nicotinic agonist. FIGS. 3B-D show DA release stimulated with a range of nicotine concentrations, and total (FIG. 3B) release, as well as αCtxMII-sensitive (FIG. 3C) and αCtxMII-resistant (FIG. 3D) components for TC 2429 are shown for each genotype. FIG. 3E shows the structure of TC 2403. FIGS. 3F-H shows an α4*-selective nicotinic agonist modestly activates striatal α6* nAChRs in α6_{L9′S} mice; total DA release (FIG. 3F), as well as αCtxMII-sensitive (FIG. 3G) and αCtxMII-resistant (FIG. 3H) components are shown for TC 2403. FIGS. 3I and 3J show saline-normalized locomotor activity in α6_{L9′S} and WT mice administered TC 2429 (FIG. 3I) or TC 2403 (FIG. 3J). Locomotor activity for each mouse was normalized to saline control injections in the same mouse. FIG. 3K shows a plot depicting the correlation between α6*-mediated DA release and locomotor activity, for nicotine, TC 2429, and TC 2403.

FIG. 4A shows a diagram of coronal sections from mouse brain containing ventral tegmental area. FIG. 4B shows an image of tyrosine hydroxylase (TH) staining of DA neurons in coronal slices. FIG. 4C shows a micrograph of VTA neuron studied with local nicotine application. FIG. 4D shows patch-clamp recordings taken from VTA DA neurons in coronal slices from α6_{L9′S} and WT mice. FIG. 4E shows the concentration-response relationship for hypersensitive nicotinic responses in WT and α6_{L9′S} mouse lines. FIG. 4F shows nicotine induced currents in VTA DA neurons in α6_{L9′S} slices in the presence and absence of αCtxMII or dihydro-β-erythroidine (DHβE). FIG. 4G shows currents in VTA DA neurons in α6_{L9′S} slices when the indicated drug was applied followed by activation of nAChRs with local application of 1 μM nicotine. FIG. 4H shows a representative firing response is shown for a dopamine neuron from each genotype. FIG. 4I shows a quantification of firing responses in WT and α6_{L9′S} VTA DA neurons.

FIG. 5 shows spontaneous α6 channel activity in α6_{L9′S} VTA DA neurons. FIG. 5A shows current fluctuations in voltage-clamp recordings from VTA DA neurons from WT or α6_{L9′S} mice. FIGS. 5B and 5C shows voltage clamped recordings from VTA DA neurons from α6_{L9′S} (FIG. 5B) and WT (FIG. 5C) in the presence and absence αCtxMII. FIG. 5D shows RMS noise values for voltage clamp recordings from VTA DA neurons in the presence and absence of αCtxMII.

FIG. 6A shows VTA DA neurons expressing α6_{L9′S} nAChRs, identified by the presence of large (>100 pA) inward nicotinic currents. Action potential firing in response to QP is shown for a representative neuron expressing (panel i; n=10/10) or not expressing (panel ii; n=⅘) α6_{L9′S} receptors. FIG. 6B shows the identification of substantia nigra (SN) DA and GABA neurons. A diagram of coronal sections (bregrna −3.1 mm) from mouse brain containing SN pars compacta (SNc) and pars reticulata (SNr) is shown. SN DA versus GABA neurons were identified by i) location: SNc contains DA neurons whereas SNr is largely GABAergic; ii) DA neurons express hyperpolarization-activated cation current (Iₕ); iii) DA neurons exhibit pacemaker firing (1-5 Hz) whereas GABA neurons fire at >10 Hz; iv) DA neurons have broad spikes (>2 ms) whereas GABA neurons have narrow (<1 ms) spikes. FIG. 6C shows recordings of neurons in slices from WT, α6_{L9′S} and α4_{L9′A} mice, patch clamped in whole cell configuration. FIG. 6D shows the quantification of current amplitudes from FIG. 6C.

FIG. 7A shows a diagram of coronal sections containing locus coeruleus. FIG. 7B shows tyrosine hydroxylase (TH) staining of NE neurons in coronal slices from an α6_{L9′S} mouse. FIG. 7C shows the electrophysiological identification of LC neurons. LC neurons are identified by i) pacemaker firing at 1-2 Hz, ii) lack of Iₕ, and iii) lack of membrane potential “sag” for hyperpolarizing current pulses (responses shown for injection of −80, −40, and +20 pA). FIG. 7D shows patch-clamp recordings taken from LC neurons in coronal slices from WT, α4_{L9′A}, and α6_{L9′S} mice. FIG. 7E shows the concentration-response relationship for hypersensitive nicotinic responses in WT, α4_{L9′A} and α6_{L9′S} mice. FIG. 7F shows the inhibition of hypersensitive α6_{L9′S} nAChRs by αCtxMII in LC neurons in α6_{L9′S} slices. FIG. 7G shows action potential firing in noradrenergic neuron firing in the presence of moderate nicotine concentration in α6_{L9′S} mice and WT mice. FIG. 7H shows the quantification of firing responses in WT and α6_{L9′S} LC neurons.

FIGS. 8A-D show schematics for baseline and nicotine-induced activation of DA neurons in WT mice and α6_{L9′S} mice.

FIG. 9A shows a schematic describing the BAC recombineering to generate α6_{L9′S} targeting vector. FIG. 9B shows the genomic DNA sequence of WT and transgenic mice (SEQ ID NO'S 1 & 18). FIG. 9C shows an analysis of genomic DNA samples to identify non-transgenic and transgenic mice. FIG. 9D shows a plot of the transgene copy number analysis by real time quantitative PCR. FIG. 9E shows the characterization of α6 (WT and L9′S) mRNA expression by RT-PCR in line 2 and 5 whole-brain samples. FIG. 9F shows images of brain sections from WT and α6^{L9′S} mice labeled with [¹²⁵I]-αCtxMII. Representative sections through striatum (bregma+1.4 mm), optic tract/hippocampus (bregma −2.8 mm), and superior colliculus (bregma −3.9 mm) are shown. FIG. 9G shows a quantitative analysis of α6* receptor expression in brain regions labeled with [¹²⁵I]-epibatidine in the presence or absence of competing, unlabeled αCtxMII. Raw binding values for αCtxMII-resistant receptors are shown in the upper panel. αCtxMII-sensitive receptors (predominantly α6*) are expressed as the percent of total epibatidine binding. ST-striatum, OT-olfactory tubercle, SC-superior colliculus, TH-thalamus.

FIG. 10 shows ⁸⁶Rb⁺ efflux from synaptosomes from superior colliculi from WT and α6_{L9′S} mice (line 2 and 5) were dissected and a crude synaptosomal pellet was prepared. αCtxMII-sensitive (center panel) and resistant (right panel) components are shown for each mouse line.

FIG. 11 shows a plot of the nicotinic current density in WT and α6_{L9′S} VTA neurons.

FIG. 12 shows a plot of average RMS noise values for voltage clamp recordings from α6_{L9′S} VTA DA neurons under control conditions and in the presence of the indicated drug is shown.

FIG. 13A shows a concentration-response relationship for hypersensitive nicotinic responses in SNc neurons from WT and α6_{L9′S} mouse lines. FIG. 13B shows a plot of the quantification of firing responses in WT and α6_{L9′S} SNc DA neurons. FIG. 13C shows a plot of the RMS noise values for voltage clamp recordings from SNc DA neurons in the presence and absence of αCtxMII.

FIG. 14 shows representative whole-cell patch clamp recordings of VTA dopamine neurons from WT (top panel) or α6_{L9′S} (bottom panel) mice during baseline firing (control) and firing during bath application of sulpiride.

FIG. 15 shows an analysis of striatal cholinergic interneurons from WT and α6_{L9′S} mice. FIG. 15A shows the identification of striatal cholinergic interneurons. Large, aspiny neurons from dorsal striatum often fired spontaneously (i; scale bars: 80 mV, 4 s) but in an irregular fashion, with periods of regular firing, bursts, and pauses in firing accompanied with a hyperpolarization in the resting membrane potential. Cholinergic cells expressed prominent Iₕ currents (ii; 400 pA, 0.4 s; Vm stepped from −60 mV to −70, −80, −90, −100, −110, and −120) and exhibited a substantial sag in the membrane potential in response to hyperpolarizing current pulses (iii; scale bars: 80 mV, 0.4 s; current (pA) pulses: +20, 0, −20, −40, −60, −80, −100). FIG. 15B shows the pontaneous firing properties of WT and α6_{L9′S} cholinergic interneurons. FIG. 15C shows the resting membrane potential in WT and α6_{L9′S} cholinergic interneurons. FIG. 15D shows the Iₕ currents in striatal cholinergic interneurons in α6_{L9′S} mice and WT mice. FIG. 15E shows whole-cell recordings of striatal cholinergic interneurons from WT, line 2, and line 5. Membrane potential, including the sag in the membrane potential that is dependent on Iₕ currents, was measured in response to the indicated hyperpolarizing current injections. Voltage sag for each current injection was defined as the difference between the peak hyperpolarized potential and the steady state potential at the end of the current pulse (inset).

FIG. 16 shows a schematic depicting the nicotine EC₅₀ values for functional nAChRs in α6_{L9′S} mice from various neurons.

FIG. 17 shows the α6 and β3 nAChR constructs (SEQ ID NO'S19 & 20) used in Example 2.

FIG. 18A shows an image of fluorescently labeled β3 subunits expressed on the cell surface in Xenopus oocytes. FIG. 18B shows representative voltage-clamped responses from Xenopus oocytes expressing α3β4, α3β4β3, or α3β4β3-YFP. FIG. 18C shows representative voltage-clamped responses from oocytes expressing α3β4, α3β4β3^{V13S}, or α3β4β3-YFP^{V13S}.

FIGS. 19A and B show concentration response relationships for WT and fluorescently labeled β-containing receptors.

FIG. 20A shows a FRET schematic diagram of the nicotinic receptor. FIGS. 20B-G show images and plots of the FRET analysis.

FIG. 21 shows the fluorescently-labeled, α4-containing nicotinic receptor pentamers assayed and plots of the FRET analysis.

FIG. 22 shows the FRET analysis of α6 subunits assembling with α4 and 12 nAChR subunits.

FIG. 23 shows the fluorescently-labeled, α6-containing nicotinic receptor pentamers assayed and plots of the FRET analysis.

FIG. 24 shows the β3 and α6 subunit stoichiomentry analyzed by FRET.

FIG. 25A shows a representative response of voltage-clamped cells stimulated with 1 uM ACh for 500 ms and expressing the indicated nAChR subtype. FIG. 25B shows the quantification of electrophysiology data in 25A.

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, an and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention relates to transgenic animals expressing a hypersensitive nicotinic acetylcholine receptor. Nicotinic acetylcholine receptors (nAChRs) are multi-subunit proteins, are members of a ligand-gated receptor family and mediate rapid synaptic transmission in the central and peripheral nervous systems. Like the other type of acetylcholine receptors, the opening of nAChR channels is triggered by the endogenous neurotransmitter acetylcholine (ACh), but they are also opened by nicotine. nAChRs are made up of five receptor subunits, arranged symmetrically around a central pore. A number of nAChR subunits have been identified (e.g., α2-10, and β2-4) and have been well-characterized in the art with respect to, for example, amino acid and nucleotide sequence. The topology of nAChR subunits has been determined and includes multiple membrane-spanning domains (termed M1, M2, M3, and M4).

Thus, in one embodiment of the present invention, there is provided a transgenic non-human animal having a transgene encoding a variant nicotinic acetylcholine receptor (nAChR) subunit, wherein the variant nAChR subunit is selected from the group consisting of α6, α5, β2, and β3 and wherein further the expression of the variant results in an animal that displays a modified phenotype compared to a wild type animal. In some embodiments, the nAChR subunit is selected from the group consisting of α6, α5, and β2. In particular embodiments, the modified phenotype includes nicotinic hypersensitivity.

In particular embodiments, the nAChR subunit is the α6 subunit. α6 nicotinic ACh receptor subunits are expressed in several catecholaminergic nuclei in the central nervous system, in the locus coeruleus, and dopaminergic neurons located in the substantia nigra and ventral tegmental area. α6 nicotinic ACh receptor subunits are also expressed in retinal ganglion cells. Ligand-binding studies using the β6-specific probe α-conotoxin Mul suggest that many α6* (* indicates that other subunits may be present in the receptor) receptors are located on presynaptic terminals in the superior colliculus and striatum. Indeed, this binding activity disappears in the brains of α6 knockout mice. This strikingly specific expression pattern could indicate a unique function for α6* receptors. Accordingly, α6* receptors are candidate drug targets for diseases or disorders such as Parkinson's disease or nicotine addiction.

In another embodiment of the present invention, there is provided a transgenic mouse having a transgene encoding a variant nicotinic acetylcholine receptor (nAChR) subunit, wherein the variant comprises a mutation in the M2 transmembrane region of an nAChR subunit selected from the group consisting of α6, α5, β2, and β3, and wherein further the expression of the variant results in a mouse that displays a modified phenotype compared to a wild type mouse. In particular embodiments, the mutation is at any position of the M2 transmembrane region of the nicotinic acetylcholine receptor subunit, and further wherein the mutation renders the receptor hypersensitive In some embodiments, the nAChR subunit is selected from the group consisting of α6, α5, and β2. In particular embodiments, the nAChR subunit is the α6 subunit.

Native α6* receptors are readily studied using synaptosome preparations from brain tissue. Indeed, α-conotoxin MII-sensitive receptors are pharmacologically and stoichiometrically distinct from α-conotoxin MII-resistant receptors in mediating [³H]dopamine release from striatal synaptosomes. Recent studies using α4 and β3 knockout mice demonstrate the existence of functional α6β2, α6β2β3, α6α4β2, and α6α4β2β3 receptors (Salminen et al., Mol. Pharmacol. 71:1563:71, 2007). It is noteworthy that native α6α4β2β3 receptors have the highest affinity (EC50=0.23±0.08 μM) for nicotine of any nicotinic receptor reported to date. Because nicotine is likely to be present at concentrations ≦0.5 μM in the cerebrospinal fluid of smokers, only those receptors with the highest affinity for nicotine, including some α4* and α6* receptors, are likely to be important in nicotine addiction.

In certain embodiments, the variant subunit comprises a mutation in the M2 transmembrane region of the nAChR subunit. In particular embodiments, the mutation is at position 9′ of the M2 transmembrane region of the nAChR subunit. In certain embodiments, the mutation at position 9′ is a leucine to serine substitution. In other embodiments, the mutation at position 9′ is a leucine to alanine mutation. In still other embodiments, the mutation is at position 13′ or 16′ of the M2 transmembrane region of the nAChR subunit.

In certain embodiments, the variant nAChR subunit comprises a detectable label. The detectable label may be incorporated into the subunit by methods well-known in the art. Such labels may be, for example, a fluorescent label such as green fluorescent protein (GFP).

In particular embodiments, the modified phenotype of the transgenic animal includes nicotinic hypersensitivity. In certain embodiments, transgenic animal displays psychomotor stimulation by low doses of nicotine, a lack of locomotor sensitization upon repeated activation of nAChRs, a lack of locomotor tolerance upon repeated activation of nAChRs, spontaneous home cage locomotor hyperactivity, or a combination thereof.

Various methods to make the transgenic animals of the subject invention can be employed. The particular method used herein is described in Chen, et al. (2000), Nature 403(6769): 557-60, herein incorporated by reference in its entirety. In addition and generally speaking, three such methods may be employed. In one such method, an embryo at the pronuclear stage (a “one cell embryo”) is harvested from a female and the transgene is microinjected into the embryo, in which case the transgene will be chromosomally integrated into both the germ cells and somatic cells of the resulting mature animal.

In another method, embryonic stem cells are isolated and the transgene incorporated therein by electroporation, plasmid transfection or microinjection, followed by reintroduction of the stem cells into the embryo where they colonize and contribute to the germ line. Methods for microinjection of mammalian species is described in U.S. Pat. No. 4,873,191. An exemplary knock-in mouse having a mutation in the α4 nAChR subunit is described in U.S. Pat. No. 6,753,456, the contents of which are incorporated herein by reference.

In yet another such method, embryonic cells are infected with a retrovirus containing the transgene whereby the germ cells of the embryo have the transgene chromosomally integrated therein. When the animals to be made transgenic are avian, because avian fertilized ova generally go through cell division for the first twenty hours in the oviduct, microinjection into the pronucleus of the fertilized egg is problematic due to the inaccessibility of the pronucleus. Therefore, of the methods to make transgenic animals described generally above, retrovirus infection is preferred for avian species, for example as described in U.S. Pat. No. 5,162,215. If microinjection is to be used with avian species, however, a recently published procedure by Love et al., (Biotechnology, Jan. 12, 1994) can be utilized whereby the embryo is obtained from a sacrificed hen approximately two and one-half hours after the laying of the previous laid egg, the transgene is microinjected into the cytoplasm of the germinal disc and the embryo is cultured in a host shell until maturity. When the animals to be made transgenic are bovine or porcine, microinjection can be hampered by the opacity of the ova thereby making the nuclei difficult to identify by traditional differential interference-contrast microscopy. To overcome this problem, the ova can first be centrifuged to segregate the pronuclei for better visualization. In the microinjection method useful in the practice of the subject invention, the transgene is digested and purified free from any vector DNA e.g. by gel electrophoresis. It is preferred that the transgene include an operatively associated promoter which interacts with cellular proteins involved in transcription, ultimately resulting in constitutive expression. Promoters useful in this regard include those from cytomegalovirus (CMV), Moloney leukemia virus (MLV), and herpes virus, as well as those from the genes encoding metallothionin, skeletal actin, P-enolpyruvate carboxylase (PEPCK), phosphoglycerate (PGK), DHFR, and thymidine kinase. Promoters for viral long terminal repeats (LTRs) such as Rous Sarcoma Virus can also be employed. When the animals to be made transgenic are avian, preferred promoters include those for the chicken P-globin gene, chicken lysozyme gene, and avian leukosis virus. Constructs useful in plasmid transfection of embryonic stem cells will employ additional regulatory elements well known in the art such as enhancer elements to stimulate transcription, splice acceptors, termination and polyadenylation signals, and ribosome binding sites to permit translation.

Retroviral infection can also be used to introduce transgene into a non-human animal, as described above. The developing non-human embryo can be cultured in vitro to the blastocyst stage. During this time, the blastomeres can be targets for retro viral infection (Jaenich, R., Proc. Natl. Acad. Sci. USA 73:1260-1264, 1976). Efficient infection of the blastomeres is obtained by enzymatic treatment to remove the zona pellucida (Hogan, et al. (1986) in Manipulating the Mouse Embryo, Cold Spring Harbor Laboratory Press, Cold Spring Harbor, N.Y.). The viral vector system used to introduce the transgene is typically a replication-defective retro virus carrying the transgene (Jahner, et al., Proc. Natl. Acad. Sci. USA 82:6927-6931, 1985; Van der Putten, et al., Proc. Natl. Acad. Sci. USA 82:6148-6152, 1985). Transfection is easily and efficiently obtained by culturing the blastomeres on a monolayer of virus-producing cells (Van der Putten, supra; Stewart, et al., EMBO J. 6:383-388, 1987). Alternatively, infection can be performed at a later stage. Virus or virus-producing cells can be injected into the blastocoele (D. Jahner et al., Nature 298:623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retroviral infection of the midgestation embryo (D. Jahner et al., supra).

A third type of target cell for transgene introduction is the embryonic stem cell (ES). ES cells are obtained from pre-implantation embryos cultured in vitro and fused with embryos (M. J. Evans et al. Nature 292:154-156, 1981; M. O. Bradley et al., Nature 309: 255-258, 1984; Gossler, et al., Proc. Natl. Acad. Sci. USA 83: 9065-9069, 1986; and Robertson et al., Nature 322:445-448, 1986). Transgenes can be efficiently introduced into the ES cells by DNA transfection or by retro virus-mediated transduction. Such transformed ES cells can thereafter be combined with blastocysts from a nonhuman animal. The ES cells thereafter colonize the embryo and contribute to the germ line of the resulting chimeric animal. (For review see Jaenisch, R., Science 240: 1468-1474, 1988).

In an example of an α4 knockin mouse, a 129/SvJ α4 genomic clone containing exon 5 and the L9′S mutation was inserted into pKO Scrambler V907 (Lexicon-Genetics, The Woodlands, Tex.). A neomycin resistance cassette, with a phosphoglycerate kinase promoter and polyadenylation signal and flanked by loxp sites, was inserted 163 bp downstream from exon 5 for positive selection. The diphtheria toxin A chain gene with the RNA polymerase II promoter was inserted to provide negative selection for random insertion. Embryonic stem (ES) cells were electroporated with the linearized construct and screened by Southern blot; the wild-type (WT) gene contains a 9.7-kb, BamFH-BamHI fragment, and the mutant gene contains a 7.7-kb, BanHI-EcoRI fragment. The loxP-flanked neomycin resistance cassette was deleted in some ES cells by transfection with a cytomegalovirus-Cre plasmid; this deletion leaves only the 34 bp of one loxP site in the intron. Two lines of mice were generated by injection of mutated ES cells into C57BL/6 blastocysts, one with the neo cassette still present (neo intact) and another with the neo cassette deleted (neo deleted). The presence of the mutation was confirmed by sequence analysis of PCR-amplified gene segments.

In some embodiments of the invention there are provided methods of generating a transgenic mouse having a transgene encoding a variant nicotinic acetylcholine receptor (nAChR) subunit. In some embodiments, the method includes microinjecting a transgene into a mouse single-cell fertilized egg, wherein the variant comprises a mutation in the M2 transmembrane region of an nAChR subunit selected from the group consisting of α6, α5, and β2; transferring the microinjected egg cell into a pseudopregnant mouse surrogate; and identifying mice comprising the transgene from mice born from the surrogate. In certain embodiments, the transgene is contained in a bacterial artificial chromosome (BAC). In particular embodiments, the mutation results in an amino acid substitution at position 9′ of the M2 transmembrane region of the α6 nicotinic acetylcholine receptor subunit as compared to a wild-type mouse. In certain embodiments, the amino acid substitution is a leucine-to-serine substitution or a leucine-to-alanine substitution at position 9′ in the M2 transmembrane region. In some embodiments, the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.

In another embodiment of the invention, there are provided methods for producing a transgenic mouse having a modified behavior compared to a normal mouse. The method includes introducing a transgene having a nucleotide sequence encoding a selectable marker and encoding a leucine-to-serine mutation or a leucine-to-alanine mutation at position 9′ in the M2 transmembrane region of an α6 nicotinic receptor subunit gene into a mouse embryonic stem cell and introducing a mouse embryonic stem cell comprising the transgene in its genome into a mouse embryo. The embryo is transplanted into a pseudopregnant mouse and allowed to develop to term. Transgenic mice whose genome comprises a mutation of the endogenous α6 nicotinic receptor subunit gene, wherein the mutation results in the mouse having a modified behavior compared to a wild type mouse are identified.

The “non-human animals” of the invention are murine, bovine, ovine, porcine, avian, and piscine animals (e.g., mouse, rat, cow, pig, sheep, chicken, turkey), for example. In certain embodiments, the non-human animals are non-human mammals. The “transgenic non-human animals” of the invention are produced by introducing “transgenes” into the germline of the non-human animal. Embryonal target cells at various developmental stages can be used to introduce transgenes. Different methods are used depending on the stage of development of the embryonal target cell. The zygote is the best target for microinjection. The use of zygotes as a target for gene transfer has a major advantage in that in most cases the injected DNA will be incorporated into the host gene before the first cleavage (Brinster et al., Proc. Natl. Acad. Sci. USA 82:4438-4442, 1985). As a consequence, all cells of the transgenic non-human animal will carry the incorporated transgene. This will in general also be reflected in the efficient transmission of the transgene to offspring of the founder since 50% of the germ cells will harbor the transgene.

The term “transgenic” is used to describe an animal which includes exogenous genetic material within the genome of all of its cells.

In certain embodiments, the transgenic animal is heterozygous for the variant nicotinic acetylcholine receptor subunit gene. In other embodiments, the transgenic animal is homozygous for the variant nicotinic acetylcholine receptor subunit gene.

“Transformed” means a cell into which (or into an ancestor of which) has been introduced, by means of recombinant nucleic acid techniques, a heterologous nucleic acid molecule. “Heterologous” refers to a nucleic acid sequence that either originates from another species or is modified from either its original form or the form primarily expressed in the cell.

“Transgene” means any piece of DNA which is inserted by artifice into a cell, and becomes part of the genome of the organism (i.e., either stably integrated or as a stable extrachromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence which is transcribed into DNA and then incorporated into the genome. The transgenes of the invention include DNA sequences that include antisense, dominant negative encoding polynucleotides, which may be expressed in a transgenic non-human animal. As used herein, the term “transgenic” includes any transgenic technology familiar to those in the art which can produce an organism carrying an introduced transgene.

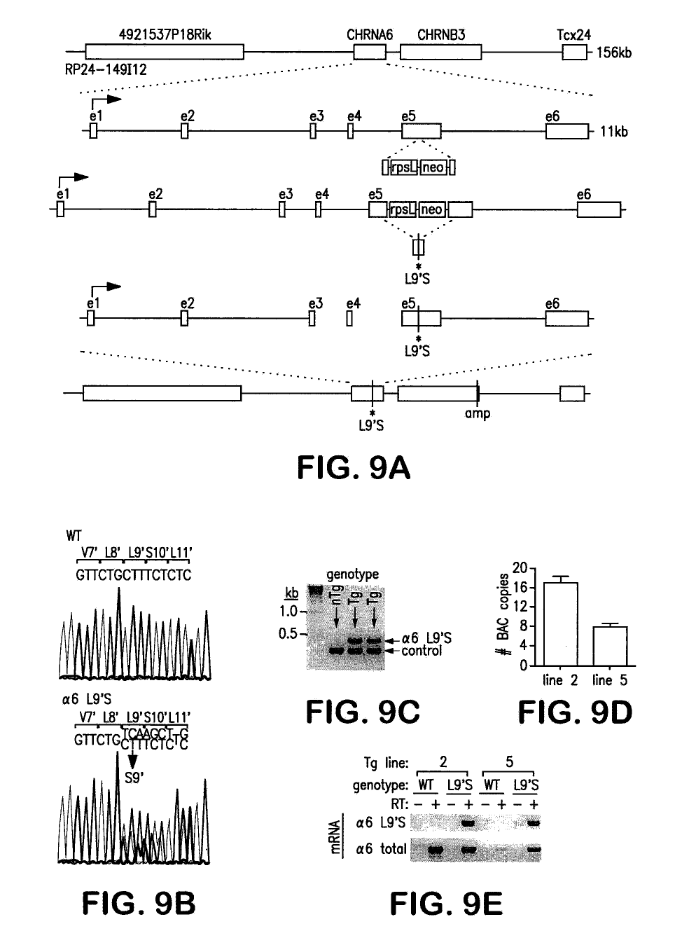

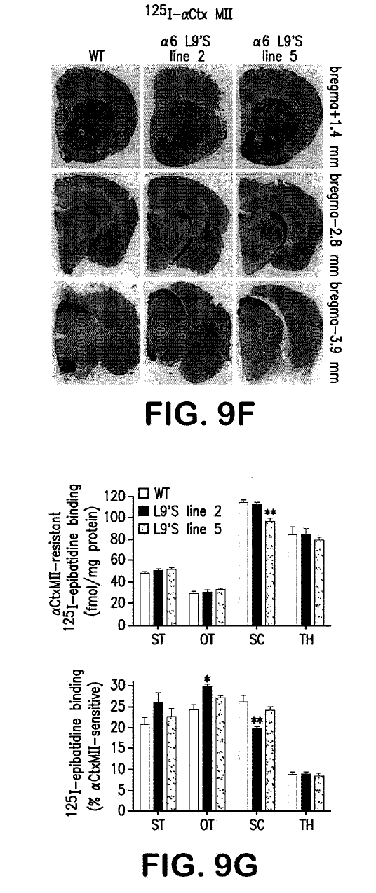

An example of a transgene used in the generation of a transgenic α6 nAChR function in the present Examples is described in Example 1 and see FIG. 9. Thus, in another embodiment, the invention provides a BAC clone containing a transgene wherein the α6 gene contains a Leu9′Ser mutation. The α6 genomic clone can also contain part or all of exon 5.

After an embryo has been microinjected, colonized with transfected embryonic stem cells or infected with a retrovirus containing the transgene (except for practice of the subject invention in avian species which is addressed elsewhere herein) the embryo is implanted into the oviduct of a pseudopregnant female. The consequent progeny are tested for incorporation of the transgene by Southern blot analysis of blood samples using transgene specific probes. PCR is particularly useful in this regard. Positive progeny (G0) are crossbred to produce offspring (G1) which are analyzed for transgene expression by Northern blot analysis of tissue samples. To be able to distinguish expression of like-species transgenes from expression of the animals endogenous nAChR subunit gene(s), a marker gene fragment can be included in the construct in the 3′ untranslated region of the transgene and the Northern probe designed to probe for the marker gene fragment. The levels of nAChR subunit can also be measured in the transgenic animal to establish appropriate expression.

The expression of transgenes can also be assessed by the incorporation of reporter molecules. Reporter molecules, which confer a detectable phenotype on a cell, are well known in the art and include, for example, fluorescent polypeptides such as green fluorescent protein, cyan fluorescent protein, red fluorescent protein, or enhanced forms thereof, an antibiotic resistance polypeptide such as puromycin N-acetyltransferase, hygromycin B phosphotransferase, neomycin (aminoglycoside) phosphotransferase, and the Sh ble gene product; a cell surface protein marker such as the cell surface protein marker neural cell adhesion molecule (N-CAM); an enzyme such as beta-lactamase, chloramphenicol acetyltransferase, adenosine deaminase, aminoglycoside phosphotransferase, dihydrofolate reductase, thymidine kinase, luciferase or xanthine guanine phosphoribosyltransferase polypeptide; or a peptide tag such as a c-myc peptide, a polyhistidine, a FLAG epitope, or any ligand (or cognate receptor), including any peptide epitope (or antibody, or antigen binding fragment thereof, that specifically binds the epitope; see, for example, Hopp et al., BioTechnology 6:1204 (1988); U.S. Pat. No. 5,011,912, each of which is incorporated herein by reference). Expression of a reporter molecule can be detected using the appropriate instrumentation or reagent, for example, by detecting fluorescence of a green fluorescent protein or light emission upon addition of luciferin to a luciferase reporter molecule, or by detecting binding of nickel ion to a polypeptide containing a polyhistidine tag. Similarly, expression of a selectable marker such as an antibiotic can be detected by identifying the presence of cells growing under the selective conditions.

A reporter molecule also can provide a means of isolating or selecting a cell expressing the reporter molecule. For example, the reporter molecule can be a polypeptide that is expressed on a cell surface and that contains an operatively linked c-myc epitope; an anti-c-myc epitope antibody can be immobilized on a solid matrix; and cells, some of which express the tagged polypeptide, can be contacted with the matrix under conditions that allow selective binding of the antibody to the epitope. Unbound cells can be removed by washing the matrix, and bound cells, which express the reporter molecule, can be eluted and collected. Methods for detecting such reporter molecules and for isolating the molecules, or cells expressing the molecules, are well known to those in the art (see, for example, Hopp et al., supra, 1988; U.S. Pat. No. 5,011,912). As indicated above, a convenient means of isolating and selecting cells expressing a reporter molecule is provided by using a reporter molecule that confers antibiotic resistance, and isolating cells that grow in the presence of the particular antibiotic.

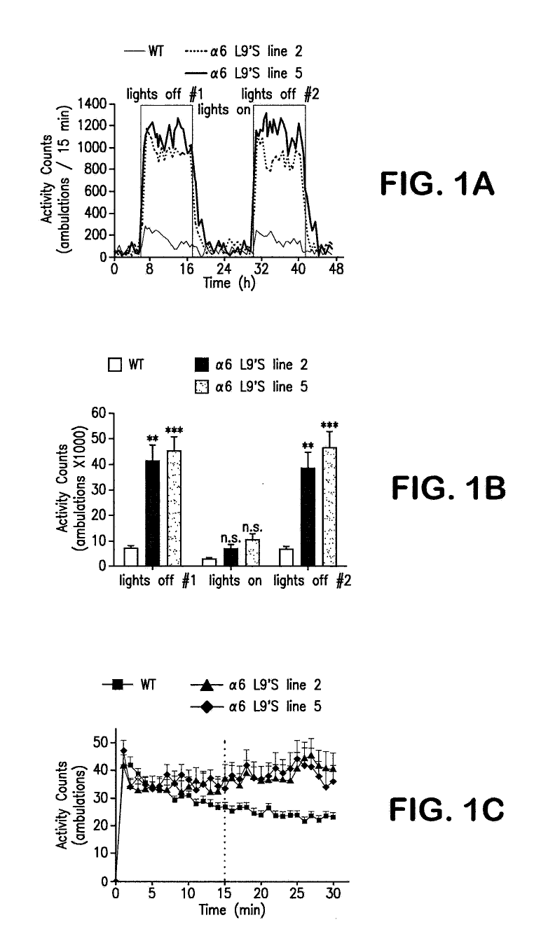

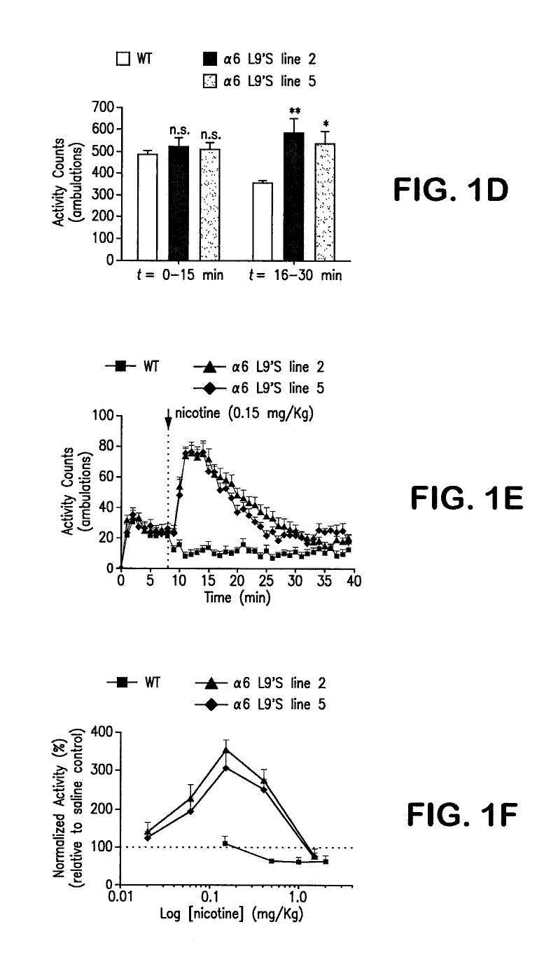

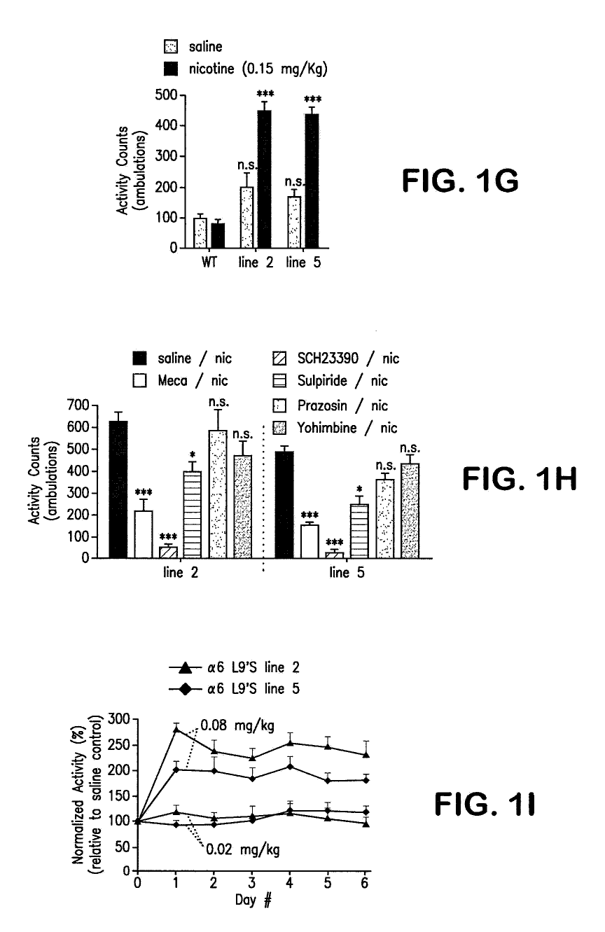

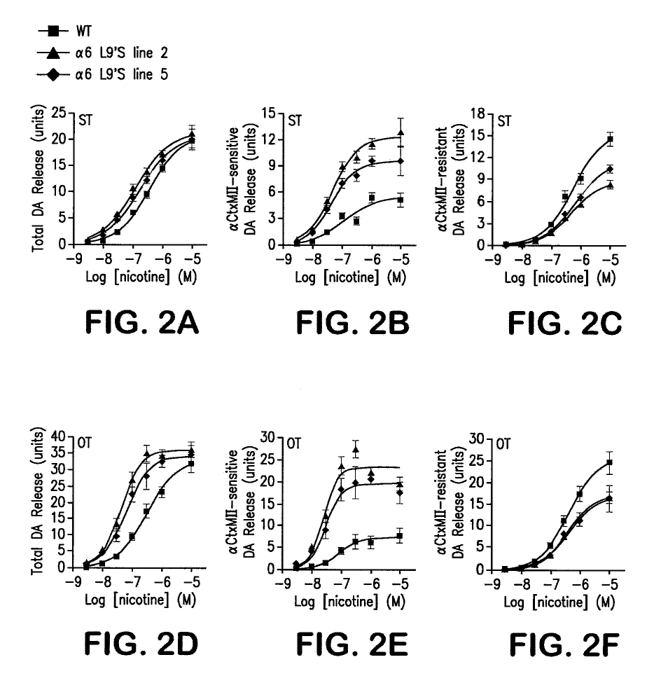

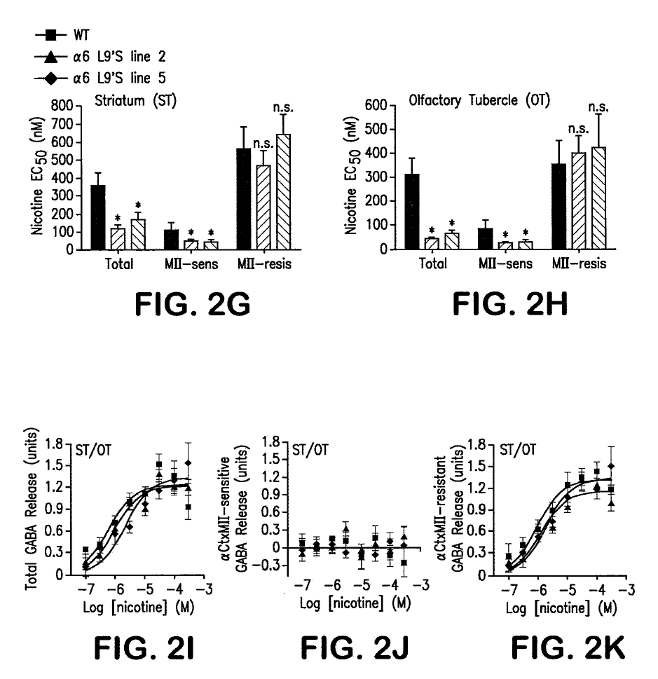

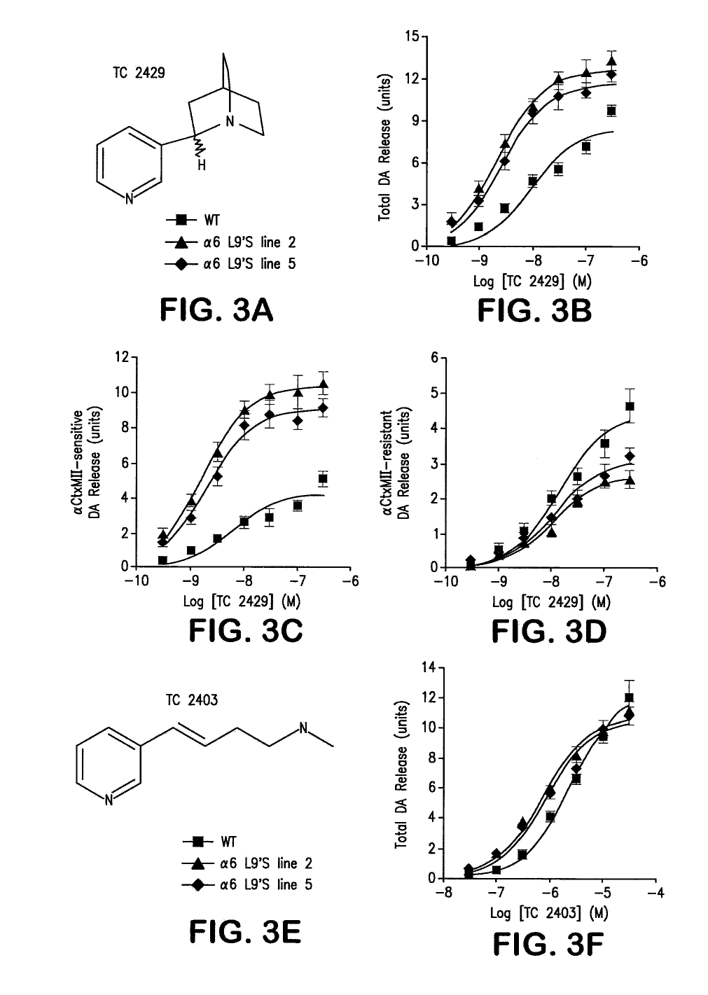

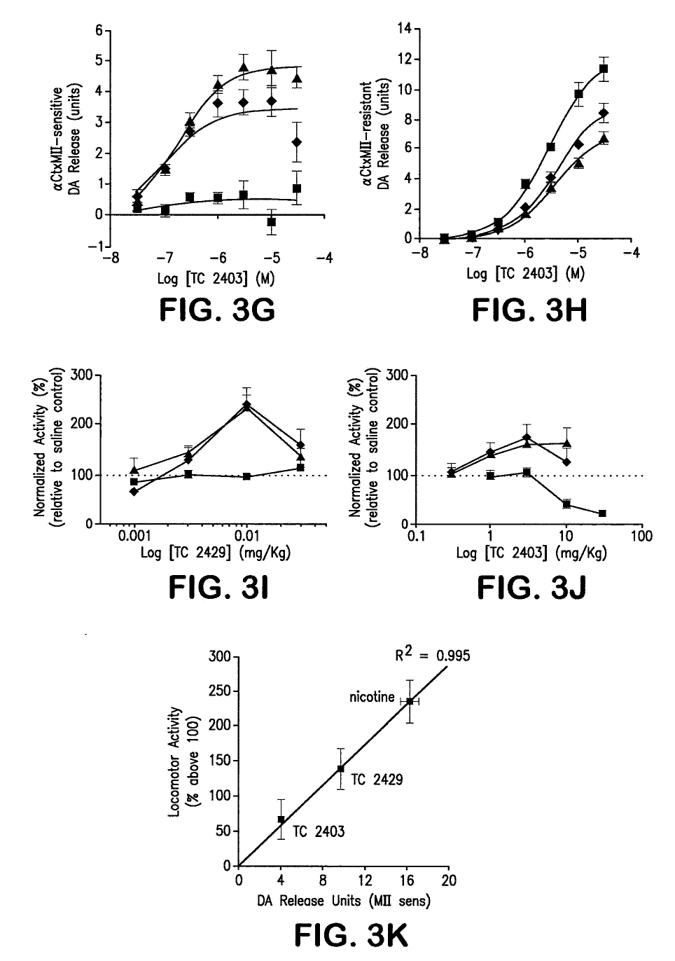

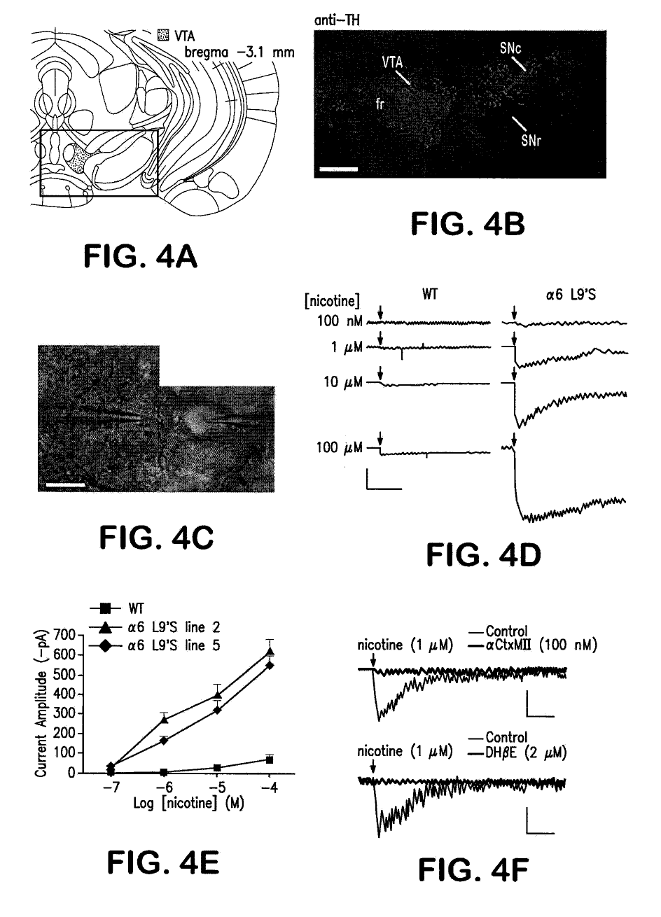

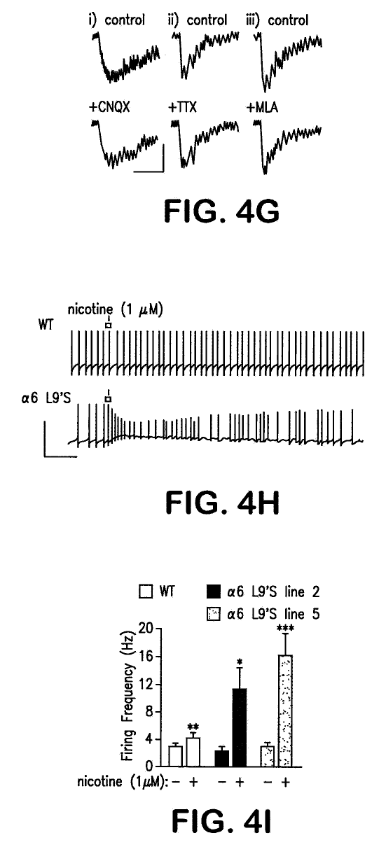

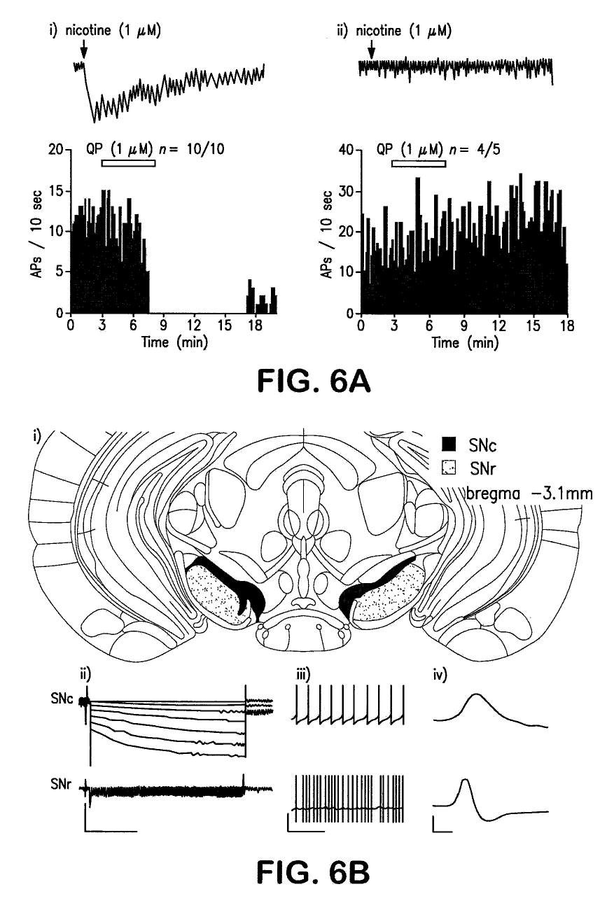

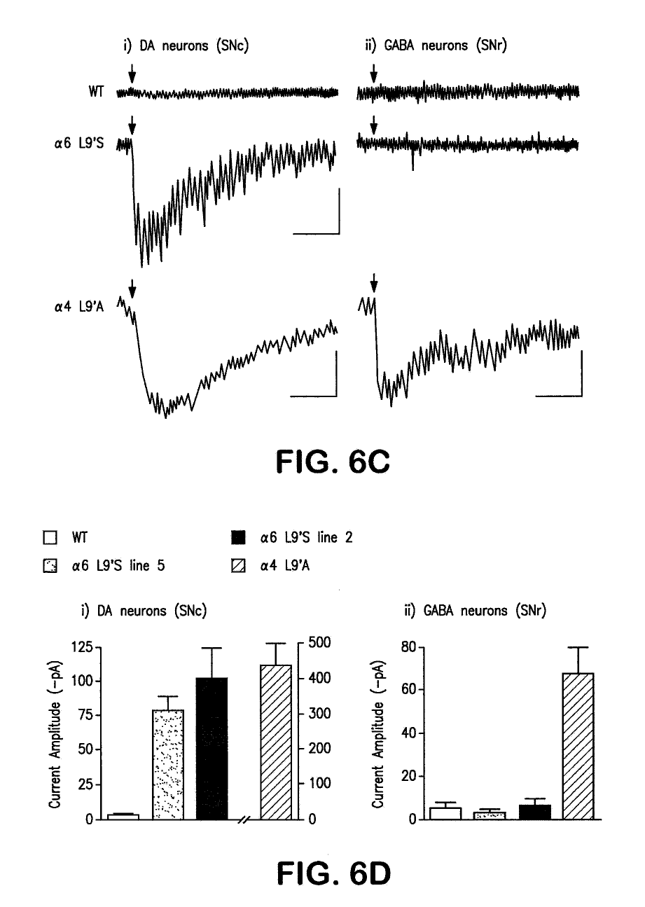

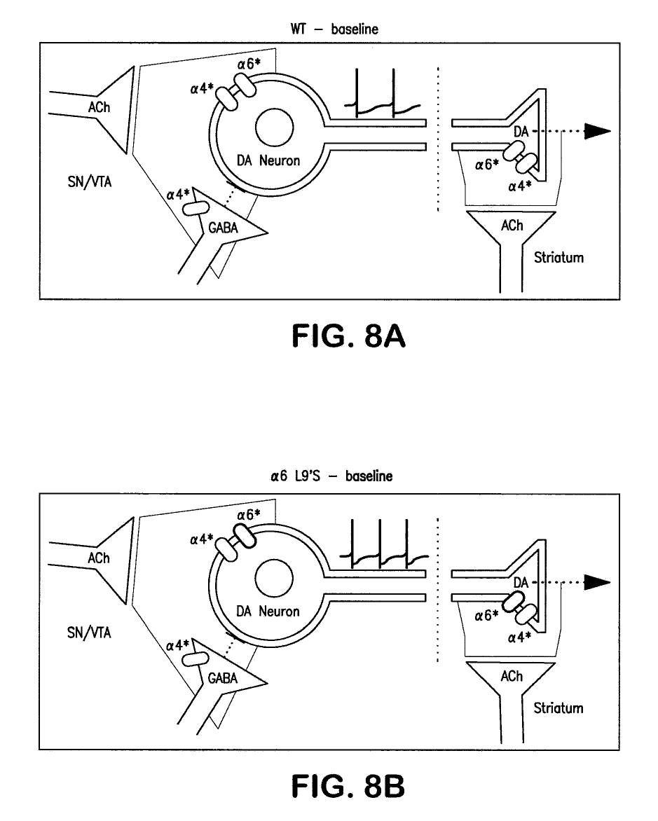

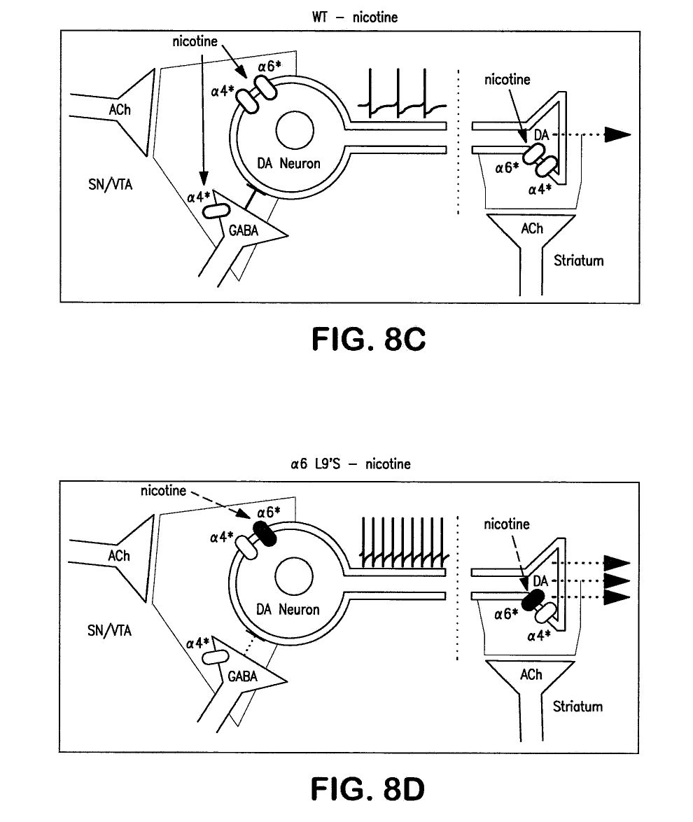

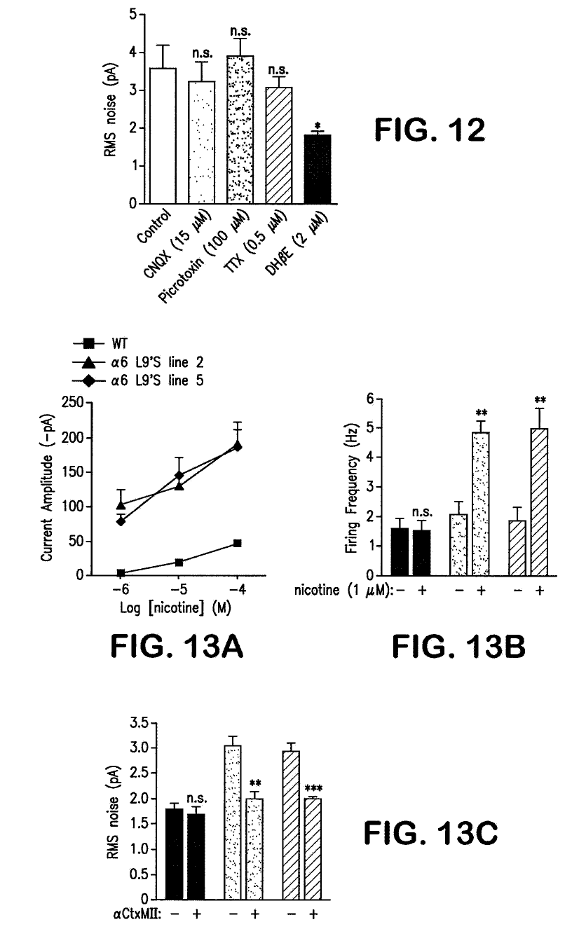

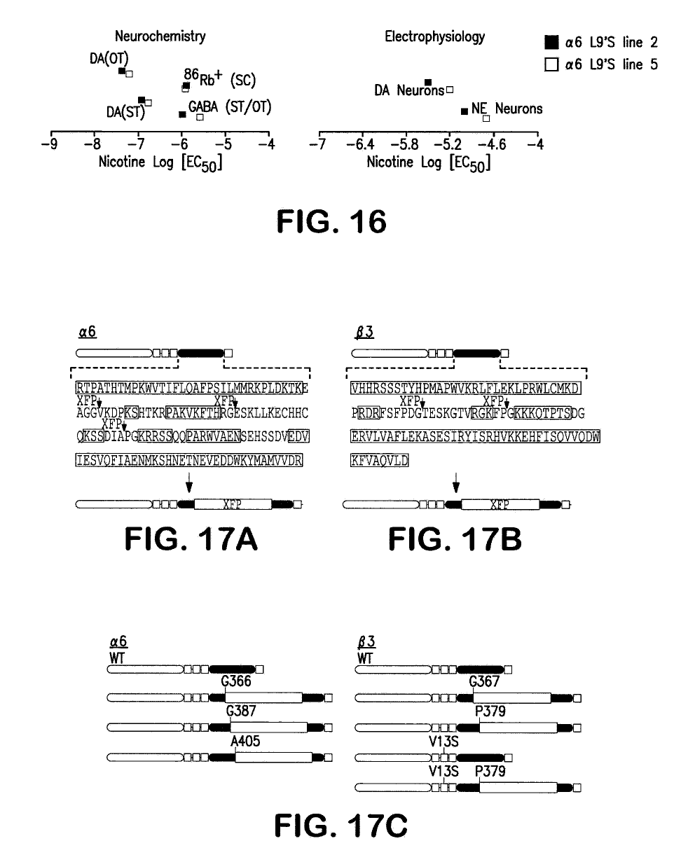

Reported herein are several new aspects of α6* nAChR biology and its role in cholinergic regulation of DA transmission. Also, shown are the behavioral effects of specifically activating DA neurons (FIG. 1). The electrophysiology and neurochemistry experiments reveal a major role for α6* nAChRs in regulating both DA neuron firing (FIGS. 4 and 13) and synaptic release of DA in the striatum (FIGS. 2 and 3). The finding that α6* nAChRs are largely excluded from midbrain GABA neurons (FIG. 6) and striatal GABAergic terminals (FIG. 2I-K), in stark contrast to α4β2* nAChRs (Nashmi et al., J Neurosci 27:8202-18, 2007), is supported behavioral data suggesting unchecked DA transmission in α6_{L9′S} mutant mice. Proposed herein is a model in which specific functional expression of α6^{L9′S} nAChRs in DA neurons renders these cells selectively hypersensitive (FIG. 16) to activation by endogenous ACh (FIG. 8B) or exogenous nicotine (FIG. 8D). This likely reflects that in WT mice, or in humans, high-affinity α6* nAChRs are specifically poised to modulate the activity of monoamine neurotransmitters such as DA. These studies provide long-sought sufficiency data for α6* nAChRs, complementing studies utilizing α6* loss-of-function mutations and pharmacological blockade.

This present study achieves specific pharmacological activation of DA neurons in vivo. By analyzing concentration-response relations for nearly every known α6* nAChR population, it is demonstrated that α6* nAChRs on DA neurons are ˜10-fold more sensitive than any other relevant nAChR (FIG. 16). Classical intracranial injection experiments are certainly able to selectively stimulate groups of neurons, but are invasive and tedious; in particular, studies targeting DA nuclei such as the VTA or SNc cannot exclude the possibility of manipulating adjacent (for SNr) or co-distributed (for VTA) GABAergic neurons. Light-activated ion channels such as channelrhodopsin can be effectively stimulated with transcranial illumination when target structures are shallow, but implanted fiber optics are required to target deep structures such as VTA. α6^{L9′S} mice will be useful for studying the in vivo electrophysiological activity of any brain area of interest in response to specific DAergic stimulation. Further, α6^{L9′S} mice can also be used to study the acute, postsynaptic effects of striatal DA release.

Similar levels of αCtxMII binding were observed in α6^{L9′S} brains compared to WT controls (FIG. 9G). This is interesting in light of the fact that the two α6^{L9′S} lines harbor multiple copies of the transgene (FIG. 9D). Unlike an exon-replacement knock-in approach, α6^{L9′S} BAC transgenic mice retain two copies of the WT α6 locus. In DA neurons, α6 and α4 subunits compete for common, limiting nAChR subunits such as β2, possibly β3 and α5 (Gotti et al., 2008), and probably for unknown assembly factors or chaperone proteins that may be specific to this cell type. As a result, the level of functional α6* expression in α6^{L9′S} neurons is determined by a competition between WT and L9′S α6 subunits. Indeed, for every agonist tested, diminished peak αCtxMII-resistant (α4β2*-dependent) DA release was observed in α6^{L9′S} ST/OT (FIGS. 2 and 3) despite equal levels of α4β2* binding sites (FIG. 9G). Future crosses of α6^{L9′S} mice to α4, β2, and β3 nAChR heterozygous or homozygous KO mice will yield further insights into nAChR subunit stoichiometry in vivo.

A large increase in the potency and efficacy of nicotine in whole-cell recordings from α6^{L9′S} DA neurons (FIG. 4D) was noted. With respect to nicotinic channel engineering, the present studies are analogous to electrophysiological and Ca²⁺ flux-based measurements of hypersensitive nicotinic responses in α4^{L9′S}, α4^{L9′A}, and α7^{L9′T} knock-in mice (Fonck et al., J Neurosci 25:11396-11411, 2005; Labarca et al., Proc Natl Acad Sci USA 98:2786-2791, 2001; Orr-Urtreger et al., J Neurochem 74:2154-2166, 2000; Tapper et al., Science 306:1029-1032, 2004; Wooltorton et al., J Neurosci 23:3176-3185, 2003). The augmented responses (increased efficacy) to agonist likely reflect an increased maximal probability of channel opening, Pₒₚₑₙ, conferred by the L9′S mutation (Labarca et al., Nature 376:514-516, 1995), while the increased potency likely results from this mutation shifting the agonist concentrationresponse relation to lower concentrations. The increased noise in voltage clamp recordings from α6^{L9′S} neurons probably arises from one of two effects: 1) the presence of ACh in the slice preparation that is secreted from terminals of severed mesopontine cholinergic axons, or 2) unliganded openings.

α6^{L9′S} mice are viable and fertile, whereas full expression of α4^{L9′S}* receptors causes neonatal lethality, likely due to excitotoxic death of DA neurons (Labarca et al., Proc Natl Acad Sci USA 98:2786-2791, 2001). This could be due to differential expression of α4 and α6 in development; unlike α4 expression, peak α6 expression occurs well after birth. It is also possible that α6^{L9′S}* receptors are comparatively insensitive to activation by choline at concentrations found in CSF.

In the mesostriatal and mesolimbic DA system, α4β2* nAChRs are expressed in DA neuron cell bodies, dendrites and axon terminals, as well as in cell bodies and axon terminals of midbrain and striatal GABAergic neurons. Conclusive evidence for α6* nAChR expression, however, is restricted to DA neuron cell bodies and axon terminals. The present results show that, in midbrain, manipulating α6* nAChR sensitivity only affects DA neurons (FIG. 16, FIGS. 8B, and D), whereas sensitized α4* receptors simultaneously increase the sensitivity of DA neurons and their inhibitory GABAergic inputs (FIGS. 6, 8A, and 8C). This circuit-level difference explains a distinction between the locomotor effects of nicotine in the two gain-offunction mouse strains. WT mice display a hypolocomotor response to nicotine, and α4^{L9′A} mice recapitulate the WT response, only at much lower doses (Tapper et al., Physiol Genomics 31:422-428, 2007). On the other hand, α6^{L9′S} mice exhibit a sign change: psychomotor stimulation by nicotine (FIG. 1).

This cell-type difference in expression between α4 and α6 nAChR subunits may also lead to the behavioral differences between α4^{L9′A} and α6^{L9′S} mice in response to repeated nicotine injections. Repeated, selective activation of α4* nAChRs produces locomotor sensitization whereas repeated activation of α6* nAChRs produces neither tolerance nor sensitization (FIG. 1I). Locomotor sensitization may require nicotinic activation of GABAergic transmission, which is afforded by α4* nAChRs but not α6* nAChRs (Nashmi et al., J Neurosci 27:8202-18, 2007). Alternatively, the mechanism of sensitization could involve nAChR upregulation, to which α4β2* receptors are particularly prone, but to which α6* receptors are apparently resistant (Perry et al., J Pharmacol Exp Ther 322:306-315, 2007).

The VTA and NAc (mesolimbic DA pathway) are key mediators of the addictive properties of nicotine, and a recent report using pharmacological blockade suggests that α6* nAChRs specifically mediate cholinergic modulation of DA release in NAc (Exley et al., Neuropsychopharmacology 21:21, 2007). The results reported herein provide direct positive support for this in two ways: 1) greater α6*-dependent, nicotine-induced currents were observed in VTA versus SNc neurons (compare FIGS. 4E, 6D, and FIG. 13A), and 2) DA release is more strongly controlled by α6* from VTA-derived terminals versus SNc-derived terminals (comparing EC₅₀ values in FIGS. 2G and H). Although the contribution of dorsal versus ventral striatum in the behavioral experiments is not fully distinguished, the DA release data suggest that the first α6* nAChRs significantly activated by nicotine are those in the mesolimbic pathway.

VTA β2* nAChRs have been reported to be critical mediators of exploratory behavior in mice, The VTA is important for curiosity or the response to novelty, perhaps by responding to cholinergic excitation to mediate the switch to burst firing in DA neurons. If VTA β2* nAChRs are necessary for normal responses to novelty, then it is not surprising that sensitized VTA β2* nAChRs such as α6^{L9′S}β2* in mutant mice render the animals hypersensitive to novelty (FIGS. 1C and D). This phenotype is much smaller or absent in α4^{L9′A} mice, reflecting the relative importance of selectively activating DA in eliciting this response.

In midbrain, ACh released from mesopontine cholinergic terminals acts on DA neuron nAChRs in vivo (FIG. 8A). α6* nAChRs have the highest known sensitivity to ACh, making them excellent candidates to mediate the stimulatory action of endogenous ACh on DA neurons. The tonic activation of α6* nAChRs observed in midbrain slices is likely due to endogenous ACh. Although no difference was observed in DA neuron baseline firing in vitro (FIGS. 4I and 13B), tonic midbrain α6* nAChR activation may be sufficient in vivo to contribute to behavioral phenotypes in α6^{L9′S} mice. In striatum, tonic extracellular DA is controlled by presynaptic nAChRs via continuous, low-level ACh released from cholinergic interneurons. This acts to maintain a high probability of DA release from the terminal during tonic firing. Sensitization of α6* nAChRs (in α6^{L9′S} mice) likely modifies DA release at presynaptic terminals in addition to effects at DA neuron cell bodies; DA terminals with α6_{L9′S} channels presumably have a decreased failure rate for single spike-induced release. This is supported by in vitro electrochemical studies of α6*-dependent DA release. Thus, sensitization of DA neurons to ACh in midbrain and facilitation of DA release in striatum (FIG. 8B) could easily account for the home cage hyperactivity and the sustained hyperactivity of α6^{L9′S} mutant mice when placed in a novel environment. These phenotypes are reminiscent of DA transporter knock-down mice, which also show hyperactivity and impaired response habituation.

In α6^{L9′S} mice, low-dose nicotine stimulates psychomotor activation similar to amphetamine in WT animals (FIG. 1E). Nicotine thus recapitulates the spontaneous hyperactivity observed, though with different kinetics. The difference in response magnitude and duration between responses to novelty and responses to nicotine may reflect different agonist concentration and desensitization kinetics. After ACh is released from nerve terminals, it is hydrolyzed by acetylcholinesterase (AChE), which has a turnover rate of 10₄/s and is abundant in DAergic areas. ACh may not reach concentrations, or remain long enough, to significantly desensitize α6* nAChRs, perhaps even those with mutant L9′S subunits. In contrast, nicotine is eliminated with a half life of ˜7 min—nearly 10⁷ times longer—and can therefore desensitize receptors, especially β2* nAChRs. Bolus injections of nicotine in α6^{L9′S} mice potently activate mutant receptors, and the locomotor response decay kinetics could therefore be dominated by both receptor desensitization and metabolic breakdown of nicotine.

The cholinergic system is targeted by several drugs used to treat neural disorders such as Alzheimer's disease and Parkinson's disease (PD). There is a well-documented inverse correlation between smoking and PD, and other disorders that can be treated with DA drugs (ADHD, schizophrenia) are associated with a high incidence of smoking. These findings suggest the important role played by the cholinergic system in DA transmission. The results provided herein show that a sensitized response in DA neurons to endogenous ACh may cause a behaviorally relevant state of excess DA. Manipulations to decrease α6* nAChR function may, therefore, be a useful treatment for human disorders involving excess DA. This could be in the form of a competitive antagonist or via viral gene therapy designed to eliminate α6* activity. On the other hand, patients with PD (low DA) may be aided by α6* agonists or allosteric modulators to augment DA release from residual DA terminals. The data provided herein clearly show that an α6-selective compound potently stimulates both DA release and locomotor activity (FIG. 3). Further, the absence of sensitization or tolerance to repeated α6* activation (FIG. 1I) suggests that clinical α6* agonists may have reduced abuse liability. Unlike L-DOPA or direct DA receptor agonists/antagonists, compounds manipulating DA neuron firing by targeting α6* nAChRs might avoid well known use-dependent side effects such as dyskinesias.

As used herein, “non-transgenic mouse” refers to a wild-type mouse or a mouse in which the activity or expression of the nAChR subunit gene has not been manipulated. In such a non-transgenic mouse, the protein level and activity of the nAChR subunit would be expected to be within a normal range. As used herein, the term “wild type,” when used in reference to an animal, for example, a wild type mouse, refers to the animal as it exists in nature.

Nicotine-mediated behavior includes anxiety. A candidate agent having the ability to modulate nicotine-mediated behavior can decrease anxiety. Nicotine-mediated behavior also includes ambulation; a candidate agent having the ability to modulate nicotine-mediated behavior can decrease ambulation. Yet another nicotine-mediated behavior is motor learning; a candidate agent having the ability to modulate nicotine-mediated behavior can improve motor learning. Nicotine-mediated behaviors can be assessed by methods known to those of skill in the art and including described in Example 1.

In yet another embodiment of the present invention, there are provided methods for screening a candidate agent for the ability to modulate nicotine-mediated behavior in the transgenic animal having a transgene encoding a variant nicotinic acetylcholine receptor (nAChR) subunit, wherein the variant nAChR subunit is selected from the group consisting of α6, α5, and β2, and wherein further the expression of the variant results in an animal that displays a modified phenotype compared to a wild type animal. The method includes administering to a first transgenic animal a candidate agent, and comparing nicotine-mediated behavior of the first transgenic animal to nicotine-mediated behavior of a second transgenic animal not administered the candidate agent. A difference in nicotine-mediated behavior in the first transgenic animal administered the candidate agent compared to the second transgenic animal not administered the candidate agent is indicative of a candidate agent that modifies nicotine-mediated behavior. In some embodiments, the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.

In yet another embodiment of the present invention, there are provided methods for screening for candidate agent that modulates a nicotinic acetylcholine receptor (nAChR) subunit, in which the method includes administering a candidate agent to a transgenic animal of the invention and determining the effect of the agent upon a cellular or molecular process associated with nicotinic hypersensitivity compared to an effect of the agent administered to a non-transgenic animal. In such methods, a difference in effect is indicative of an agent that modulates nicotine hypersensitivity.

In still another embodiment of the present invention, there are provided methods for screening for candidate agent that modulates nicotine hypersensitivity, in which the method includes administering a candidate agent to a transgenic animal containing a variant nicotinic acetylcholine receptor (nAChR) subunit and comparing nicotine-mediated behavior of the first transgenic animal to nicotine-mediated behavior of a non-transgenic littermate animal administered the same dose of the candidate agent, wherein a difference in effect is indicative of an agent that modulates the nicotinic acetylcholine receptor (nAChR) subunit. In some embodiments, the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.

The term “candidate agent” is used herein to mean any agent that is being examined for ability to modulate nicotine-mediated activity in a method of the invention. Although the method generally is used as a screening assay to identify previously unknown molecules that can act as a therapeutic agent, a method of the invention also can be used to confirm that an agent known to have such activity, in fact has the activity, for example, in standardizing the activity of the therapeutic agent.

A candidate agent can be any type of molecule, including, for example, a peptide, a peptidomimetic, a polynucleotide, or a small organic molecule, that one wishes to examine for the ability to act as a therapeutic agent, which is an agent that provides a therapeutic advantage to a subject receiving it. It will be recognized that a method of the invention is readily adaptable to a high throughput format and, therefore, the method is convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. Nos. 5,622,699; 5,206,347; Scott and Smith, Science 249:386-390, 1992; Markland et al., Gene 109:1319, 1991; each of which is incorporated herein by reference); a peptide library (U.S. Pat. No. 5,264,563, which is incorporated herein by reference); a peptidomimetic library (Blondelle et al., Trends Anal. Chem. 14:8392, 1995; a nucleic acid library (O'Connell et al., supra, 1996; Tuerk and Gold, supra, 1990; Gold et al., slpra, 1995; each of which is incorporated herein by reference); an oligosaccharide library (York et al., Carb. Res., 285:99128, 1996; Liang et al., Science, 274:1520-1522, 1996; Ding et al., Adv. Expt. Med. Biol., 376:261-269, 1995; each of which is incorporated herein by reference); a lipoprotein library (de Kruif et al., FEBS Lett., 399:232-236, 1996, which is incorporated herein by reference); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference). Accordingly, the present invention also provides a therapeutic agent identified by such a method, for example, a neuroactive therapeutic agent.

The route of administration of a candidate agent will depend, in part, on the chemical structure of the candidate agent. Peptides and polynucleotides, for example, are not particularly useful when administered orally because they can be degraded in the digestive tract. However, methods for chemically modifying peptides, for example, to render them less susceptible to degradation by endogenous proteases or more absorbable through the alimentary tract are well known (see, for example, Blondelle et al., Trends Anal. Chem. 14:83-92, 1995; Ecker and Crooke, Bio/Technology, 13:351-360, 1995; each of which is incorporated herein by reference). In addition, a peptide agent can be prepared using D-amino acids, or can contain one or more domains based on peptidomimetics, which are organic molecules that mimic the structure of peptide domain; or based on a peptoid such as a vinylogous peptoid.

A candidate agent can be administered to an individual by various routes including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the candidate agent can be administered by injection, intubation, orally or topically, the latter of which can be passive, for example, by direct application of an ointment, or active, for example, using a nasal spray or inhalant, in which case one component of the composition is an appropriate propellant.

The total amount of a candidate agent to be administered in practicing a method of the invention can be administered to a subject as a single dose, either as a bolus or by infusion over a relatively short period of time, or can be administered using a fractionated treatment protocol, in which multiple doses are administered over a prolonged period of time. The candidate agent can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

In another embodiment of the present invention, there are provided methods for screening a candidate agent for the ability to modulate nicotine-mediated behavior in the transgenic animal of the invention. The method includes administering to a first transgenic animal a candidate agent, and comparing nicotine-mediated behavior of the first transgenic animal to nicotine-mediated behavior of a second transgenic animal not administered the candidate agent. A difference in nicotine-mediated behavior in the first transgenic animal administered the candidate agent compared to the second transgenic animal not administered the candidate agent is indicative of a candidate agent that modifies nicotine-mediated behavior.

Also provided by the invention is a method of screening for biologically active agents that modulate nicotine hypersensitivity. The method comprises administering a candidate agent to a transgenic animal and determining the effect of the agent upon a phenomenon associated with nicotinic hypersensitivity compared to an effect of the agent administered to a nontransgenic animal. A phenomenon associated with nicotinic hypersensitivity is dopaminergic neuronal cell loss. Methods to assess loss of dopaminergic neuronal cells are known in the art, and include immunohistochemical and anatomical methods, and methods described in for example U.S. Pat. No. 6,753,456.

The invention will now be described in greater detail by reference to the following non-limiting examples.

### EXAMPLE 1

In the present example, mice with gain-of-function α6* nAChRs, which isolate and amplify cholinergic control of DA neuron activity, were generated. In contrast to gene knockouts or pharmacological blockers, which show necessity, activating α6* nAChRs and DA neurons was shown to be sufficient to cause locomotor hyperactivity. α6^{L9′S} mice were hyperactive in their home cage and failed to habituate to a novel environment. Specific activation of α6* nAChRs with low doses of nicotine, by stimulating DA but not GABA neurons, recapitulated these spontaneous phenotypes and produced a hyperdopaminergic state in vivo. Experiments with additional nicotinic drugs showed that altering agonist efficacy at α6* provides fine-tuning of DA release and locomotor responses. α6*-specific agonists or antagonists may, by targeting endogenous cholinergic mechanisms, provide a new method for manipulating DA transmission in Parkinson's disease, nicotine dependence, or attention deficit hyperactivity disorder (ADHD).

Materials. [³H]-dopamine was obtained from Perkin Elmer (Boston, Mass.) (7,8-[³H] at 30-50 Ci/mmol). Hepes, half sodium salt, was a product of Roche Applied Science (Indianapolis, Ind.). Ultra centrifugation grade sucrose was obtained from Fisher Chemicals (Fairlawn, N.J.). Sigma-Aldrich (St. Louis, Mo.) was the source for the following compounds: L-(+)-arabinose, ascorbic acid, atropine sulfate, bovine serum albumin (BSA), (−)-nicotine tartrate, nomifensine, mecamylamine, R(+)-SCH23390, streptomycin, ampicillin, chloramphenicol, kanamycin, tetracycline, yohimbine, prazosin, and pargyline. Optiphase ‘SuperMix’ scintillation fluid was from Perkin Elmer Life Sciences-Wallac Oy, (Turku, Finland).

Mice. All experiments were conducted in accordance with the guidelines for care and use of animals provided by the National Institutes of Health, and protocols were approved by the Institutional Animal Care and Use Committee at the California Institute of Technology, the University of Colorado Boulder, or the Rockefeller University. Mice were kept on a standard 12 h light/dark cycle at 22° C. and given food and water ad libitum. On postnatal day 21, mice were weaned and housed with same-sex littermates. At 21 to 28 days, tail biopsies were taken for genotype analysis by PCR. Tail biopsies were digested in 50 mM NaOH at 95° C. for 45 minutes followed by neutralization with 0.5 M Tris-Cl, pH 5.5 and subsequent direct analysis by multiplex PCR. α4^{L9′A} knock-in mice used in this study were generated on a mixed C57/129Sv background (Fonck et al., J Neurosci 25(49):11396-411, 2005; Tapper et al., Science 306:1029-32, 2004; and U.S. Pat. No. 6,753,456), and were backcrossed at least 10 generations to C57BL/6. Wild type (WT) control mice were littermates of α6^{L9′S} transgenic mice.

Bacterial Artificial Chromosome Recombineering and Transgenesis. A bacterial artificial chromosome (BAC) RP24-149112 containing the mouse α6 nicotinic receptor subunit gene (Chrna6) was obtained from the BACPAC Resource Center (BPRC) at Children's Hospital Oakland Research Institute (Oakland, Calif.). BAC strains were maintained according to standard molecular biology and sterile techniques. BAC recombineering was carried out using a Counter Selection BAC modification kit (Genebridges; Heidelberg, Germany). Recombineering in bacteria utilizes endogenous recombination activity, and allows the insertion of exogenous DNA into the BAC without residual sequences such as selection markers (neo) or loxP sites. α6 Leucine 280 (Leu9′) was mutated to a serine using a two-step selection/counter selection protocol in E. coli. First, a 15 bp stretch of α6 exon 5 (5′-gttctgcmctctc-3′; SEQ ID NO:1) containing the coding sequence for V278 through L282 (which includes the Leu9′ residue) was replaced with a cassette containing a tandem selection (neo)/counter selection (rpsL) marker. The rpsL/neo cassette was amplified by PCR using oligos designed to engineer α6 exon 5 homology arms flanking the sequence between and including V278 to L282. The oligo sequences were: forward primer: 5′-tt ttt tac ctt ccc tcc gac tgt ggc gag aaa gtg act ctt tgc atc tcc gcggccgc GGC CTG GTG ATG ATG GCG GGA TCG-3′, SEQ ID NO:2; and reverse primer: 5′-ac gag aga tgt gga tgg gat ggt ctc tgt aat cac cag caa aaa gac agt gcggccgc TCA GAA GAA CTC GTC AAG AAG GCG-3′, SEQ ID NO:3 (homology arms: lower case; NotI restriction site: underlined, lower case; rpsL/neo cassette priming sequence: upper case).

Neo was used to select positive recombinants, and an engineered NotI restriction site pair flanking the selection cassette was used to confirm the location of the exogenously inserted DNA within the BAC. In the second step, α6 exon 5 was restored using counter selection. Bacterial cells were placed under selective pressure (via streptomycin sensitivity gained by insertion of the rpsL marker) to lose the neo-rpsL cassette and replace it with non-selectable DNA engineered to insert the Leu9′ to Ser (L280S) mutation. Non-selectable DNA sense strand sequence was: 5′-tt ttt tac ctt ccc tcc gac tgt ggc gag aaa gtg act ctt tgc atc tcc gtt ctg TCA agc ttg act gtc ttt ttg ctg gtg att aca gag acc atc cca tcc aca tct ctc gt-3′, SEQ ID NO:4 (L280S mutation in upper case, silent HindIII restriction site underlined). The resultant strain harbored a BAC with no ectopic DNA in or around the α6 gene. α6^{L9′S} BAC DNA was confirmed to have the desired mutation by DNA sequencing, restriction mapping, and diagnostic PCR. The α6 nicotinic receptor gene is directly adjacent to the β3 nicotinic receptor gene (Chrnb3). To eliminate the possibility that any physiological or behavioral phenotypes of our transgenic mice could be attributed to the presence of extra copies of β3, but to retain the β3 locus, the β3 gene was silenced using homologous recombination. β3 was silenced by replacing exon 1 (containing the methionine initiation codon) with an ampicillin selection cassette. An ampicillin marker derived from pcDNA3.1zeo was amplified by PCR using oligos designed to engineer β3 homology arms and diagnostic SbfI restriction sites flanking the ampicillin marker. The oligo sequences were: forward primer: 5′-agc ctc aca aga cct gac agc tca ctg ggc atc agt gaa gtg cac cctgcagg GAC GTC AGG TGG CAC-3′, SEQ ID NO:5 and reverse primer: 5′-tga gag agt ggc act gag agc caa gaa gac ccg tag gaa gcc tgt cctgcagg GTC TGA CGC TCA GTG-3′, SEQ ID NO:6 (homology arms: lower case; SbfI restriction site: underlined, lower case; ampicillin marker priming sequence: upper case). Two additional genes (4921537β18Rik and Tex24) which are not expressed in brain were also contained on the final BAC construct.

Injection-grade α6^{L9′S} BAC DNA was prepared via double CsCl banding (Lofstrand Labs; Gaithersberg, Md.). To produce transgenic animals, BAC DNA was injected into the male pronucleus of recently fertilized FVB/N embryos and implanted into pseudopregnant Swiss-Webster surrogates. Transgenic founders were identified using tail biopsy DNA and PCR primers designed to detect both the L9′S mutation (forward: 5′-ctc cgt tct gtc aag ctt g-3′, SEQ ID NO:7; reverse: 5′-acg agt gct ctg aat tct ctg-3′, SEQ ID NO:8), and the inserted ampicillin cassette within the 13 gene (forward: 5′-gct cat gag aca ata acc ctg-3′, SEQ ID NO:9; reverse: 5′-cag tct tgg aag caa cat cca gc-3′, SEQ ID NO:10). Founders were crossed to C57BL/6J (Jackson Labs; Bar Harbor, Me.) to obtain germline transmission and to establish a colony, and transgenic mice were continually backcrossed to C57BL/6J. Routine genotyping was done by multiplex PCR (forward primer #1: 5′-ctc cgt tct gtc aag ctt g-3′, SEQ ID NO:7; forward primer #2: 5′-ctg ctg ctc atc acc gag atc-3′, SEQ ID NO:11; reverse primer #1: 5′-acg agt gct ctg aat tct ctg-3′, SEQ ID NO:8; reverse primer #2: 5′-cag atg tca ccc aag atg ccg-3′, SEQ ID NO:12) analysis of tail DNA from newly weaned mice.

Real Time PCR. Mouse genomic DNA was obtained from tail biopsies. Relative quantification of total α6 gene copies was performed using the LightCycler 480 system and SYBR Green I Master Mix (Roche Diagnostics; Indianapolis, Ind.). The α6 (WT and L9′S alleles) locus was detected with an α6-specific primer set (forward primer: 5′-gag cgc tgc tga cac ttg-3′, SEQ ID NO:13; reverse primer: 5′-ccc ctt gta gca cct agc-3′, SEQ ID NO:14). The α4 nicotinic receptor genomic locus, which is assumed to be present at one copy per haploid genome, was used as a reference target and was detected with α4-specific primers (forward primer: 5′-ctg ctg ctc atc acc gag atc-3′, SEQ ID NO:11; reverse primer: 5′-cag atg tca ccc aag atg ccg-3′, SEQ ID NO:15). Crossing point values were obtained for target (α6) and reference (α4) genes in serial dilutions of gDNA from non-transgenic and transgenic mice. The relative ratio of α6 genomic copies in non-transgenic versus transgenic mice was calculated according to accepted standards.

RT-PCR. For RNA analysis, mice were anesthetized with halothane and sacrificed by cervical dislocation. Brains were rapidly removed and extracted in ice-cold Trizol (Invitrogen; Carlsbad, Calif.) (1 ml Trizol per 100 mg wet brain tissue) aided by dounce homogenization. RNA was purified according to the manufacturer's instructions, resuspended in DEPC-treated water, and stored at −80° C. RNA quality was assessed by observing absorbance profiles across a range of wavelengths between 220 nm and 320 nm. Spectrophotometric analysis was performed using a ND-1000 spectrophotometer (NanoDrop; Wilmington, Del.). Reverse transcription and target amplification was performed using SUPERSCRIPT III One-Step RT-PCR enzyme mix (Invitrogen). Gene-specific primers for total α6 mRNA (mutant and WT; forward primer: 5′-gtt tta cac cat caa cct cat c-3′, SEQ ID NO:16; reverse primer: 5′-tta gga gtc tgt gta ctt ggc-3′, SEQ ID NO: 17) or α6_{L9′S} mRNA (forward primer: 5′-ctc cgt tct gtc aag ctt g-3′, SEQ ID NO:7; reverse primer: 5′-acg agt gct ctg aat tct ctg-3′, SEQ ID NO:8) were used to prime first-strand cDNA synthesis and subsequent double-stranded PCR product. In control reactions, Taq DNA polymerase was substituted for SuperScript III enzyme mix.

⁸⁶Rb⁺Efflux from Superior Colliculus Synaptosomes. Nicotine-stimulated ⁸⁶Rb⁺ efflux from superior colliculus (SC) was measured as follows. SC was dissected from adult mice sedated with halothane and sacrificed by cervical dislocation. Tissue was homogenized by hand in 1 ml of ice-cold 0.32 M sucrose buffered to pH 7.5 with HEPES using a glass/Teflon tissue grinder. After homogenization, the grinder was rinsed three times with 0.5 ml of buffered sucrose solution. A crude synaptosomal pellet was obtained by centrifugation at 12,000 g for 20 min. After removal of the sucrose, each pellet was resuspended in load buffer (140 mM NaCl, 1.5 mM KCl, 2 mM CaCl₂, 1 mM MgSO₄, 25 mM HEPES, and 22 mM glucose) and placed on ice until incubation with ⁸⁶RbCl. SC synaptosomes were incubated with 4 μCi of ⁸⁶Rb⁺ for 30 min in a final volume of 35 μl of load buffer, after which samples were collected by gentle filtration onto a 6-mm-diameter Gelmantype A/E glass filter and washed once with 0.5 ml of load buffer. Filters containing the synaptosomes loaded with ⁸⁶Rb±were transferred to a polypropylene platform and superfused for 5 min with effluent buffer (135 mM NaCl, 1.5 mM KCl, 5 mM CsCl, 2 mM CaCl₂, 1 mM MgSO₄, 25 mM HEPES, 22 mM glucose, 50 nM tetrodotoxin, and 0.1% bovine serum albumin). A peristaltic pump applied buffer to the top of the synaptosome containing filter at a rate of 2 ml/min, and a second peristaltic pump set at a faster flow rate of 3 ml/min removed buffer from the bottom of the platform. The greater speed of the second pump prevented pooling of buffer on the filter. Effluent buffer was pumped through a 200 μl Cherenkov cell and into a P-Ram detector (IN/US Systems; Tampa, Fla.). Radioactivity was measured for 3 min with a 3 s detection window providing 60 data points for each superfusion. Each aliquot was stimulated by 1 of 4 different nicotine concentrations, with a 5 s exposure for each concentration.

[¹²⁵I]-α-conotoxin MII Binding to Brain Sections. Two mice for each genotype (WT, α6^{L9′S} line 2 and line 5) were sedated with halothane and sacrificed by cervical dislocation. The brains were removed and rapidly frozen by immersion in isopentane (−35° C., 60 s). The frozen brains were wrapped in aluminum foil, packed in ice, and stored at −70° C. until sectioning. Tissue sections (14 μm) prepared using an IEC Minotome Cryostat refrigerated to −16° C. were thaw mounted onto Fisher Superfrost/Plus Microscope Slides. Mounted sections were stored desiccated at −70° C. until use. Eight series of sections were collected from each mouse brain.

Before incubation with [¹²⁵I]-αCtxMII, three adjacent series of sections from each mouse were incubated in binding buffer (144 mM NaCl, 1.5 mM KCl, 2 mM CaCl₂, 1 mM MgSO₄, 20 mM HEPES, 0.1% BSA (w/v), pH 7.5) containing 1 mM phenylmethylsulfonyl fluoride at 22° C. for 15 min. For all [¹²⁵I]-αCtxMII binding reactions, the standard binding buffer was supplemented with BSA [0.1% (w/v)], 5 mM EDTA, 5 mM EGTA, and 10 mg/ml each of aprotinin, leupeptin trifluoroacetate, and pepstatin A to protect the ligand from endogenous proteases. The sections were then incubated with 0.5 nM [¹²⁵I]-αCtXMII for 2 h at 22° C. Samples were washed as follows: once for 30 sec in binding buffer plus 0.1% BSA at 22° C., twice for 30 sec in binding buffer plus 0.1% BSA (4° C.), twice for 5 sec in 0.1× binding buffer plus 0.01% BSA (4° C.) and twice for 5 sec in 5 mM HEPES, pH 7.5 (0° C.). Sections were initially dried with a stream of air, then by overnight storage (22° C.) under vacuum. Mounted, desiccated sections were apposed to film (1-3 days, Kodak MR film).

[¹²⁵I]-Epibatidine Binding to Membranes. Each mouse was sacrificed by cervical dislocation. The brain was removed and placed on an ice-cold platform. Tissue was collected from superior colliculus, thalamus, striatum (CPu and dorsal NAc), and olfactory tubercle, then homogenized in ice-cold hypotonic buffer (144 mM NaCl; 0.2 mM KCl; 0.2 mM CaCk; 0.1 mM MgSO₄; 2 mM HEPES; pH 7.5) using a glass-Teflon tissue grinder. Particulate fractions were obtained by centrifugation at 20,000 g (15 min, 4° C.; Sorval RC-2B centrifuge). The pellets were resuspended in fresh homogenization buffer, incubated at 22° C. for 10 min, then harvested by centrifugation as before. Each pellet was washed twice more by resuspension/centrifugation, then stored (in pellet form under homogenization buffer) at −70° C. until use. Protein was quantified with a Lowry assay using bovine serum albumin as the standard.

Binding of [¹²⁵I]-epibatidine was quantified using a modified version of the methods previously described for [³H]-epibatidine. Incubations were performed in 96-well polystyrene plates, in 30 ml of binding buffer (144 mM NaCl; 1.5 mM KCl; 2 mM CaCl₂; 1 mM MgSO₄; 20 mM HEPES; pH 7.5). Plates were covered to minimize evaporation during incubation, and all incubations progressed for 2 h at 22° C. Saturation binding experiments were performed for membrane preparations from each brain region, using a [¹²⁵I]-epibatidine ligand concentration of 200 pM. Binding reactions were terminated by filtration of samples onto a single thickness of polyethyleneimine-soaked (0.5% w/v in binding buffer) GFA/E glass fiber filters (Gelman Sciences; Ann Arbor, Mich.) using an Inotech Cell Harvester (Inotech; Rockville, Md., U.S.A.). Samples were subsequently washed six times with ice-cold binding buffer. Bound ligand was quantified by gamma counting at 83-85% efficiency, using a Packard Cobra counter. In experiments with competitive, unlabeled αCtxMII, the amount of membrane protein added was chosen to produce maximum binding of ligand to the tissue of approximately 40 Bq/well (less than 10% of total ligand added, minimizing the effects of ligand depletion). For αCtxMII (50 nM), the medium was supplemented with bovine serum albumin (0.1% w/v) as a carrier protein. For all experiments, non-specific binding was determined in the presence of 1 mM (−)-nicotine tartrate.

Immunohistochemistry and Spectral Confocal Imaging Coronal brain slices cut for patch-clamp recording were removed from the recording chamber and immediately fixed (4% PFA in PBS, pH 7.4) for 45 min at 4° C. Slices were permneabilized (20 mM Hepes, pH 7.4, 0.5% Triton X-100, 50 mM NaCl, 3 mM MgCl₂, 300 mM sucrose) for 1 h at 4° C. followed by blocking (0.1% Triton X-100, 5% donkey serum in TBS) for 1 h at room temperature. Slices were incubated overnight at 4° C. in rabbit anti-tyrosine hydroxylase (TH) primary antibody (Pel-Freez; Rogers, Ark.) (diluted 1:100 in 0.1% Triton X-100, 5% donkey serum in TBS), washed three times in TBST (0.1% Triton X-100 in TBS), incubated for 1 h at room temperature in goat anti-rabbit Alexa 488 secondary antibody (Molecular Probes; Eugene, Oreg.) (diluted 1:500 in 0.1% Triton X-100, 5% donkey serum in TBS), and washed three times in TBST.

Slices were mounted and imaged with a Nikon (Nikon Instruments, Melville, N.Y.) C1 laser-scanning confocal microscope system equipped with spectral imaging capabilities and a Prior (Rockland, Me.) remote-focus device. A Nikon Plan Apo 10× objective was used, and pinhole diameter was 30 μm. Sections were imaged at 12-bit intensity resolution over 512×512 pixels at a pixel dwell time of 6 μs. Alexa 488 was excited with an argon laser at 488 nm. Imaging was carried out using the Nikon C1si DEES grating and spectral detector with 32 parallel photomultiplier tubes. Signal from Alexa 488 was unmixed from background autofluorescence similar to other studies.

Statistics and Data Analysis. Physiology, neurochemistry, and real time PCR data were reported as mean±SEM. Statistical significance (p<0.05) was determined with at test for continuous data meeting parametric assumptions for normality and equal variance. DA release parameters and behavior data were analyzed for significance with two-way or one-way ANOVA, respectively, with Tukey post hoc analysis.

Locomotor Activity. Mice used to study locomotion were eight to sixteen weeks old by the beginning of an experiment, and were back-crossed to C57BL/6 at least three times. Back-crossing to C57BL/6 did not affect locomotor activity. Mouse horizontal locomotor activity was measured with an infrared photobeam activity cage system (San Diego Instruments; San Diego, Calif.). Ambulation events were recorded when two contiguous photobeams were broken in succession. Acute locomotor activity in response to nicotinic ligands or other agents was studied by recording ambulation events during four 15 sec intervals per minute for a designated number of minutes. For most experiments, groups of eight mice were placed in the activity cages (18×28 cm) and their baseline level of activity was recorded for eight minutes. Mice were removed from their cage, injected (100 μl per 25 g body mass), and returned to the cage within 15 sec. For some experiments, mice were pre-injected with saline or an antagonist immediately prior to the start of the experiment, followed by challenge with nicotine eight minutes after the start of the experiment. For generation of locomotor activity concentration-response relationship profiles, a group of mice was administered saline and, after five to eight days off, each successive dose of drug. For experiments probing sensitization or tolerance, mice were injected once daily with saline for three days prior to the start of daily nicotine injections. For 48 h home cage monitoring, mice were isolated in their own cage and habituated to the test room and cage for 24 h. Following this, locomotor activity was recorded in 15 min intervals for 48 h.

Neurotransmitter Release from Striatal Synaptosomes. After a mouse was sacrificed by cervical dislocation, its brain was removed and placed immediately on an ice-cold platform and brain regions were dissected. Tissues from each mouse were homogenized in 0.5 ml of ice-cold 0.32 M sucrose buffered with 5 mM HEPES, pH 7.5. A crude synaptosomal pellet was prepared by centrifugation at 12,000 g for 20 min. The pellets were resuspended in “uptake buffer”: 128 mM NaCl, 2.4 mM KCl, 3.2 mM CaCl₂, 1.2 mM KH₂PO₄, 1.2 mM MgSO₄, 25 mM HEPES, pH 7.5, 10 mM glucose. For DA uptake, buffer was supplemented with 1 mM ascorbic acid and 0.01 mM pargyline, whereas for GABA uptake, buffer was supplemented with 1 mM aminooxyacetic acid. For [³H]GABA uptake, synaptosomes were incubated for 10 min at 37° C. [³H]GABA and unlabeled GABA were then added to final concentrations of 0.1 and 0.25 μM, respectively, and the suspension was incubated for another 10 min. For DA uptake, synaptosomes were incubated at 37° C. in uptake buffer for 10 min before addition of 100 nM [³H]dopamine (1 μCi for every 0.2 ml of synaptosomes), and the suspension was incubated for an additional 5 min.

All experiments were conducted at room temperature using methods described previously (Nashmi et al., 2007; Salminen et al., 2007) with modifications for collection into 96-well plates. In brief, aliquots of synaptosomes (80 μl) were distributed onto filters and perfused with buffer (uptake buffer containing 0.1% bovine serum albumin and 1 μM atropine with 1 μM nomifensine (for DA release) or 0.1 μM NNC-711 [1-(2-(((diphenylmethylene)amino)oxy)ethyl)-1,2,5,6-tetrahydro-3-pyridinecarboxylic acid hydrochloride]) (for GABA release) at 0.7 ml/min for 10 min, or buffer for 5 min followed by buffer with 50 nM αCtxMII. Aliquots of synaptosomes were then exposed to nicotine or high potassium (20 mM) in buffer for 20 sec to stimulate release of [³H]dopamine or [³H]GABA, followed by buffer. Fractions (−0.1 ml) were collected for 4 min into 96-well plates every 10 sec starting from 1 min before stimulation, using a Gilson FC204 fraction collector with a multicolumn adapter (Gilson, Inc.; Middleton, Wis.). Radioactivity was determined by scintillation counting using a 1450 MicroBeta Trilux scintillation counter (Perkin Elmer Life Sciences-Wallac Oy; Turku, Finland) after addition of 0.15 ml Optiphase ‘SuperMix’ scintillation cocktail. Instrument efficiency was 40%.

Data were analyzed using SigmaPlot 5.0 for DOS. Perfusion data were plotted as counts per minute versus fraction number. Fractions collected before and after the peak were used to calculate baseline as a single exponential decay. The calculated baseline was subtracted from the experimental data. Fractions that exceeded baseline by 10% or more were sunmmed to give total released cpm and then normalized to baseline to give units of release [(cpm-baseline)/baseline]. Agonist dose response data were fit to the Hill equation.

Patch-Clamp Electrophysiology. For slice electrophysiology, transgenic and non-transgenic mice were identified by genotyping new litters at approximately 14 days after birth. Postnatal day 17-25 (for midbrain), 21-28 (for locus coeruleus), or 42-56 (for striatum) mice were anesthetized with sodium pentobarbital (40 mg/kg, i.p.) followed by cardiac perfusion with oxygenated (95% O₂/5% CO₂) ice-cold glycerol-based artificial CSF (gACSF) containing 252 mM glycerol, 1.6 mM KCl, 1.2 mM NaH2PO₄, 1.2 mM MgCl₂, 2.4 mM CaCl₂, 18 mM NaHCO₃, and 11 mM glucose. Following perfusion, brains were removed and retained in gACSF (0-4° C.). Coronal slices (midbrain, striatum: 250 μm; pons: 200 μm) were cut with a microslicer (DTK-1000; Microslicer; Ted Pella, Redding, Calif.) at a frequency setting of 9 and a speed setting of 3.25. Brain slices were allowed to recover for at least 1 h at 32° C. in regular, oxygenated artificial CSF (ACSF) containing 126 mM NaCl, 1.6 mM KCl, 1.2 mM NaH2PO₄, 1.2 mM MgCl₂, 2.4 mM CaCl₂, 18 mM NaHCO₃, and 11 mM glucose.

For recordings, a single slice was transferred to a 0.8 ml recording chamber (Warner Instruments, RC-27 L bath with PH-6D heated platform). Slices were continually superfused with ACSF (1.5-2.0 ml/min) throughout the experiment. Cells were visualized with an upright microscope (BX 50WI; Olympus) and near-infrared illumination. Patch electrodes were constructed from Kwik-Fil borosilicate glass capillary tubes (1B150F-4; World Precision Instruments, Inc.; Sarasota, Fla.) using a programmable microelectrode puller (P-87; Sutter Instrument Co.; Novato, Calif.). The electrodes had tip resistances of 4.5-8.0 MΩ when filled with internal pipette solution (pH adjusted to 7.25 with Tris base, osmolarity adjusted to 290 mOsm with sucrose) containing: 135 mM potassium gluconate, 5 mM EGTA, 0.5 mM CaCk2, 2 mM MgCh, 10 mM HEPES, 2 mM Mg-ATP, and 0.1 mM GTP.

Whole-cell recordings were taken at 32° C. with an Axopatch 1D amplifier, a 16-bit Digidata 1322A A/D converter, and pCLAMβ9.2 software (all Molecular Devices Axon; Sunnyvale, Calif.). Data were sampled at 5 kHz and low-pass filtered at 1 kHz. The junction potential between the patch pipette and the bath solution was nulled immediately prior to gigaseal formation. Series resistance was uncompensated. Putative GABAergic neurons in the SNr and DAergic neurons in SNc or VTA were identified according to generally accepted criteria (Nashmi et al., J Neurosci 27, 8202-8218, 2007; Wooltorton et al., J Neurosci 23, 3176-85, 2003): (1) narrow spikes in GABA neurons vs. broad spikes in DA neurons; (2) rapid firing (>10 Hz) in GABA neurons vs. slow firing (<5 Hz) in DA neurons; (3) SNc DA neurons and to a lesser degree VTA DA neurons, but not SNr GABAergic neurons, express hyperpolarization-activated cation current (Iₕ). Noradrenergic neurons in locus coeruleus were identified by the following criteria: (1) expression of tyrosine hydroxylase as determined by immunohistochemistry; (2) pacemaker firing (<2 Hz); (3) absence of Iₕ currents or significant adaptation of membrane potential in response to hyperpolarizing current injection; (4) location immediately medial to medial parabrachial nucleus. Striatal cholinergic interneurons were identified by the following criteria: 1) cholinergic cells are rare relative to medium spiny neurons, large (>20 μm), and have 1-3 primary dendrites; 2) expression of Iₕ currents and significant membrane potential sag in response to hyperpolarizing current injection; 3) some cells do not fire spontaneously whereas other exhibit tonic, irregular firing (0-5 Hz).

To examine the function of somatic nAChRs, nicotine was locally applied using a Picospritzer II (General Valve; Fairfield, N.J.). Using a piezoelectric translator (Burleigh Instruments; Fishers Park, N.Y.), the pipette tip was moved within 20-40 μm of the recorded cell over a period of 250 ms starting 300 ms before drug application. Nicotine was then puffed at 10-20 psi for 250 ms. Fifty milliseconds after the application, the glass pipette was retracted over a period of 250 ms.

Production and Characterization of BAC α6_{L9′S} Transgenic Mice. A 156 kb mouse BAC clone containing the genomic Chrna6 (α6 nAChR) locus with substantial 5′ and 3′ flanking genomic regions was selected for generating a targeting construct to faithfully recapitulate expression of the α6 gene. α6 Leu280 (the Leu 9′ residue in the M2 domain) was mutated to Ser via homologous recombination using a two-step selection/counter selection procedure in E. coli. (FIG. 9A). The final construct was injected into fertilized mouse eggs and six transgenic offspring were identified by genomic DNA sequencing and diagnostic PCR (FIGS. 9B and C). FIG. 1B shows the genomic DNA sequence of WT and transgenic mice and demonstrates the presence of WT and Leu9′Ser α6 alleles in transgenic mice, and only WT α6 alleles in non-transgenic mice. To eliminate possible artifacts of transgene position/insertion, two independently derived lines (lines ‘2’ and ‘5’) were analyzed, which have different transgene copy numbers (FIG. 9D) and different genomic positions. Both mouse lines expressed mutant α6^{L9′S} mRNA in addition to wild type (WT) α6 mRNA (FIG. 9E).

No difference was found in location or intensity of [¹²⁵I]-α-conotoxin MII (αCtxMII) labeling in α6^{L9′S} versus WT brains, confirming correct regional expression of α6* nAChRs in mutant mice (FIG. 9F). To corroborate this, α6* binding sites were quantified. Membranes were prepared from striatum (ST), olfactory tubercle (OT), superior colliculus (SC) (regions which account for most α6* binding sites), and thalamus (TH) (where most binding sites are α4β2*). [¹²⁵]I-epibatidine binding, coupled with inhibition by unlabeled αCtxMII, revealed α6* and non-α6* (α4β2*) receptors (FIG. 9G). Collectively, these results indicate that α6^{L9′S} mice exhibit normal levels and localization of neuronal nAChRs.

Spontaneous and Nicotine-Induced Hyperactivity in α6_{L9′S} Mice. Home cage horizontal locomotor activity was measured for WT and α6^{L9′S} mice over a period of 48 h. α6_{L9′S} mice were markedly hyperactive relative to WT control littermates (FIG. 1A). This effect was restricted to lights off (FIG. 1B), though there was a nonsignificant trend toward hyperactivity during lights on. Although WT mice show locomotor habituation when placed into a novel environment, α6^{L9′S} mice exhibit sustained activity. We measured WT and α6^{L9′S} locomotor activity for 30 min after placement in a new home cage environment (FIG. 1C). Activity during the first 15 min of the session was unchanged, but from t=16 to 30 min, α6_{L9′S} mice exhibited significantly greater activity than WT littermates (FIG. 1D).

α6* nAChRs are highly expressed in DAergic neurons, and nicotine has psychomotor stimulant properties in rodents. WT and α6^{L9′S} mice were injected with nicotine and measured locomotor activity. Low doses of nicotine (0.15 mg/kg, i.p.) strongly activated locomotion in α6^{L9′S} mice but had no effect in WT mice (FIG. 1E). To characterize the locomotor activation phenotype in α6^{L9′S} mice, a nicotine dose-response relation was constructed. WT mice exhibited locomotor suppression at doses of nicotine between 0.5 and 2.0 mg/kg, i.p. (FIG. 1F), consistent with other reports. In contrast, α6^{L9′S} mice exhibited steadily increasing locomotor responses between 0.02 and 0.15 mg/kg, i.p. nicotine, followed by a decline at 0.4 mg/kg, i.p and locomotor suppression similar to WT mice at 1.5 mg/kg, i.p. Thus, selective activation of α6* receptors resulted in locomotor activation rather than locomotor suppression. This phenotype was not a stress response, as saline injections did not produce locomotor activation (FIG. 1G).

Locomotor activation in α6^{L9′S} mice was dependent on activation of nicotinic receptors; a strong block of the locomotor response in α6^{L9′S} (but not WT) mice pre-injected with mecamylamine (1 mg/kg, i.p.) was noted (FIG. 1H). Further, α6^{L9′S} locomotor activation acted through dopamine receptors; the response to 0.15 mg/kg, i.p. nicotine was completely inhibited by SCH23390 (D1DR antagonist; 2 mg/kg, i.p.) and partially inhibited by sulpiride (D2DR antagonist; 20 mg/kg, i.p.) (FIG. 1H).

To determine whether α6^{L9′S} mice develop tolerance or sensitization to nicotine psychomotor stimulation, groups of α6^{L9′S} mice were injected once daily for six days with nicotine. Injection of 0.02 mg/kg, i.p. or 0.08 mg/kg, i.p. nicotine did not produce any change in locomotor behavior after repeated injections (FIG. 1I). Nicotine also produces hypothermia in mice, and α4* hypersensitive mice exhibit this effect at nicotine doses ˜50-fold lower than WT mice. No difference was found between the hypothermia responses of WT and α6^{L9′S} mice.

Augmented Nicotine-Stimulated DA Release from Presynaptic Terminals in α6^{L9′S} Mice. Mouse α6* nAChRs expressed in midbrain DA neurons are located both on the cell body and on presynaptic terminals in caudate/putamen (CPu), nucleus accumbens (NAc), and striatal aspects of the olfactory tubercle (OT). Nicotine-stimulated [3H]DA release from striatal synaptosomes of WT and α6^{L9′S} mice was measured. Separate tissue samples containing striatum (ST; CPu and dorsal aspects of NAc) and olfactory tubercle (OT) were made. Because CPu receives DAergic projections mainly from substantia nigra whereas OT receives DAergic projections exclusively from VTA, this preparation crudely separates the mesostriatal and mesolimbic pathways. For total DA release in ST, there was no difference in Rₘₐₓ (FIG. 2A, Table 1), and a small but significant reduction in EC₅₀ for both transgenic lines relative to WT (FIG. 2G). In OT, there was a slight increase in Rₘₐₓ (FIG. 2D, Table 1) and a greater reduction in the EC₅₀ for α6^{L9′S} lines compared to WT (FIG. 2H). αCtxMII was used to inhibit α6* receptors, revealing the contribution of α6* and non-α6* nAChRs to this augmented DA release. In ST and OT, a significant increase in total DA release mediated by α6 at most nicotine concentrations was observed (FIGS. 2B and E). Interestingly, this was accompanied by a concomitant decrease in the non-α6* (α4β2* mediated) component (FIGS. 2C and F) in α6^{L9′S} samples. In ST and OT, a significant reduction in the EC₅₀ for the α6-dependent component was measured, and no change in EC₅₀ for the non-α6 component (FIGS. 2G and H). Overall, DA release from α6^{L9′S} striatal synaptosomes may be underestimated by this assay, as 20 mM K⁺ stimulated slightly less DA release in both α6^{L9′S} lines relative to WT mice (Table 1). These results directly reveal that selective α6* activation is capable of stimulating striatal DA release.

TABLE 1
DA release parameters for nicotine in tissues from WT and
α6^{L9′S} (striatum, ST and olfactory tubercle, OT)

| | Nicotine (ST) | WT | L9′S line 2 | L9′S line 5 |
|---|---|---|---|---|
| Total | EC50 (±SEM) | 0.36 ± 0.07 | 0.12 ± 0.02 | 0.17 ± 0.04 |
| | Rmax (±SEM) | 20.9 ± 1.0 | 21.4 ± 0.9 | 20.8 ± 1.2 |
| MII-sensitive | EC50 (±SEM) | 0.11 ± 0.04 | 0.047 ± 0.011 | 0.044 ± 0.011 |
| | Rmax (±SEM) | 5.41 ± 0.54 | 12.4 ± 0.6 | 9.56 ± 0.60 |
| MII-resistant | EC50 (±SEM) | 0.56 ± 0.12 | 0.47 ± 0.08 | 0.64 ± 0.11 |
| | Rmax (±SEM) | 15.6 ± 1.00 | 8.79 ± 0.43 | 11.4 ± 0.5 |
| 20 mM K⁺ | | 13.75 ± 0.24 | 10.76 ± 0.49 | 10.68 ± 0.43 |
| | Nicotine (OT) | WT | L9′S line 2 | L9′S line 5 |
| Total | EC50 (±SEM) | 0.31 ± 0.07 | 0.043 ± 0.006 | 0.064 ± 0.013 |
| | Rmax (±SEM) | 33.1 ± 2.1 | 36.0 ± 1.3 | 34.3 ± 1.7 |
| MII-sensitive | EC50 (±SEM) | 0.082 ± 0.037 | 0.025 ± 0.004 | 0.029 ± 0.008 |
| | Rmax (±SEM) | 7.03 ± 0.86 | 23.5 ± 1.0 | 19.7 ± 1.2 |
| MII-resistant | EC50 (±SEM) | 0.35 ± 0.10 | 0.40 ± 0.07 | 0.42 ± 0.14 |
| | Rmax (±SEM) | 23.7 ± 2.0 | 17.1 ± 0.9 | 17.0 ± 1.7 |
| 20 mM K⁺ | | 16.62 ± 0.96 | 14.43 ± 1.16 | 14.62 ± 1.31 |

α4β2* nAChRs modulate striatal GABA release. GABA release from striatal (ST and OT combined) synaptosomes of WT and α6^{L9′S} mice was measured to determine whether any α6*-dependent component was revealed by the gain-of-function L9′S mutation. There was no difference in total GABA release for any genotype comparison (FIG. 2I, Table 2), and there was no α6* component (FIGS. 2J and K, Table 2). A students t-test on Rₘₐₓ and EC₅₀ values revealed no significant difference between total and αCtxMII-resistant GABA release for any genotype (WT Rₘₐₓ p=0.47, WT EC₅₀ p=0.73; line 2 Rₘₐₓ p=0.93, line 2 EC₅₀ p=0.31; line 5 Rₘₐₓ p=0.62, line 5 EC₅₀ p=0.63). Furthermore, there was no significant difference between any genotype on Rₘₐₓ or EC₅₀ (2-way ANOVA with Tukey post-hoc comparison −Rₘₐₓ F(2,66)=1.87, p=0.163; EC₅₀ F_{(2,66)}=0.744, p=0.48) nor was there any effect of αCtxMII across all genotypes on Rₘₐₓ or EC₅₀ (Rₘₐₓ F_{(1,66)}=0.35, p=0.554; EC₅₀ F_{(1,66)}=0.643, p=0.426). Stimulation with 20 mM K⁺ showed no differences between WT and α6^{L9′S} sample for GABA release (Table 2). This indicated that GABAergic terminals in striatum, either derived locally or from VTA or SNr GABAergic neurons, contain no appreciable α6* nAChRs.

TABLE 2
GABA release parameters for nicotine in tissues from WT and
α6^{L9′S} (striatum, ST and olfactory tubercle, OT)

| | Nicotine | | | |
|---|---|---|---|---|
| | (ST/OT) | WT | L9′S line 2 | L9′S line 5 |
| Total | EC50 (±SEM) | 1.30 ± 0.32 | 1.53 ± 0.44 | 2.47 ± 0.81 |
| | Rmax (±SEM) | 1.64 ± 0.32 | 1.25 ± 0.06 | 1.22 ± 0.11 |
| MII-sensitive | EC50 (±SEM) | no activity | no activity | no activity |
| | Rmax (±SEM) | no activity | no activity | no activity |
| MII-resistant | EC50 (±SEM) | 1.50 ± 0.47 | 4.18 ± 2.54 | 2.00 ± 0.54 |
| | Rmax (±SEM) | 1.40 ± 0.08 | 1.26 ± 0.08 | 1.30 ± 0.10 |
| 20 mM K⁺ | | 8.31 ± 0.34 | 8.24 ± 0.45 | 8.43 ± 0.50 |

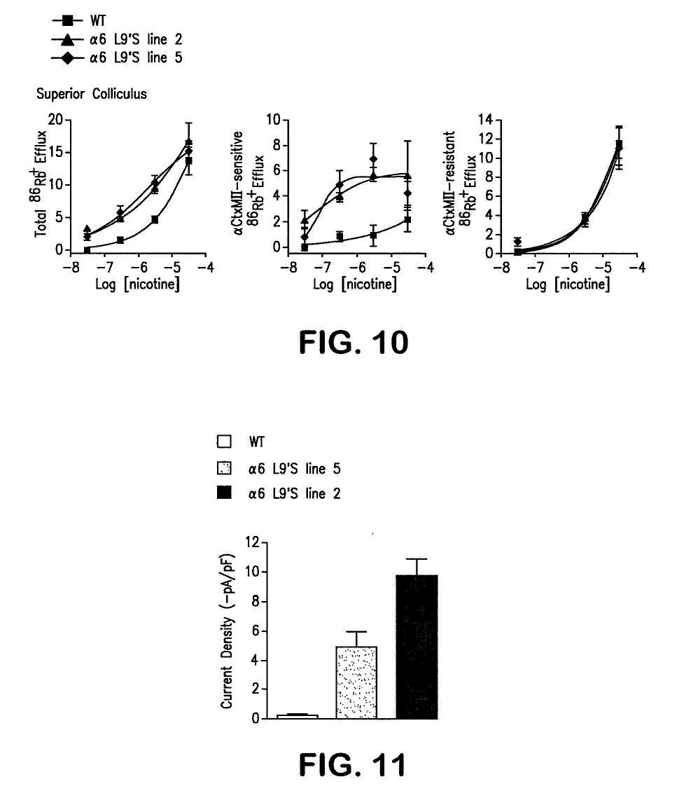

α6* receptors synthesized in retinal ganglion cells reside in the superficial layers of superior colliculus (SC) and thus, nicotine-stimulated ⁸⁶Rb⁺ efflux from SC synaptosomes was measured. α6^{L9′S} SC αCtxMII-sensitive receptors were hypersensitive to nicotine relative to WT, whereas αCtxMII-resistant ⁸⁶Rb⁺ efflux was unchanged (FIG. 10).

DA Release and Locomotor Activity are Precisely Controlled by Varying α6* Agonist Efficacy. The DA release data suggested a mechanism involving dopamine for the psychomotor stimulant action of nicotine, as well as the spontaneous hyperactivity observed. For DA release in WT mice, TC 2429 (Bhatti et al., 2008) (FIG. 3A) is a full agonist (vs. nicotine) with 3-fold selectivity at α6β2* and a weak partial agonist at α4β2, whereas TC 2403 (Bencherif et al., 1996; Lippiello et al., 1996) (FIG. 3E) is a full agonist (vs. nicotine) at α4β2 and has no activity at α6β2* (Table 3). TC 2429, like nicotine, was more efficacious and more potent for DA release from striatal synaptosomes of α6^{L9′S} mice relative to WT (FIG. 3B). In both α6^{L9′S} lines, Rₘₐₓ was greater, and the EC₅₀ was reduced relative to WT. This was entirely attributable to α6* nAChRs (FIG. 3C), which accounted for a greater proportion of the total response. There was a concomitant decline in the total response, but not the EC₅₀, for α4β2* (αCtxMIIresistant) nAChRs (FIG. 3D). We characterized the ability of TC 2429 to induce psychomotor activation in α6^{L9′S} and WT control mice. Similar to nicotine, injections of TC 2429 stimulated locomotor activity in α6^{L9′S} mice but not WT (FIG. 3I). Unlike nicotine, TC 2429 did not produce locomotor suppression in WT mice.

TABLE 3
DA release parameters for TC 2429 and TC2403 in tissues from
WT and α6^{L9′S} (striatum, ST and olfactory tubercle, OT)

| | TC 2429 (ST/OT) | WT | L9′S line 2 | L9′S line 5 |
|---|---|---|---|---|
| Total | EC50 (±SEM) | 0.0093 ± 0.005 | 0.0022 ± 0.0003 | 0.0026 ± 0.0004 |
| | Rmax (±SEM) | 8.68 ± 1.25 | 12.87 ± 0.37 | 11.86 ± 0.41 |
| | 10 μM nic | 17.70 | 17.30 | 16.70 |
| | %10 μM nic | 49.0 | 73.40 | 71.00 |
| MII-sensitive | EC50 (±SEM) | 0.0058 ± 0.0035 | 0.0016 ± 0.00014 | 0.0019 ± 0.0003 |
| | Rmax (±SEM) | 4.29 ± 0.44 | 10.4 ± 0.14 | 9.11 ± 0.25 |
| | 10 μM nic | 3.19 | 8.53 | 5.97 |
| | %10 μM nic | 134.60 | 122.00 | 152.70 |
| MII-resistant | EC50 (±SEM) | 0.014 ± 0.006 | 0.01 ± 0.003 | 0.011 ± 0.0039 |
| | Rmax (±SEM) | 4.39 ± 0.32 | 2.64 ± 0.14 | 3.07 ± 0.19 |
| | 10 μM nic | 14.55 | 8.75 | 10.78 |
| | %10 μM nic | 30.24 | 30.17 | 28.48 |

| | TC 2403 (ST/OT) | WT | L9′S line 2 | L9′S line 5 |
|---|---|---|---|---|
| Total | EC50 (±SEM) | 2.36 ± 0.56 | 0.70 ± 0.15 | 0.82 ± 0.22 |
| | Rmax (±SEM) | 12.37 ± 0.93 | 10.72 ± 0.61 | 10.57 ± 0.74 |
| | 10 μM nic | 16.69 | 19.68 | 20.65 |
| | %10 μM nic | 74.11 | 54.47 | 51.19 |
| MII-sensitive | EC50 (±SEM) | 0.06 ± 0.18 | 0.19 ± 0.04 | 0.1 ± 0.063 |
| | Rmax (±SEM) | 0.47 ± 0.2 | 4.83 ± 0.15 | 3.43 ± 0.32 |
| | 10 μM nic | 1.63 | 10.77 | 10.39 |
| | %10 μM nic | 28.83 | 44.85 | 33.01 |
| MII-resistant | EC50 (±SEM) | 2.85 ± 0.35 | 3.53 ± 0.83 | 4.03 ± 0.84 |
| | Rmax (±SEM) | 12.32 ± 0.26 | 7.26 ± 0.29 | 9.3 ± 0.33 |
| | 10 μM nic | 15.07 | 8.91 | 10.26 |
| | %10 μM nic | 81.78 | 81.48 | 90.64 |

TC 2403 was slightly more potent and had equivalent efficacy for DA release in α6^{L9′S} versus WT striatum (FIG. 3F). The increased potency was due to the amplification of an αCtxMII-sensitive response not visible in WT tissue preparations (FIG. 3G). TC 2403 was a partial agonist at this receptor (Table 3). Consistent with competition between α6 and α4 subunits for common β2 subunits, there was a decline in Rₘₐₓ in α6^{L9′S} mice for α4β2* (αCtxMII-resistant) nAChRs (FIG. 3H and Table 3). In locomotor assays, TC 2403 induced a slight locomotor activation in α6^{L9′S} but not WT mice (FIG. 3J), consistent with partial activity at α6* nAChRs. Nicotine (10 μM) was used as a positive control for DA release for TC 2429 and TC 2403 (Table 3). For nicotine, TC 2429 and TC 2403, we noted a tight correlation between α6*-dependent DA release in vitro and peak locomotor activity in vivo (FIG. 3K). While not wishing to be bound by any particular theory, these results suggested a mechanism whereby agonism at α6* nAChRs stimulates striatal DA release and produces locomotor stimulation, perhaps without GABAergic attenuation which is normally co-activated by α4β2* activation (FIG. 2I-K).

α6^{L9′S} Receptors Sensitize DA Neurons to Activation by Nicotine. To directly determine whether DA neurons in α6^{L9′S} mice express hypersensitive α6* nAChRs, coronal midbrain slices were prepared (FIG. 4A) and patch-clamp recordings from VTA DA neurons were made in whole-cell configuration. DA neurons can be identified based on expression of tyrosine hydroxylase (TH) (FIG. 4B), and with electrophysiology; DA neurons exhibit pacemaker firing (1-5 Hz), membrane potential adaptations in response to hyperpolarizing current injections, and often express Iₕ. Currents induced by local nicotine application (FIG. 4C) at a range of concentrations were recorded. Nicotinic currents in α6^{L9′S} neurons were markedly hypersensitive to nicotine; puffs of nicotine (1 μM) elicited inward currents that were larger than those seen in WT neurons at any concentration (FIG. 4D). At all nicotine concentrations, α6_{L9′S} neurons of both transgenic lines showed larger average peak current responses than WT neurons (FIG. 4E). Normalizing the peak current amplitude to cell capacitance yielded identical results (FIG. 11).

Whole-cell responses to 1 μM nicotine were >90% blocked when αCtxMII (100 nM) was added to the perfusate (FIG. 4F, upper panel). Consistent with other reports (McIntosh et al., Mol Pharmacol 65, 944-952, 2004), αCtxMII block persisted 30 min after washout. Likewise, responses to 1 μM nicotine were 100% blocked in the presence of dihydro-β-erythroidine (DHβE, 2 μM), a potent inhibitor of most β2* receptors (FIG. 4F, lower panel). These results indicated that hypersensitive nicotinic receptors in α6_{L9′S} VTA DA neurons contain α6 and β2 subunits, which concurs with functional measurements in striatal synaptosomes. Although the general kinetics of these hypersensitive responses suggests that α6^{L9′S} receptors are post-synaptic, a presynaptic mechanism is not excluded by the data. To address this AMPA/Kainate receptors were inhibited with 6-cyano-7-nitroquinoxaline-2,3-dione (CNQX, 15 μM) and voltage gated Na⁺ channels with tetrodotoxin (TTX, 0.5 μM). Inward current responses to 1 μM nicotine were unaffected by either CNQX or TTX (FIG. 4G). Furthermore, no effect was found (n=6, 0% block) on peak current amplitude (1 μM nicotine) in the presence of methyllycaconitine (MLA, 10 nM) (FIG. 4G). Thus, hypersensitive α6* responses were consistent with a post-synaptic mechanism.

VTA DA neuron pacemaker firing was unaltered in WT versus α6^{L9′S} slices (FIG. 4I). To determine whether hypersensitive nicotinic receptors in α6_{L9′S} neurons were capable of acutely altering the excitability of VTA DA neurons, action potential firing in response to local nicotine application was recorded. Nicotine (1 μM) induced a transient increase in the instantaneous firing rate and a depolarization of the membrane potential (FIG. 4H). There was a significant increase in the firing rate for α6_{L9′S}, but not WT, cells (FIG. 4I).

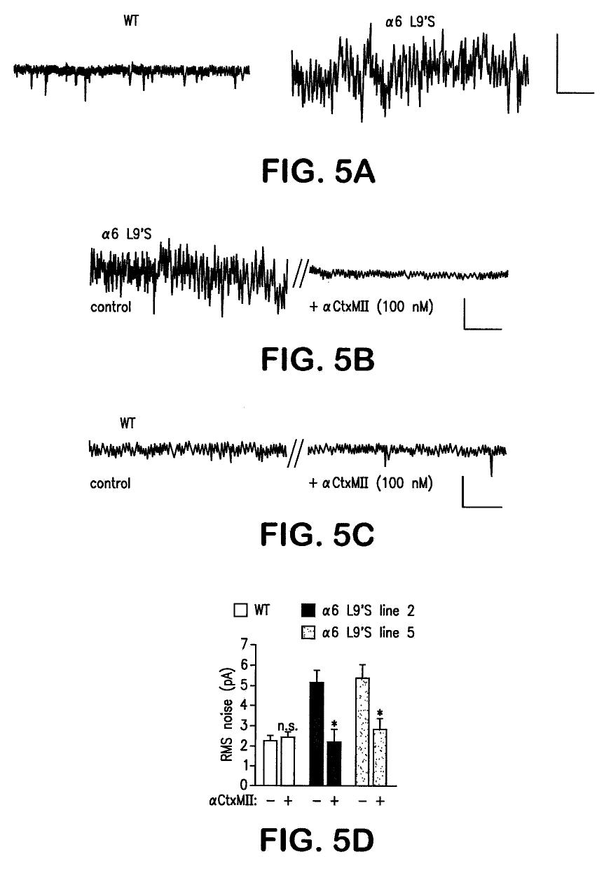

Greater fluctuations were noticed in the holding current in α6^{L9′S} VTA DA neurons than in WT (FIG. 5A). This suggests that some α6^{L9′S} channels are tonically active, reminiscent of previous observations in α4 nAChR L9′S and L9′A knock-in mice (Labarca et al., 2001; Shao et al., 2008). To determine whether this change in resting membrane conductance was due to active α6^{L9′S} channels, α6* receptors, were blocked by application of αCtxMII. α6 blockade completely eliminated this increased channel noise in all α6^{L9′S} cells tested (FIG. 5B). WT neurons bathed in αCtxMII served as a control (FIG. 5C). To quantify this, the root-mean-square (RMS) value of the fluctuations in WT and α6^{L9′S} voltage clamp recordings eas measured. There was a significant decrease in the noise for α6^{L9′S} cells in the presence of αCtxMII (FIG. 5D). No effect of CNQX, picrotoxin, or TTX on the increased membrane noise in α6^{L9′S} cells was found (FIG. 12). DHβE, however, completely eliminated the increased noise similar to αCtxMII (FIG. 12). These results further demonstrated that VTA DA neurons express functional, hypersensitive α6* nAChRs, and that these receptors may be activated by local ACh.

The properties of hypersensitive α6* receptors in DA neurons of the substantia nigra pars compacta (SNc) were examined and found to have similar results to the VTA. SNc DA neurons from α6^{L9′S} mice expressed hypersensitive nicotinic currents relative to WT, and firing rates were excited by 1 μM nicotine in α6^{L9′S} but not WT cells (FIGS. 13A and B). Finally, α6^{L9′S} SNc DA neurons expressed tonically active, αCtxMII-sensitive α6* receptors (FIG. 13C).

Specific Expression of Functional α6* Receptors in Midbrain DA Neurons. Although expression data suggest selective expression of α6* nAChRs in DA neurons, no electrophysiological experiments supporting this idea have been published. Midbrain DA neurons typically express D2-class autoreceptors, in contrast to midbrain GABAergic neurons. Electrophysiological recordings were taken of 15 VTA neurons, ten of which expressed α6* nAChRs (based on large inward current responses to 1 μM nicotine), and five which did not. All cells expressing α6* nAChRs were sensitive to the inhibitory properties of the D2 DA receptor agonist quinpirole (FIG. 6A, panel i), indicating that these cells were likely DAergic. In contrast, most α6*-negative cells did not express D2 receptors (FIG. 6A, panel ii). To more accurately determine whether α6* receptors are functionally expressed in DA and/or GABA cells in midbrain, recordings were taken from substantia nigra (SN) neurons in slices from WT and α6^{L9′S} mice. The spatial partitioning of DA and GABA neurons in the SN pars compacta (SNc) and pars reticulata (SNr) (FIG. 6B, panel i) was combined with electrophysiological signatures (FIG. 6B, panel ii-iv; see Experimental Procedures) to unambiguously identify these cell types. In whole-cell recordings from α6_{L9′S} and WT SN neurons, hypersensitive nicotinic responses were observed in α6^{L9′S} DA neurons, but not in GABA neurons (FIG. 6C). Average peak current amplitudes were comparable between α6^{L9′S} line 2 and 5 in SNc DA neurons (FIG. 6D, panel i). Average responses in WT and α6^{L9′S} GABA neurons were <10 pA (FIG. 6D, panel ii). Responses to nicotine at 1 μM were undetectable in WT DA and GABA neurons (FIG. 6C, panel i), however, these cells responded predictably to 100 μM or 1 mM nicotine (data not shown). As a control for the specificity of these results, recordings were taken from SNc and SNr neurons in slices from α4^{L9′A} mice. Hypersensitive nicotinic responses were found in SNc and SNr neurons from these mice (FIGS. 6C and D), consistent with the idea that α4 is expressed in both DA and GABA cells. These results, coupled with the absence of α6* receptors in GABAergic presynaptic terminals in striatum (FIG. 2I-K), indicated that functional α6* receptors, in contrast to α4* receptors, are restricted to DA neurons in the midbrain.

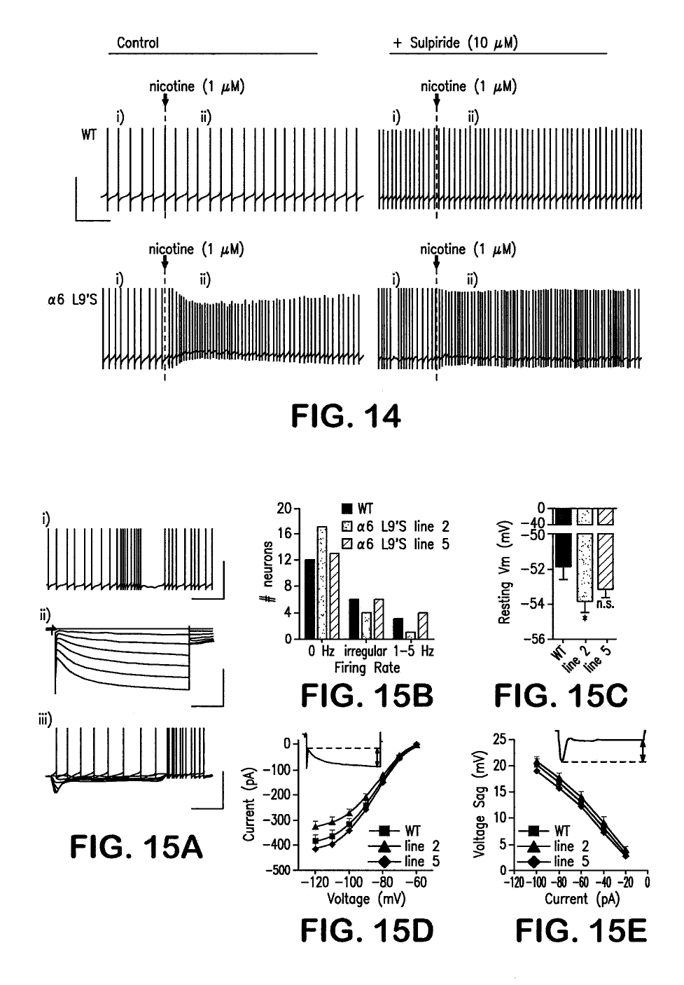

The results thus far suggested increased DA tone in midbrain and/or striatum. To examine this, pacemaker and nicotine-induced firing of VTA DA neurons were studied in the absence and presence of sulpiride, a D2 DA receptor antagonist. There was no change in the ability of sulpiride to modestly increase baseline firing between WT and α6^{L9′S} VTA DA cells (FIG. 14). Further, sulpiride did not affect nicotine-induced increases in firing in α6^{L9′S} cells or its lack of effect in WT cells (FIG. 14). To determine whether augmented striatal DA release in α6^{L9′S} mice (FIG. 2) could be detected in brain slices using patch-clamp recordings, striatal cholinergic interneurons were studied. These cells were easily identifiable (FIG. 15A), their activity is modulated by DA, and they are the source of ACh that activates α6* receptors on presynaptic DA terminals. No change was detected in spontaneous firing between WT and α6^{L9′S} cells (FIG. 15B). The resting membrane potential for α6^{L9′S} line 2 (but not line 5) was hyperpolarized compared to WT (FIG. 15C). Although DA may modulate Iₕ currents in cholinergic interneurons (Deng et al., 2007), no difference was found between WT and α6^{L9′S} Iₕ expression or function in these cells (FIGS. 15D and E).

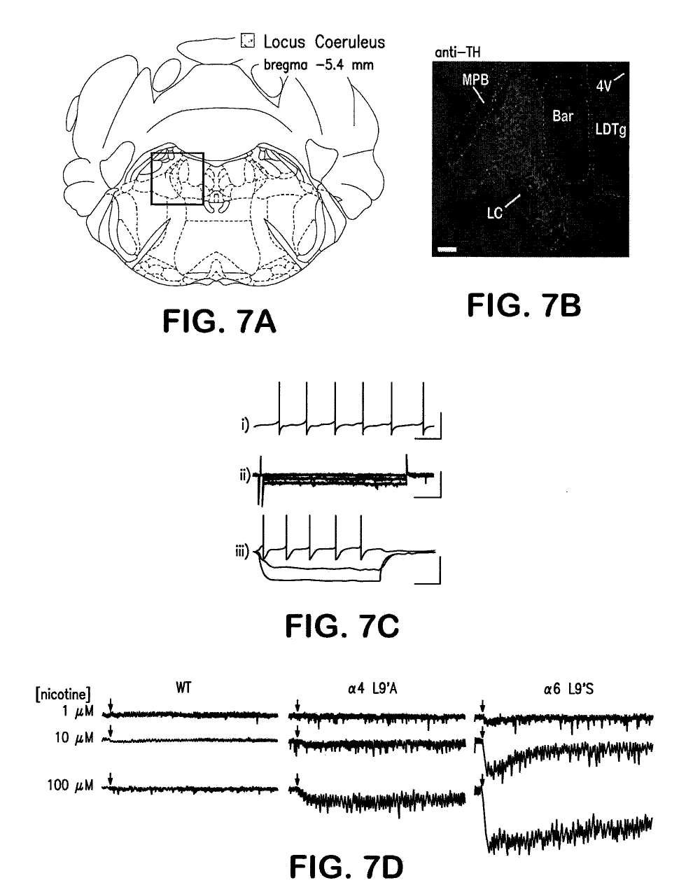

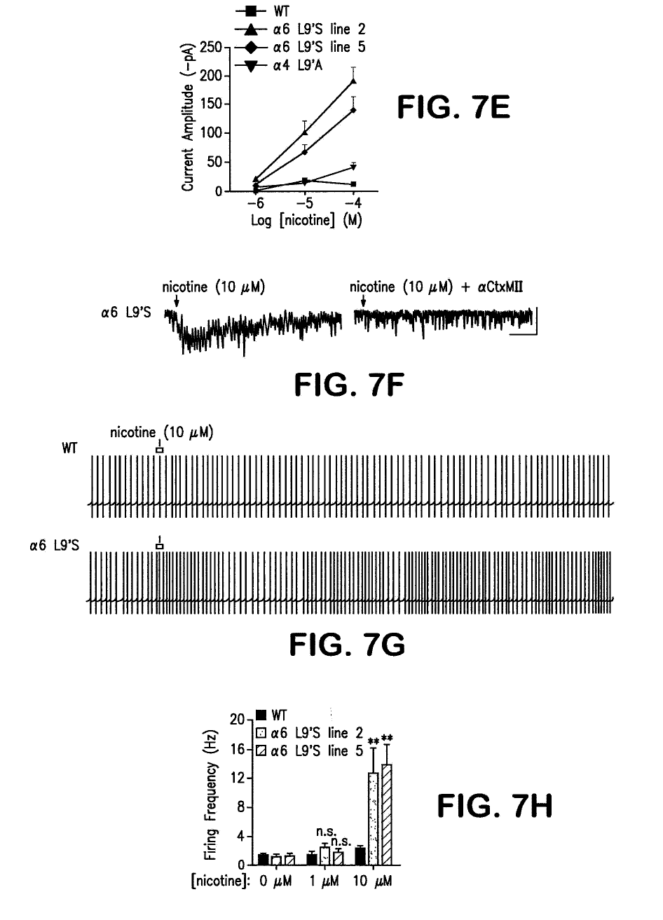

α6^{L9′S} nAChRs in Locus Coeruleus Neurons Cannot Account for Behavioral Phenotypes in Mutant Mice. α6* nAChR subunits are expressed in locus coeruleus (LC). To determine whether α6* activation in α6^{L9′S} mice might also stimulate LC neuron firing, recording were taken from LC neurons in coronal slices from WT and α6^{L9′S} mice (FIG. 7A). LC neurons express tyrosine hydroxylase (TH) (FIG. 7B), exhibited spontaneous firing (1-2 Hz; FIG. 7C, panel i) and lack Iₕ currents (FIG. 7C, panel ii and iii). LC neurons from α6^{L9′S} mice exhibited larger responses to locally applied nicotine compared to WT cells and cells from α4^{L9′A} knock-in mice (FIGS. 7D and E). Although these responses were sensitive to αCtxMII (FIG. 7F) and therefore α6-dependent, they were smaller and approximately 10-fold less sensitive to nicotine than receptors on VTA DA neurons (compare FIGS. 4E and 7E). LC neurons were also able to fire action potentials in response to nicotine (FIG. 7G), but at concentrations of nicotine 10-fold higher than for DA neurons (FIG. 7H). The reduced sensitivity of LC α6* nAChRs relative to receptors on DA neurons suggests that they do not participate in the psychostimulant response to nicotine we observed. In support of this, no change was found in the locomotor response to nicotine when prazosin (α1AR antagonist) or yohimbine (α2AR antagonist) were administered prior to challenge with nicotine (FIG. 1H).

### EXAMPLE 2

In the present study, the cell biological and biophysical properties of receptors containing α6 and β3 subunits were examined by using fluorescent proteins fused within the M3-M4 intracellular loop. Receptors containing fluorescently tagged β3 subunits were fully functional compared with receptors with untagged β3 subunits. It was found that β3- and α6-containing receptors were highly expressed in neurons and that they colocalized with coexpressed, fluorescent α4 and β2 subunits in neuronal soma and dendrites. Forster resonance energy transfer (FRET) revealed efficient, specific assembly of β3 and α6 into nicotinic receptor pentamers of various subunit compositions. Using FRET, it was directly demonstrate that only a single β3 subunit is incorporated into nicotinic acetylcholine receptors (nAChRs) containing this subunit, whereas multiple subunit stoichiometries exist for α4- and α6-containing receptors. Finally, it was demonstrated that nicotinic ACh receptors are localized in distinct microdomains at or near the plasma membrane using total internal reflection fluorescence (TIRF) microscopy. While not wishing to be bound by any particular theory, it is proposed that neurons contain large, intracellular pools of assembled, functional nicotinic receptors, which may provide them with the ability to rapidly up-regulate nicotinic responses to endogenous ligands such as ACh, or to exogenous agents such as nicotine. Finally, this report is the first to directly measure nAChR subunit stoichiometry using FRET and plasma membrane localization of α6- and β3-containing receptors using TIRF.

Reagents. Unless otherwise noted, all chemicals were from Sigma-Aldrich (St. Louis, Mo.). DNA oligonucleotides for PCR and site-directed mutagenesis were synthesized by Integrated DNA Technologies, Inc. (Coralville, Iowa). Restriction enzymes for molecular biology were purchased from Roche Diagnostics (Indianapolis, Ind.) or New England Biolabs (Ipswich, Mass.). Glass-bottomed dishes (35 mm) coated with L-polylysine were purchased from MatTek (Ashland, Mass.).

Cell Culture and Transfection. N2a cells (American Type Culture Collection, Manassas, Va.) were maintained in Dulbecco's modified Eagle's medium (high glucose with 4 mM L-glutamine; Invitrogen, Carlsbad, Calif.)/OPTI-MEM (Invitrogen) mixed at a ratio of 1:1 and supplemented with 10% fetal bovine serum (Invitrogen), penicillin (Mediatech, Herndon, Va.), and streptomycin (Invitrogen). N2a cells were transfected in DMEM without serum or antibiotics. Transfection was carried out using LIPOFECTAMINE/PLUS (Invitrogen) according to the manufacturer's instructions and with the following modifications. For a 35-mm dish, 1 to 2 μg of total plasmid DNA was mixed with 100 μl of DMEM and 6 μl of PLUS reagent. DMEM/DNA was combined with a mixture of 100 μl of DMEM and 4 μl of Lipofectamine reagent. Rat hippocampal neurons were dissociated and plated on glass-bottomed imaging dishes as described previously (Slimko et al., 2002). For primary neuron transfection, Lipofectamine 2000 (Invitrogen) was used in conjunction with Nupherin (BIOMOL Research Laboratories, Plymouth Meeting, Pa.) as described below. In brief, in total 1 μg of DNA was incubated with 20 μg of Nupherin in 400 μl of Neurobasal medium without phenol red (Invitrogen), whereas 10 μl of LIPOFECTAMINE 2000 was mixed in 400 μl of Neurobasal medium (Invitrogen). After 15 min, the two solutions were combined and incubated for 45 min. Neuronal cultures in 35-mm glass-bottomed culture dishes were incubated in the resulting 800-e1 mixture for 120 min, followed by removal of transfection media and refeeding of the original, pretransfection culture media.

Plasmids and Molecular Biology. Mouse α4 and β2 nAChR cDNAs in pCI-neo, both untagged and modified with YFP or CFP fluorescent tags, have been described previously (Nashmi et al., 2003). A full-length mouse α6 I.M.A.G.E. cDNA (ID no. 4501558) was obtained from Open Biosystems (Huntsville, Ala.). A modified α6 cDNA was constructed that 1) lacked the 5′ and 3′ untranslated regions and 2) contained a Kozak sequence (GCC ACC) before the ATG start codon to facilitate efficient translation initiation. Rat β4 was cloned into pAMV. pEYFP—N1 and pECFP—N1 (Clontech, Mountain View, Calif.) were used to construct fluorescent nAChR cDNAs. A QUICK-CHANGE (Stratagene, La Jolla, Calif.) kit was used to construct β3 (WT or XFP-modified) cDNAs containing a V13′S point mutation.

To design fluorescently labeled α6 and β3 subunits, the XFP moiety was inserted into the M3-M4 loop of each subunit. It was previously found that this region is appropriate for insertion in nAChR α4 and β2 subunits (Nashmi et al., J. Neurosci 27:8202-18, 2003), the nAChR ysubunit, and GluCl α and β subunits. The XFP moiety was inserted into the M3-M4 loop at positions that avoided the conserved amphipathic a-helix and putative cell sorting motifs and phosphorylation sites (FIG. 17, A and B). To construct nAChRs with XFP inserted into the M3-M4 loop, a two-step PCR protocol was used. First, YFP or CFP was amplified with PCR using oligonucleotides designed to engineer 5′ and 3′ overhangs of 15 base pairs that were identical to the site where XFP was to be inserted, in frame, into the nAChR M3-M4 loop. A Gly-Ala-Gly flexible linker was engineered between the nAChR sequence and the sequence for YFP/CFP at both the 5′ and 3′ ends. In the second PCR step, 100 ng of the first PCR reaction was used as a primer pair in a modified QUICKCHANGE reaction using Pfu Ultra II (Stratagene, Cedar Creek, Tex.) polymerase and the appropriate nAChR cDNA as a template. All DNA constructs were confirmed with sequencing and, in some cases, restriction mapping.

cRNA for injection and expression in X. laevis oocytes was prepared using a T7 or Sβ6 in vitro transcription kit (mMessage mMachine; Ambion, Foster City, Calif.) according to the manufacturer's instructions. RNA yield was quantified with absorbance at 260 nm. RNA quality was assessed by observing absorbance profiles across a range of wavelengths between 220 and 320 nm. Spectrophotometric analysis was performed using a ND-1000 spectrophotometer (Nano-Drop, Wilmington, Del.).

Confocal Microscopy. N2a cells were plated on 35-mm glass-bottomed dishes, transfected with nAChR cDNAs, and they were imaged live 24-48 h after transfection. X. laevis oocytes were imaged 3 days after RNA injection. Oocytes were placed in an imaging chamber and allowed to settle for 20 min before imaging. To eliminate autofluorescence, growth medium was replaced with an extracellular solution containing the following components: 150 mMNaCl, 4 mM KCl, 2 mM CaCl2, 2 mM MgCl2, 10 mM HEPES, and 10 mM D-glucose, pH 7.4. Cells were imaged with a Nikon (Nikon Instruments, Melville, N.Y.) C1 laser-scanning confocal microscope system equipped with spectral imaging capabilities and a Prior (Rockland, Me.) remote-focus device. For oocytes, a Nikon Plan Apo 20×0.75 numerical aperture (NA) air objective was used, whereas a Nikon Plan Apo 60×1.40 NA oil objective was used for mammalian tissue culture cells. Pinhole diameter was 30-60 μm, and cells were imaged at 12-bit intensity resolution over 512×512 pixels at a pixel dwell time of 4 to 6 μs. CFP was excited using a 439.5-nm modulated diode laser, and YFP was excited with an argon laser at 514.5-nm. In most cases, imaging was carried out using the Nikon C1si DEES grating and spectral detector with 32 parallel photomultiplier tubes. This allowed collection of spectral images (λ stacks). In such images, each pixel of the X-Y image contains a list of emission intensity values across a range of wavelengths. Light was collected between 450 and 600 nm at a bandwidth of 5 nm. The 515-nm channel was intentionally blocked while we used the 514.5-nm laser for YFP bleaching. Because the emission profile of YFP and CFP significantly overlap, the Nikon EZC1 linear unmixing algorithm was used to reconstruct YFP and CFP images. Experimental spectral images with both YFP and CFP-labeled nAChR subunits were unmixed using reference spectra from images with only YFP- or CFP-labeled nAChR subunits. For each pixel of a spectral image, intensity of YFP and CFP was determined from fluorescence intensity values at the peak emission wavelength derived from the reference spectra.

Spectral FRET Analysis. To examine FRET between various nAChR subunits, the acceptor photobleaching method was used with a modified fluorescence recovery after photobleaching macro built into the Nikon EZC1 imaging software. In this method, FRET was detected by recording CFP dequenching during incremental photodestruction of YFP. A spectral image was acquired once before YFP bleaching and at six time points every 10 s during YFP bleaching at 514.5 nm. Laser power during bleaching varied from cell to cell, but was between 25 and 50%. One bleach scan per cycle was used. This bleaching protocol was optimized to achieve 70 to 80% photodestruction of YFP while still enabling us to record incremental increases in CFP emission at each time point. In the confocal microscope, nAChRs labeled with XFP usually exhibit a uniform, intracellular distribution, regardless of the subunit being examined. To measure FRET, spectral images were unmixed into their CFP and YFP components as described above. Little or no difference was found in FRET for various cellular structures or organelles in N2a cells, and we measured CFP and YFP mean intensity throughout the entire cell by selecting the cell perimeter as the boundary of a region of interest in Nikon's EZC1 software. CFP and YFP components were saved in Excel format, and fluorescence intensities were normalized to the prebleach time point (100%). FRET efficiency (E) was calculated as E=1−(I_{DA}/I_{D}), where I_{DA} represents the normalized fluorescence intensity of CFP (100%) in the presence of both donor (CFP) and acceptor (YFP), and I_{D} represents the normalized fluorescence intensity of CFP in the presence of donor only (complete photodestruction of YFP). The I_{D} value was extrapolated from a scatter plot of the fractional increase of CFP versus the fractional decrease of YFP. The E values were averaged from several cells per condition. Data are reported as mean±S.E.M.

TIRF Microscopy. N2a cells cultured in glass-bottomed, polyethylenimine-coated imaging dishes were transfected with cDNA mixtures as described above. Cells, superfused with the same imaging solution used for confocal microscopy, were imaged 18 to 24 h after transfection to minimize overexpression artifacts. TIRF images were obtained with an inverted microscope (Olympus IX71; Olympus America, Inc., Center Valley, Pa.) equipped with a 488-nm air-cooled argon laser (P/N IMA111040ALS; Melles Griot, Carlsbad, Calif.). Laser output was controlled with a UNIBLITZ shutter system and drive unit (P/N VMM-D1; Vincent Associates, Rochester, N.Y.) equipped with a Mitutoyo (Mitutoyo America, City of Industry, CA) micrometer to control TIRF evanescent field illumination. TIRF imaging was carried out with an Olympus PlanApo 100×1.45 NA oil objective, and images were captured with a 16-bit resolution Photometrics Cascade charge-coupled device camera (Photometrics, Tucson, Ariz.) controlled by SlideBook 4.0 imaging software (Intelligent Imaging Innovations, Santa Monica, Calif.).

Two-Electrode Voltage-Clamp Electrophysiology. Stage V to VI X. laevis oocytes were isolated as described previously (Quick and Lester, Ion Channels of Excitable Cells, Narahashi T ed., pp 261-279, 1994). Stock RNAs were diluted into diethyl pyrocarbonatetreated water and injected 1 day after isolation. RNA was injected in a final volume of 50 nl per oocyte using a digital microdispenser (Drummond Scientific, Broomall, Pa.). After injection, oocytes were incubated in ND-96 solution (96 mM NaCl, 2 mM KCl, 1 mM CaCl2, 1 mM MgCl2, and 5 mM HEPES/NaOH, pH 7.6) supplemented with 50 μg/ml gentamicin and 2.5 mM sodium pyruvate. After 1 to 4 days for nAChR expression, oocytes were used for recording or confocal microscopy.

Agonist-activated nicotinic receptor responses were measured by two-electrode voltage-clamp recording using a GeneClamp 500 (Molecular Devices, Sunnyvale, Calif.) voltage clamp. Electrodes were constructed from Kwik-Fil borosilicate glass capillary tubes (1B150F-4; WPI, Sarasota, Fla.) using a programmable microelectrode puller (P-87; Sutter Instrument Company, Novato, Calif.). The electrodes had tip resistances of 0.8 to 2.0 MΩ after filling with 3 M KCl. During recording, oocytes were superfused with Ca²⁺-free ND-96 via bath application and laminar-flow microperfusion using a computer-controlled application and washout system (SF-77B; Warner Instruments, Hamden, Conn.) (Drenan et al., 2005). The holding potential was −50 mV, and ACh was diluted in Ca²⁺-free ND-96 and applied to the oocyte for 2 to 10 s followed by rapid washout. Data were sampled at 200 Hz and low-pass filtered at 10 Hz using the GeneClamp 500 internal low-pass filter. Membrane currents from voltage-clamped oocytes were digitized (Digidata 1200 acquisition system; Molecular Devices) and stored on a PC running pCLAMP 9.2 software (Molecular Devices). Concentration-response curves were constructed by recording nicotinic responses to a range of agonist concentrations (six to nine doses) and for a minimum of six oocytes. EC₅₀ and Hill coefficient values were obtained by fitting the concentration-response data to the Hill equation. All data were reported as mean±S.E.M.

Whole-Cell Patch-Clamp Electrophysiology. N2a cells expressing YFP-labeled nicotinic receptors were visualized with an inverted microscope (Olympus IMT-2, DPlan 10×0.25 NA and MPlan 60×0.70 NA) under fluorescence illumination (mercury lamp). Patch electrodes (3-6 MΩ) were filled with pipette solutions containing 88 mM KH2PO4, 4.5 mM MgCl2, 0.9 mM EGTA, 9 mM HEPES, 0.4 mM CaCl2, 14 mM creatine phosphate (Tris salt), 4 mM Mg-ATP, and 0.3 mM GTP (Tris salt), pH 7.4 with KOH. The extracellular solution was 150 mM NaCl, 4 mM KCl, 2 mM CaCl2, 2 mM MgCl2, 10 mM HEPES, and 10 mM D-glucose, pH 7.4. Standard whole-cell recordings were made using an Axopatch 1-D amplifier (Molecular Devices), low-pass filtered at 2 to 5 kHz, and digitized online at 20 kHz (pClamp 9.2; Molecular Devices). Series resistance was compensated 80%, and the membrane potential was held at −70 mV. Recorded potentials were corrected for junction potential.

ACh was delivered using a two-barrel glass O-shaped tube (outer diameter ˜200 μm; pulled from 1.5-mm-diameter θ-shaped borosilicate tubing) connected to a piezoelectric translator (LSS-3100, Burleigh Instruments, Fishers, N.Y.). ACh was applied for 500 ms (triggered by pCLAMP 9.2), and solution exchange rates measured from open tip junction potential changes during application with 10% extracellular solution were typically ˜300 μs (10-90% peak time). Data were reported as mean±S.E.M. for the peak current response to 1 μM ACh, and statistical significance was determined using a Wilcoxon signed rank test.

Design and Construction of α6 and β3×FP Fusions. XFP fusions were inserted into the M3-M4 loop of mouse α6 and β3 nAChR subunits. Like all members of the Cys-loop family, α6 and β3 have predicted c-helices at the N and C-terminal ends of their M3-M4 loop (FIG. 17, A and B) that may be important in ion permeation. In addition to avoiding these regions, potential phosphorylation sites and trafficking motifs were also avoided (FIG. 17, A and B). The XFP fusion cassette also consisted of a Gly-Ala-Gly flexible linker flanking the XFP open reading frame on both the N- and C-terminal side. Three independent XFP fusions were built for α6 and two XFP fusions for β3 (FIG. 17C). These were designated according to the residue immediately N-terminal to the beginning of the Gly-Ala-Gly linker (e.g., α6-YFPG366 denotes that the GAG-YFP-GAG cassette was inserted between G366 and V367). Unless otherwise noted, all experiments were conducted with α6-XFP^{A405} and β3-XFP^{P379}.

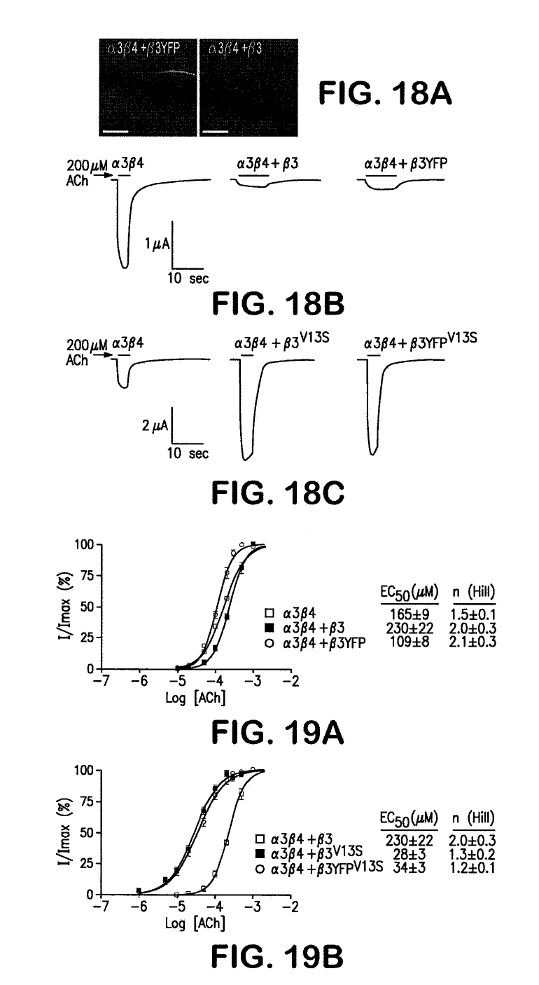

Functional Expression of α6 and β3 Subunits. Despite exhaustive attempts to functionally reconstitute α6* nAChRs in X. laevis oocytes and mammalian tissue culture cells, no robust, reproducible responses were recorded from cells expressing α6, either with untagged subunits or the fluorescent subunits. β3-YFP, however, was well expressed on the plasma membrane of X. laevis oocytes when coexpressed with α3 and β4 subunits to support functional expression (FIG. 18A). As a control for oocyte autofluorescence, oocytes expressing untagged β3 subunits were imaged (FIG. 18A). No fluorescence was detected in this case, indicating that the β3-YFP signal was specific.

β3 subunits do not drastically alter the EC₅₀ for ACh or nicotine when incorporated into nAChRs, but they do profoundly alter single-channel kinetics. Channel burst duration was significantly shortened for nAChRs containing β3 versus those without it, suggesting that β3 reduces the probability of channel opening, Pₒₚₑₙ. Consistent with this, macroscopic voltage-clamped responses from oocytes and mammalian cells expressing β3* receptors were significantly smaller than for non-β3* receptors. To assess the functionality of the β3-YFP construct, the ability of untagged and YFP-labeled β3 subunits to attenuate nicotinic responses was compared. β3 must be coexpressed with other α and β subunits, so α3β4 receptors were chosen for this purpose, because β3 has been well characterized with this receptor combination. When WT, untagged β3 was coexpressed with α3β4 receptors, a significant attenuation of the peak response to 200 μM ACh was found (FIG. 18B). When β3-YFP was tested in this assay, it was also able to attenuate the maximal response in a manner identical to untagged β3 (FIG. 18B). It is possible that, although β3-WT attenuates responses via a gating mechanism on the plasma membrane, YFP-labeled β3 might do so via a different mechanism such as sequestering α3 or β4 subunits inside the cell. To further test the functionality of YFP-labeled β3, the fact that a gain-of-function TM2 mutation in β3 is able to reverse the attenuation of peak responses seen for β3-WT was exploited. It was reasoned that if the YFP label in the M3-M4 loop is not disturbing the function of β3, we should detect the same gain-of-function response for unlabeled and YFP-labeled β3 when they are engineered to express a mutation of this sort. When a Val13′ to Ser mutation (V13S) was introduced into unlabeled β3, not only was a reversal of this attenuation behavior observed, but also a significant increase in the peak response to 200 μM ACh with α3 β4 receptors (FIG. 18C). When β3-YFP^{V13S} was tested in this assay, an identical behavior was observed. Taken together, these data suggested that β3-YFP is fully functional and incorporates into nAChRs in X. laevis oocytes.

To further characterize the β3-YFP construct, concentration-response curves were constructed for α3β4 receptors containing either β3WT or β3-YFP. An EC₅₀ for ACh of 230±22 μM for α3β4β3 receptors we measured, which is slightly higher than for α3β4 (165±9 μM) (FIG. 19A) When β3-YFP was substituted for WT β3, the EC₅₀ was shifted slightly, but acceptably, to 109±8 μM (FIG. 19A). We also noticed that the addition of β3 to α3β4 receptors increased the Hill coefficient from 1.5±0.1 to 2.0±0.3, and this effect was retained when β3-YFP was coexpressed with α3β4 receptors. Likewise, concentration-response relationship curves were also constructed for oocytes expressing β3V13S and β3-YFPV13S. Compared with the EC₅₀ for α3β4β3 (230±22 μM), an EC₅₀ for α3β4β3V13S of 28±3 μM was measured (FIG. 19B). This is consistent with others who have reported an approximate 6-fold reduction in EC₅₀ for the inclusion of β3 with a similar hypersensitive mutation, Val9′Ser (Boorman et al., J Physiol 529:565-77, 2000). It was reasoned that if β3-YFP retained the WT function of β3, then there should be a similar gain-of-function phenotype when it is coexpressed with α3β4. An EC₅₀ for α3β4β3-YFPV13S of 34±3 μM was measured (FIG. 19B), confirming that this construct behaves identically to β3-WT. Collectively, the work in X. laevis oocytes with YFP-labeled β3 subunits suggested that insertion of YFP into the M3-M4 loop did not significantly alter the assembly, subcellular trafficking, or function of this subunit.

Subcellular Localization and Trafficking of α6 and β3 Subunits. To probe the subcellular localization and trafficking of α6* and β3* receptors, a mouse neuroblastoma cell line, N2a, was chosen to transiently express the fluorescent nicotinic receptor subunits. To study the subcellular localization of β3* receptors, β3-YFP were coexpressed with the previously described fluorescently labeled x4 and β2 subunits (Nashmi et al., J Neurosci 23:11554-67, 2003). β3 is able to assemble and function when coexpressed with cc4β2 receptors. When coexpressed with fluorescent α4 or β2 receptors, β3-YFP was localized primarily in the endoplasmic reticulum of live N2a cells. CFP-labeled α4 or β2 subunits was used along with a confocal microscope with spectral imaging capabilities to unambiguously assign YFP and CFP signals to each pixel for the spectral images of the cells. In these experiments, YFP was assigned green, CFP was assigned red, and yellow indicated pixels where β3-YFP was colocalized with either β2-CFP or α4-CFP. It was noted that β3-YFP was completely colocalized with either α4 or β2 in this experiment, suggesting that these subunits are assembled in the same pentameric receptors. To further define the extent of this colocalization, the β3-YFP and α4-CFP or β2-CFP pixel intensity was plotted across a two-dimensional region of interest transecting the cell. It was noted that the YFP and CFP intensity profiles strongly resembled each other, suggesting that these subunits were indeed colocalized and coassembled in intracellular compartments of the cell. With respect to α4β2* receptors, this localization pattern was not an artifact of overexpression, because this was the same pattern we observed previously (Nashmi et al., J Neurosci 23:11554-67, 2003). This was also the expression pattern of endogenous, YFP-labeled α4* receptors in α4-YFP knockin mice (Nashmi et al., J Neurosci 27:8202-18, 2007). This indicated that 1) a large pool of intracellular receptors exists in neurons, and 2) YFP tag does not interfere with the delivery of receptors to the plasma membrane. Thus, the localization pattern observed here for β3 subunits is the expected result if it is assembling with α4β2 receptors.

α6-YFP were expressed along with β2-CFP in N2a cells, and its localization pattern was analyzed as described above for β3. It was also found that α6 was localized in intracellular compartments in the cell, and that it was completely colocalized with β2 subunits. Although this was the first fluorescence imaging reported for α6* receptors, there is other evidence to corroborate these findings. Studies with [3H]epibatidine demonstrate that a significant portion of α6β2 and α6β2β3 receptors are intracellular (˜50 and ˜20%, respectively), although some are delivered to the surface.

To further investigate the subcellular localization and trafficking of α6 and β3 subunits, live, differentiated N2a cells and primary neurons were imaged. N2a cells can be induced to differentiate and undergo neurite outgrowth if serum is withdrawn and an activator of protein kinase A, dibutyryl-cAMP, is added. In other work, α4β2 receptors were localized to dendrites, but not axons, when expressed in primary midbrain neurons. Whether the fluorescent nicotinic receptor subunits were localized to N2a cell processes in a manner analogous to dendrites in primary neurons was addressed next as was the question of whether β3 is localized with other subunits at distal sites such as dendrites. This was an unsolved question, as there was no highaffinity probe (pharmacological or immunological) that could reliably and unambiguously isolate β3* receptors. N2a cells were plated on glass-bottomed dishes, and they were then differentiated for 2 days followed by transfection with various combinations of YFP-labeled and unlabeled nAChR subunits. Cells were also cotransfected with an expression plasmid for soluble CFP to mark total cell morphology. It was found that α4β2 receptors were indeed localized to neuronal processes in differentiated N2a cells, along with abundant expression in the cell soma. When β3-YFP was coexpressed with α4β2, a very similar pattern was observed. It was found that β3 was present even at the most distant elements of neuronal processes. Because this pattern was identical to that of α4β2 in differentiated N2a cells, it was concluded that β3 is likely assembling with α4β2 receptors and that the YFP label in the M3-M4 loop is not disrupting the normal cellular trafficking of α4β2β3 pentamers.

To further characterize the localization of β3* receptors, β3-YFP was coexpressed with α4β2 receptors in primary rat hippocampal neurons. To minimize overexpression artifacts, cells were imaged live only 18 to 24 h after transfection. It was found that β3* receptors were localized very similarly to α4β2 receptors of the studies with primary neurons; uniform localization was noted in the soma, suggestive of endoplasmic reticulum, and dendritic localization and an absence of localization in axons. A high-magnification micrograph demonstrated the dendritic localization of these putative α4β2β3 receptors. In cells coexpressing α4/β2/β3Y with soluble CFP (to mark total cell morphology), β3 subunits did not traffic to a subregion of the cell interior likely to be axons. To more directly determine whether β3* receptors could be localized to axons in these neurons, α4β2β3Y receptors were coexpressed with a CFP-labeled axonal marker, tau. The tau-CFP decorated axons in hippocampal neurons, with proximal (relative to the cell body) portions of the axon being labeled more strongly than distal portions. In all cells examined, the presence of YFP-labeled β3 subunits was noted in these proximal axons but not distal axons. These data in differentiated N2a cells and primary neurons suggested that β3 assembles efficiently with α4β2 receptors, and it is thus cotrafficked and targeted to distal sites in neurons.

Because α6-YFP* receptors did not function, the question of whether this was due to a subtle trafficking defect that could prevent the correct delivery of β6-YFP to the plasma membrane was addressed. Although, α6 fluorescence we could readily detect in the cell body of undifferentiated N2a cells, we wanted to further probe the cellular trafficking of α6* receptors by expressing them in differentiated N2a cells that contain processes. To evaluate the subcellular localization of α6* receptors, α6-YFP was expressed with β2 in differentiated N2a cells. Surprisingly, it was found that α6β2 receptors were trafficked to neuronal processes in a manner analogous to α4β2 and α4β2β3 receptors. To further address this question, α6-YFPβ2 receptors were expressed in rat hippocampal neurons as described for α4β2β3-YFP. A localization pattern was observed for α6-YFP that was very similar to α4β2β3-YFP. These receptors were well expressed in the cell soma, but they were readily detectable in dendrites as well. In experiments with coexpressed soluble CFP and α6-YFPβ2 receptors, α6 subunits were not detected in putative axons (data not shown). In tau-CFP/α6-YFPβ2 coexpression experiments, α6 subunits (similar to α4β2 but not α4β2β3 receptors) were not detected in tau-labeled axons. These data indicated that, although α6* receptors produce little or no agonist-induced conductance in mammalian tissue culture cells, they were expressed well and trafficked similarly compared with α4β2 and β3* receptors.

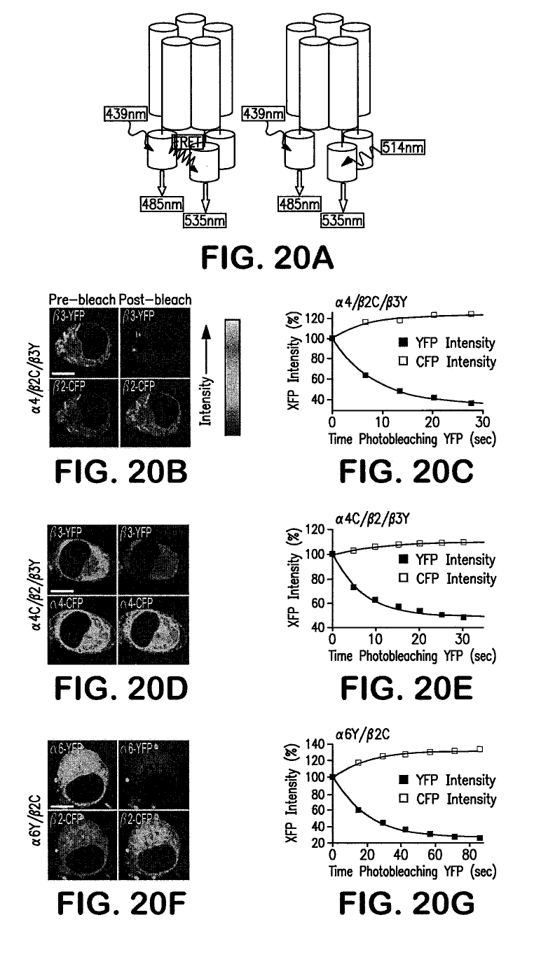

FRET Revealed Assembly of α6 and β3 Subunits into nAChR Pentamers. The fact that α4/β2/β3 and α6/β2 subunits are colocalized in the cell body and cotargeted to processes and dendrites in neurons suggests that they are assembled into pentameric receptors. The question of receptor assembly is often answered by simply measuring agonist-induced conductance increases in cells expressing a subunit combination of interest, or by applying a selective agonist or inhibitor to a pure receptor population of known pharmacological properties. This approach is not applicable, however, for α6* and β3* receptors. α6* receptors do not function well in heterologous expression systems, so it is not straightforward to determine the extent to which free α6 subunits assemble into pentameric receptors. Similarly for β3, although it is functional in oocytes (FIG. 18), there are no pharmacological probes that can be applied to β3* receptors to study their assembly or subunit composition. Others have indirectly measured receptor assembly of nicotinic subunits by using biochemical techniques such as immunoprecipitation and centrifugation or by forcing subunits to assemble by using molecular concatamers. To directly determine whether two nicotinic receptor subunits interact and, possibly, assemble to form pentameric receptors, FRET coupled with the CFP- and YFP-tagged receptors was used. In the context of the nicotinic receptor subunits labeled with YFP or CFP in the M3-M4 loop, only subunits that interact will undergo FRET, because FRET occurs only when donors and acceptors are within 100 Å. Furthermore, it was demonstrated that the efficiency of FRET directly correlates with the number of functional, plasma membrane-localized pentameric receptors. To measure FRET between subunits, the acceptor photobleaching method was used. In this method, CFP dequenching was measured during incremental photodestruction of YFP. CFP was excited at 439 nm, whereas YFP was bleached at 514 nm (FIG. 20A). Because the emission spectra for CFP and YFP overlap significantly, a confocal microscope with spectral imaging capabilities along with a linear unmixing algorithm was used for imaging.

Fluorescent α4 and β2 subunits were functional and underwent robust FRET in mammalian cells, so these subunits were used in the acceptor photobleaching assay with XFP-tagged β3 and α6. β3-YFP was expressed with untagged α4 and β2-CFP in N2a cells, followed by live cell FRET imaging. The whole-cell fluorescence intensity for β3-YFP and β2-CFP was recorded before and after photobleaching of YFP with the 514-nm laser, and we expressed with pseudocolor intensity scaling (FIG. 20B). In this experiment, β2-CFP was clearly dequenched after β3-YFP photodestruction (FIG. 20B), indicating that the two subunits had been undergoing FRET. In a similar experiment, multiple spectral images were recorded at several time points during YFP photodestruction. This revealed a corresponding increase in CFP intensity (FIG. 20C). A reciprocal experiment was also done, where β3-YFP was coexpressed with α4-CFP and untagged β2. A similar dequenching was recorded for α4-CFP after YFP photobleaching (FIG. 20, D and E), and indicated FRET between these subunits as well. Both for β2/β3 and α4/β3 FRET, no difference was found between FRET inside the cell versus FRET at the cell periphery at or near the plasma membrane. These results directly demonstrated that β3 was able to assemble with α4β2 receptors in neuronal cells. This assembly likely occurred in the endoplasmic reticulum, which is consistent with previous findings.

There are many different putative α6* receptor subtypes in brain, including α6β2, α6β2β3, α6α4β2, and α6α4β2β3. To begin to study α6* receptor assembly, FRET was measured between α6-YFP and β2-CFP. In response to YFP bleaching, a robust dequenching of β2-CFP throughout the cell was recorded, indicating FRET between these subunits (FIG. 20, F and G). The pattern of localization and FRET pattern was identical to α4β2β3 receptors.

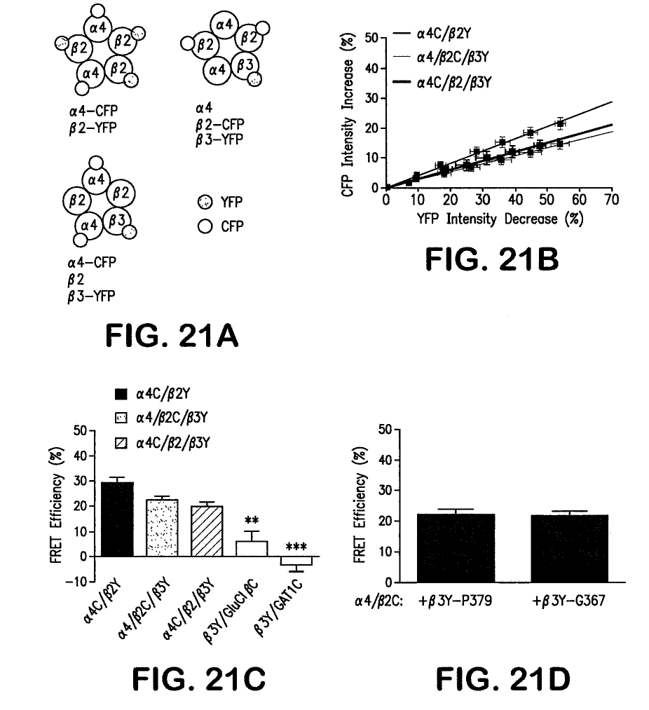

To further quantify FRET between α4/β2 subunits and β3 or α6, we measured FRET E values for various receptor subtypes. α4-CFPβ2-YFP, α4β2-CFPβ3-YFP, and α4-CFPβ2β3-YFP receptors were expressed in N2a cells followed by acceptor photobleaching FRET (FIG. 21A). Spectral images were acquired with 439-nm laser excitation before and during incremental photobleaching of YFP-labeled subunits, followed by extraction of true CFP and YFP image data using linear unmixing. A scatterplot of CFP intensity in response to YFP photobleaching revealed FRET between the subunits in question (FIG. 21B) when the slope of the linear regression line is 0. This slope was used to calculate FRET efficiency values, which were expressed as bar graphs (FIG. 21C). As shown qualitatively in FIG. 20, significant FRET occurred in all nAChR pentamer conditions. A higher FRET E for α4C/β2Y was noted than for β3Y with either β2C or α4C (Y, YFP; C, CFP). To assess the specificity of this measurement, FRET between β3 and a non-nAChR, CFP-labeled protein, mGAT1 was also measured. GAT1 is also a multipass transmembrane protein with a CFP-tag at its C terminus, which faces the cytoplasm. This protein is mainly localized to the endoplasmic reticulum. These two points were important, because it was critical for a specificity probe to have 1) the same membrane topology as the labeled nicotinic receptors, with respect to the attached fluorophore; and 2) the same subcellular localization such that they are capable of interacting with each other. In N2a cells expressing β3-YFP and mGAT1-CFP, no FRET between these proteins was detected (FIG. 21C). In an even more rigorous test, FRET between β3-YFP and another Cys-loop receptor labeled in the M3-M4 loop, the CFP-labeled GluCl β subunit was assessed. FRET between β3 and the GluCl β subunit was significantly smaller (FRET E=6±4%) than for α4 or β2 nAChR subunits (FIG. 21C). Thus, the FRET results between β3 and other labeled nAChR subunits could not be explained by random collision or interaction with unassembled subunits.

Further, the question as to whether subtle changes in the location of the fluorophore within the β3 M3-M4 loop could influence its ability to undergo FRET with another subunit was addressed. FRET E decreases strongly with the distance between fluorophores. It was reasoned that changes in the insertion point of YFP in β3, while holding the position of CFP in β2 constant, might alter FRET between these two subunits. To address this, the FRET E between β2-CFP and two different β3-YFP constructs, β3-YFP^{P379} and β3-YFP^{G367}, which have different insertion points for YFP within the M3-M4 loop was compared. Surprisingly, there was no change in the FRET E for these two subunits (FIG. 21D).

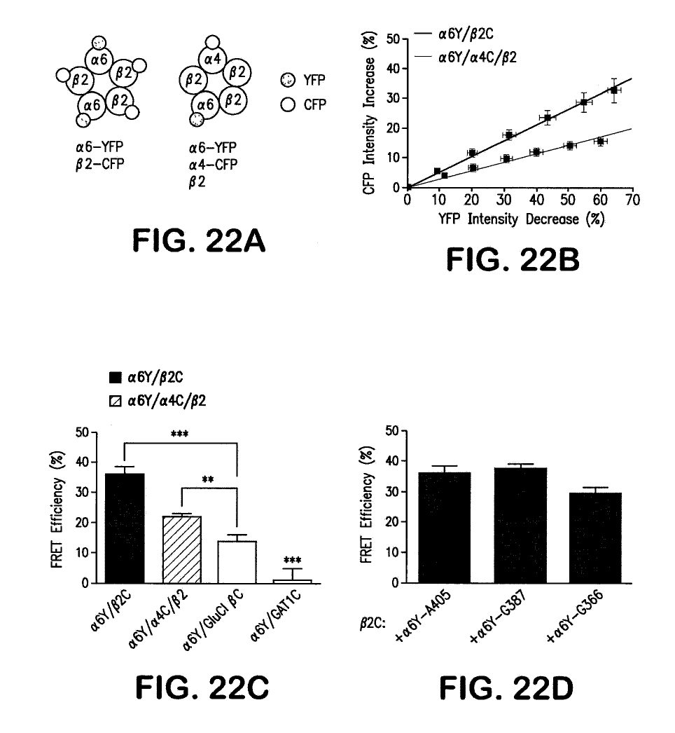

FRET between α6 and α4/β2 subunits was quantitatively measured as well. Either α6Yβ2C or α6Yα4Cβ2 was expressed in N2a cells to measure FRET (FIG. 22A). The latter receptor was studied because recent work indicates that nAChR receptors containing both α6 and α4 1) exist and are functional in mouse brain tissue (Salminen et al., 2007), and 2) are both necessary to form the nAChR subtype with the highest affinity for nicotine yet reported in a functional assay. Acceptor photobleaching FRET experiments revealed robust CFP dequenching in response to YFP photobleach for both of these receptor subtypes, indicating FRET (FIG. 22B). Similar to β3-YFP* receptors, FRET E values were measured for these two subtypes, and a FRET E of 36.0±2.4% for α6Yβ2C and 21.9±1.1% for α6Yα4C, 2 was found (FIG. 22C). The specificity of the FRET measurements for α6 was also assessed by measuring FRET between α6-YFP and mGAT1-CFP as described above for FIG. 21. Similar to β3 and mGAT1, no significant FRET between α6 and mGAT1 was recorded (FIG. 22C). FRET experiments between α6 and the GluCl β subunit, the most rigorous test conducted, yielded a small FRET signal (FRET E=14±2%) (FIG. 22C). Because these subunits presumably do not form functional channels, there may have been a small distortion of the α6 FRET signals that is due to partially assembled receptors. Because this signal was significantly smaller than for all other α6 combinations, FRET between subunits in pentameric receptors remains the most plausible explanation for the energy transfer observed for α6. Finally, FRET was studied between β2-CFP and three α6-YFP constructs (α6-YFP^{A405}, α6-YFP^{G387}, and α6-YFP^{G366}) that differed only in their insertion point for YFP within the M3-M4 loop. No significant difference was found in FRET E between these three α6 constructs (FIG. 22D).

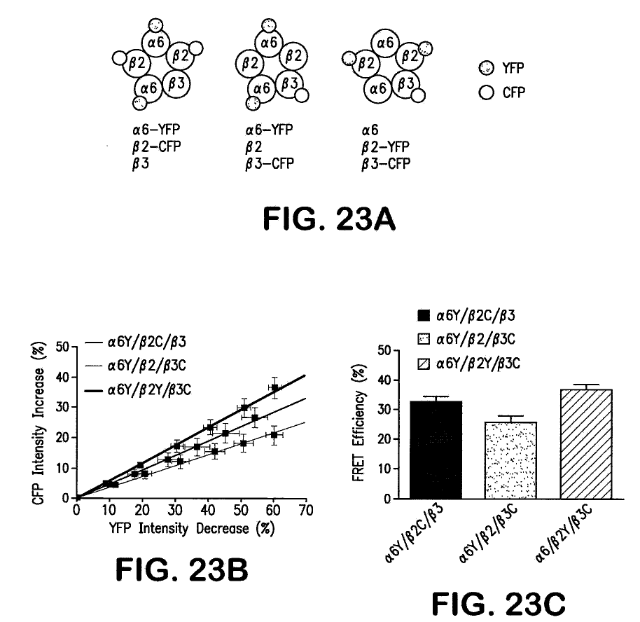

Several results described above suggested that the XFP-labeled β3 and α6 constructs were performing as expected. After confirming that these subunits assemble and traffic normally when expressed independently of each other, these constructs were used together to study α6β2β3 nAChRs. This receptor represents a modest population of the total striatal nAChR pool, and it contributes to nicotine-stimulated dopamine release. α6β2β3 receptors, where one subunit was untagged and the remaining subunits were either YFP- or CFP-tagged (α6Yβ2Cβ3, α6Yβ2β3C, and α6β2Yβ3C), were expressed in N2a cells (FIG. 23A). Robust donor dequenching was measured for all receptor subtypes (FIG. 23B), which was confirmed with FRET E measurements (FIG. 23C). Thus, aside from α6 functional measurements, it was concluded that XFP-labeled α6 and β3 subunits exhibited normal subcellular trafficking and assembly compared with the well-characterized fluorescent α4 and β2 subunits.

α6 and β3 Subunit Stoichiometry Probed with FRET. Fluorescently labeled α6 and β3 were used to probe an important question facing the nicotinic receptor field: subunit stoichiometry. FRET was used herein to address the problem of subunit stoichiometry because FRET occurs only when subunits are directly interacting, and often assembled, with one another.

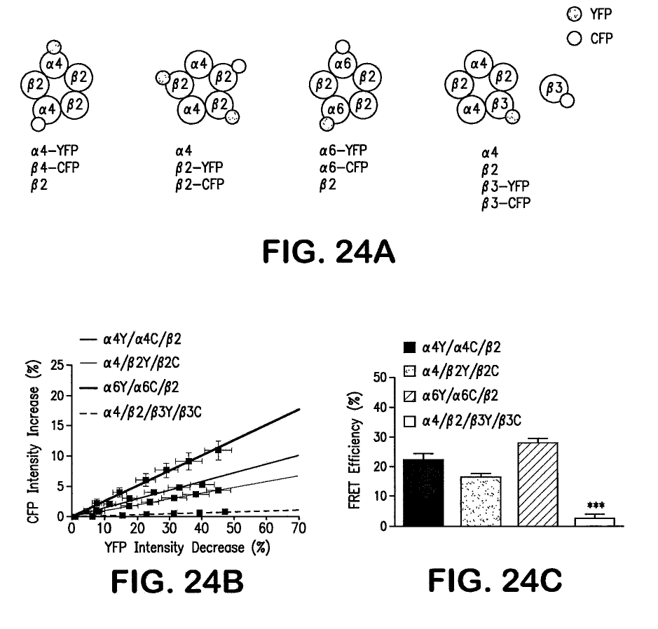

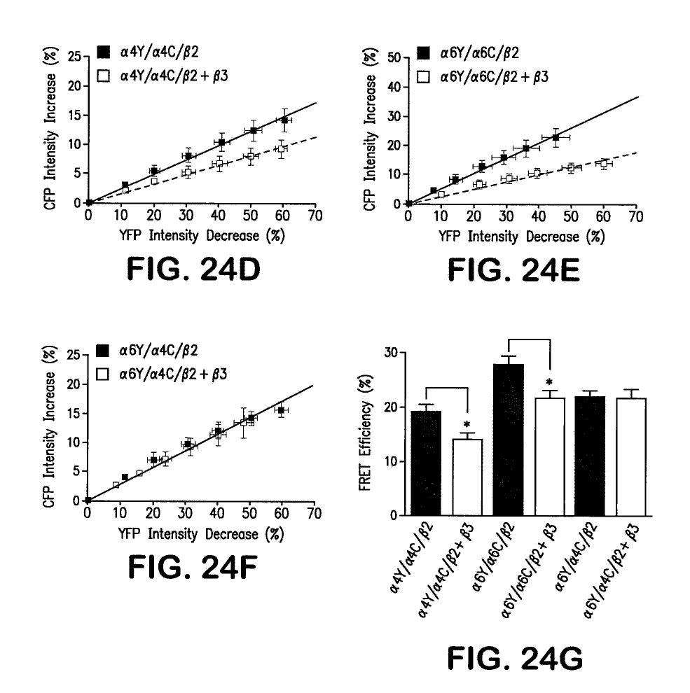

It was previously shown that FRET efficiency correlates directly with functional receptor pentamers. To examine the number of α6 and β3 subunits in a nicotinic receptor pentamer, FRET was first used to examine the stoichiometry of a well studied receptor, namely, α4β2 receptors. It is widely accepted that α4 and β2 subunits assemble to form both high-sensitivity (HS) and low-sensitivity (LS) receptors. Cells often produce a mixture of these two receptors, although they can be induced to express a pure population of one or the other. The subunit stoichiometry of HS receptors is postulated to be (α4)₂(β2)₃, whereas the LS receptors is thought to be (α4)₃(β2)₂. Regardless of the fraction of HS and LS receptors, the fact that all α4β2 receptors presumably contain two or more α4 and two or more β2 subunits was exploited in the studies herein. It was reasoned that when cells express α4-YFP and α4-CFP along with β2 (FIG. 24A), a fraction of the receptors will contain both YFP- and CFP-labeled α4 subunits, and they will therefore be detectable by FRET. Confirming this hypothesis, modest dequenching of α4-CFP was detected upon incremental α4-YFP photobleaching (FIG. 24B). FRET E for α4Yα4Cβ2 receptors was 22.2±2.3% (FIG. 24C). A similar experiment was also conducted with β2, and a modest FRET signal was found (FRET E=16.3±1.7%) between β2-YFP and β2-CFP within the same pentamer (FIG. 24, B and C). This assay was next used to determine whether α6* and β3* receptors have one or more than one α6 or β3 subunit per pentamer. N2a cells expressing either α6Yα6Cβ2 or α4β2β3Yβ3C receptors were analyzed for FRET (FIG. 24A). A strong FRET signal was measured between α6-YFP and α6-CFP in donor dequenching (FIG. 24B), corresponding to a robust FRET E of 27.8±1.7% (FIG. 24C). Thus, these data were the first to directly demonstrate that α6* receptors are capable of containing at least two α6 subunits, similar to other α subunits such as α3 and α4. In contrast to α6, β3 is thought to be an “ancillary subunit”, only able to incorporate into nAChRs with other α and β subunits. Little or no FRET was detected between β3-YFP and β3-CFP (FRET E=2.6±1.3%) (FIG. 24, B and C). This was a specific result, because β3-YFP and β3-CFP were able to FRET with other subunits (FIGS. 7 and 9), thus ruling out the notion that one of these subunits was not able to undergo FRET. These data were the first to directly demonstrate that receptors containing β3 subunits are only able to incorporate a single copy of this subunit. This was interpreted to mean that β3 incorporates into the “accessory” position in a nAChR pentamer, and it likely does not contribute to either of the two α:non-αinterfaces that form the ligand-binding sites.

After confirming via FRET that β3 incorporated into nAChRs at a frequency of one subunit per pentamer, β3 coexpression was used to further probe the subunit stoichiometry of α4* and α6* receptors. β3-WT was coexpresssed with α4-YFP, α4-CFP, and β2 such that β3 was in excess. In this experiment, β3 was incorporated into α4-XFPβ2 receptors and displaced either an α4 or β2 subunit. There was a significant decline in FRET for cells expressing α4Yα4Cβ2β3 receptors versus those expressing α4Yα4Cβ2 (FIG. 24, D and G). This result was interpreted to mean that β3 incorporation has fixed the subunit stoichiometry of FRET-competent receptors to (α4Y)₁(α4C)₁(β2)₂(β3)₁ versus the following mixture of FRET-competent receptors without β3: (α4Y)₂-(α4C)₁(β2)₂, (α4Y)₁(α4C)₂(β2)₂ and (α4Y)₁(α4C)₁(β2)₃. A reduction in FRET for two XFP-labeled α4 subunits (YFP and CFP) versus three is reasonable and expected based on the work of others, and on calculations that predict the relative FRET efficiencies in pentamers with XFP-labeled subunits. Thus, β3 incorporation into nAChR pentamers likely displaces one subunit, and results in a decrease in α4 to α4 FRET for pentamers with a mixed subunit stoichiometry.

To determine whether α6* receptors have a fixed or a mixed subunit stoichiometry, β3 was coexpressed in excess with α6-YFP, α6-CFP, and β2. If α6* receptors only incorporate two α6 subunits, little or no change in FRET was expected to be observed between α6-YFP and α6-CFP because β3 will only displace one unlabeled β2 subunit. However, if α6* receptors exist as a mixture of (α6)₂(β2)₃ and (α6)₃(β2)₂ subtypes similar to α4* receptors, a similar decline in FRET was expected to be observed when β3 is present to induce only the (α6)₂(β2)₂β3 stoichiometry. The latter was the case. A significant decline in the slope of the donor dequenching profile was noted for α6Yα6C* receptors when β3 was present (FIG. 24E) and a decline in the FRET E for α6Yα6Cβ2β3 (21.7±1.4%) versus α6Yα6Cβ2 (27.8±1.7%) (FIG. 24G). Thus, fluorescent α6* receptors behaved identically to α4* receptors, and these results suggested that α6* receptors were capable of forming either of two subunit stoichiometries: (α6)₂(β2)₃ and (α6)₃(β2)₂.

Several groups have reported the existence of α4α6* receptors in brain tissue, and α4α6β2β3* receptors (presumably α4₁α6₁β2₂β3₁) have high affinity for nicotine. To learn about the subunit stoichiometry of α4α6* receptors, α6-YFP and α4-CFP were expressed along with β2 subunits in N2a cells. A modest FRET signal was noted, indicating that these subunits were present in some of the same nicotinic receptor pentamers (FIG. 22, B and C; FIG. 24F). In contrast to the results with α6β2β3 and α4β2β3 receptors, there was no difference between cells transfected with α6Y/α4C/β2 and α6Y/α4C/β2/β3 subunits (FIG. 24, F and G). This showed that addition of excess β3 subunits did not reduce FRET between α6Y and α4C. Thus, this experiment was not informative regarding α4α6β2β3 receptors in N2a cells. For instance, the a: 3 subunit stoichiometry as measured by FRET may not change in the presence of β3.

TIRF Revealed α4*, α6*, and β3* Receptor Plasma Membrane Localization. The plasma membrane localization of nAChRs containing these subunits was probed using TIRF microscopy. For nicotinic receptor subunits fused to fluorescent proteins, TIRF illumination selectively excites only receptors at or very close to the plasma membrane. Live N2a cells expressing α4β2β3-YFP, α6-YFPβ2, and α4-YFPβ2 were imaged. In epifluorescence mode, these receptors exhibited an intracellular, endoplasmic reticulum-like localization identical to the confocal imaging data. In TIRF mode, however, robust plasma membrane fluorescence was noted for all receptor combinations. Surprisingly, α4Yβ2 receptors were found to be localized to distinct, filamentous structures protruding from the cell body. This specific filamentous pattern was seen for 90% of the plasma membrane fluorescence. These structures were reminiscent of filopodia, which are actin-dependent plasma membrane protrusions. To test whether these structures contain actin, a hallmark of filopodia, cells stained with rhodamine-phalloidin, a marker of polymerized actin were imaged. Distinct, actin-containing protrusions were noted. These structures were actin-dependent, because they were destroyed by treatment with latrunculin B, an actin-disrupting agent. These data indicated that, in N2a cells, α4Yβ2 nicotinic receptors were localized to membrane protrusions that strongly resemble filopodia.

Similar to α4Yβ2, live N2a cells expressing either α4β2β3Y or α6Yβ2 were imaged in TIRF mode. Surprisingly, a very different localization pattern was noted compared with α4Yβ2. β3* and α6* receptors were well expressed on the plasma membrane, but there was no evidence of membrane protrusion or filopodia localization for these receptors. Rather, these proteins exhibited a punctate, lattice-like localization pattern on the plasma membrane. This pattern was consistently seen in other cells types such as HEK293, and suggested that β3* or α6* receptors cluster in microdomains distinct from α4β2 receptors. Alternatively, some of these puncta could be clusters of assembled receptors adjacent to the plasma membrane within the 100-nm evanescent wave. Movies to monitor plasma membrane nAChRs were also recorded, and it was noted that although they exhibited localized, stochastic movements, most of these receptor clusters did not travel or translocate to any significant degree. This localization pattern resembled that of the soluble N-ethylmaleimide-sensitive factor attachment protein receptor protein syntaxin1, which was localized to distinct granules or microdomains in the plasma membrane when observed in TIRF. Because soluble N-ethylmaleimide-sensitive factor attachment protein receptor proteins are important regulators of ion channel subcellular trafficking and function in neuronal soma and synaptic terminals (Bezprozvanny et al., 1995), the plasma membrane localization pattern of YFP-syntaxin1A was compared with β3-YFP* and α6-YFP* receptors in N2a cells. A plasma membrane distribution pattern for syntaxin1A was observed that was very similar to β3 and α6 subunits; syntaxin1A was also localized to distinct clusters adjacent to the plasma membrane or microdomains on the plasma membrane.

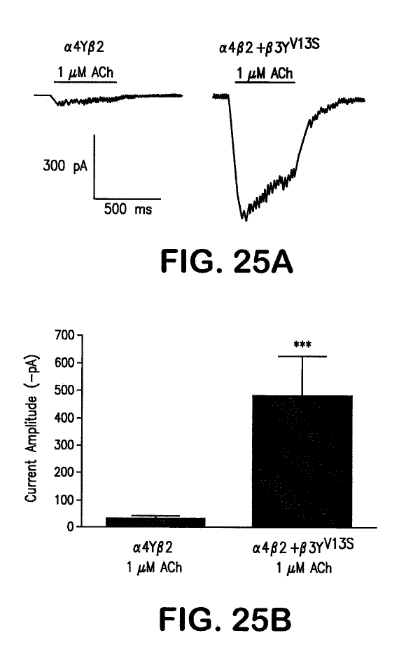

It is possible that the different plasma membrane localization pattern observed for α4Yβ2 versus α4β2β3Y reflects the localization pattern of functional versus nonfunctional nicotinic receptors, respectively. To address this, whole-cell patch-clamp electrophysiology was used to record voltage-clamped responses from functional, fluorescent nAChRs expressed in N2a cells. Because WT (Broadbent et al., 2006) or YFPtagged β3 subunits (FIG. 2B) significantly attenuated nAChR responses, β3-YFPV13S subunits were used to reverse this attenuation. It was reasoned that, if coexpressed and coassembled with α4β2 receptors, β3-YFP^{V13S} subunits should 1) induce the high-sensitivity (α4)₂(β2)₂(β3)₁ subunit stoichiometry; and 2) lower the EC₅₀ for activation of this high-sensitivity form by approximately 1 order of magnitude (FIG. 19B). When voltage-clamped N2a cells expressing α4Yβ2 receptors were stimulated with 1 μM ACh, a dose that induces minimal (2030 pA) responses in other work with HEK293 cells, an identical phenotype was observed (FIG. 24A). Responses to 300 μM ACh were robust (200-400 pA), indicating significant plasma membrane expression of these receptors. However, cells expressing α4β2β3YV13S receptors exhibited robust responses to 1 μM ACh (FIG. 24A), which were significantly larger than the response size for α4β2 receptors (FIG. 25B). This is the expected result if β33-YFPV13S subunits are incorporated into functional nAChRs in N2a cells, and it is consistent with the X. laevis oocyte experiments (FIGS. 18 and 19), and with the work of others. These data confirmed that both α4Yβ2 and α4β2β3Y receptors are functional in N2a cells and that the observed plasma membrane localization pattern for functional α4Yβ2 and α4β2β3Y receptors was significantly different.

Although the invention has been described with reference to the above examples, it will be understood that modifications and variations are encompassed within the spirit and scope of the invention. Accordingly, the invention is limited only by the following claims.

We claim:

1. A transgenic non-human animal comprising a transgene encoding a variant nicotinic acetylcholine receptor (nAChR) subunit, wherein the variant comprises a mutation in the M2 transmembrane region of an nAChR subunit selected from the group consisting of α6, α5, and β2, and wherein further the expression of the variant results in an animal that displays a modified phenotype compared to a wild type animal.

2. The transgenic non-human animal of claim 1, wherein the modified phenotype comprises nicotinic hypersensitivity.

3. The transgenic non-human animal of claim 2, wherein the animal displays psychomotor stimulation by low doses of nicotine, a lack of locomotor sensitization upon repeated activation of nAChRs, a lack of locomotor tolerance upon repeated activation of nAChRs, spontaneous home cage locomotor hyperactivity, or a combination thereof.

4. The transgenic non-human animal of claim 1, wherein the nAChR subunit is the α6 subunit.

5. The transgenic non-human animal of claim 1, wherein the mutation is at any position of the M2 transmembrane region of the nicotinic acetylcholine receptor subunit, and further wherein the mutation renders the receptor hypersensitive.

6. The transgenic non-human animal of claim 1, wherein the mutation is at position 9′ of the M2 transmembrane region of the nicotinic acetylcholine receptor subunit.

7. The transgenic non-human animal of claim 6, wherein the mutation is a leucine to serine mutation.

8. The transgenic non-human animal of claim 6, wherein the mutation is a leucine to alanine mutation.

9. The transgenic non-human animal of claim 1, wherein the mutation is at position 13′ or position 16′ of the M2 transmembrane region of the nicotinic acetylcholine receptor subunit.

10. The transgenic non-human animal of claim 1, wherein the animal is selected from murine, bovine, ovine, porcine, avian, and piscine.

11. The transgenic non-human animal of claim 1, wherein the animal is heterozygous for the variant nicotinic acetylcholine receptor subunit gene.

12. The transgenic non-human animal of claim 1, wherein the variant nAChR subunit comprises a detectable label.

13. The transgenic non-human animal of claim 12, wherein the label is a fluorescent label.

14. A transgenic mouse comprising a transgene encoding a variant nicotinic acetylcholine receptor (nAChR) subunit, wherein the variant comprises a mutation in the M2 transmembrane region of an nAChR subunit selected from the group consisting of α6, α5, and β2, and wherein further the expression of the variant results in a mouse that displays a modified phenotype compared to a wild type mouse.

15. The transgenic mouse of claim 14, wherein the modified phenotype comprises nicotinic hypersensitivity.

16. The transgenic mouse of claim 15, wherein the animal displays psychomotor stimulation by low doses of nicotine, a lack of locomotor sensitization upon repeated activation of nAChRs, a lack of locomotor tolerance upon repeated activation of nAChRs, spontaneous home cage locomotor hyperactivity, or a combination thereof.

17. The transgenic mouse of claim 14, wherein the nAChR subunit is the α6 subunit.

18. The transgenic mouse of claim 14, wherein the mutation is at position 9′ of the M2 transmembrane region of the nicotinic acetylcholine receptor subunit.

19. The transgenic mouse of claim 18, wherein the mutation is a leucine to serine mutation.

20. The transgenic mouse of claim 18, wherein the mutation is a leucine to alanine mutation.

21. The transgenic mouse of claim 14, wherein the mutation is at position 13′ or position 16′ of the M2 transmembrane region of the nicotinic acetylcholine receptor subunit.

22. The transgenic mouse of claim 14, wherein the mouse is heterozygous for the variant nicotinic acetylcholine receptor subunit gene.

23. The transgenic mouse of claim 14, wherein the variant nAChR subunit comprises a detectable label.

24. The transgenic mouse of claim 23, wherein the label is a fluorescent label.

25. A method of generating a transgenic mouse comprising a transgene encoding a variant nicotinic acetylcholine receptor (nAChR) subunit, wherein the method comprises:
a) microinjecting a transgene into a mouse single-cell fertilized egg, wherein the variant comprises a mutation in the M2 transmembrane region of an nAChR subunit selected from the group consisting of α6, α5, and β2;
(b) transferring the microinjected egg cell into a pseudopregnant mouse surrogate; and
(c) identifying mice comprising the transgene from mice born from the surrogate.

26. The method of claim 25, wherein the transgene is contained in a bacterial artificial chromosome.

27. The method of claim 25, wherein the mutation results in an amino acid substitution at position 9′ of the M2 transmembrane region of the α6 nicotinic acetylcholine receptor subunit as compared to a wild-type mouse.

28. The method of claim 27, wherein amino acid substitution is a leucine-to-serine substitution or a leucine-to-alanine substitution at position 9′ in the M2 transmembrane region.

29. A method for screening a candidate agent for the ability to modulate nicotine-mediated behavior in the transgenic animal of claim 1 comprising:
(a) administering to a first transgenic animal of claim 1 a candidate agent, and
(b) comparing nicotine-mediated behavior of the first transgenic animal to nicotine-mediated behavior of a second transgenic animal of claim 1 not administered the candidate agent;
wherein a difference in nicotine-mediated behavior in the first transgenic animal administered the candidate agent compared to the second transgenic animal not administered the candidate agent is indicative of a candidate agent that modifies nicotine-mediated behavior.

30. The method of claim 29, wherein the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.

31. A method of screening for candidate agent that modulates a nicotinic acetylcholine receptor (nAChR) subunit:
(a) administering a candidate agent to a transgenic animal of claim 1; and
(b) determining the effect of the agent upon a cellular or molecular process associated with nicotinic hypersensitivity compared to an effect of the agent administered to a non-transgenic animal, wherein a difference in effect is indicative of an agent that modulates nicotine hypersensitivity.

32. A method of screening for candidate agent that modulates nicotine hypersensitivity comprising:
(a) administering a candidate agent to a transgenic animal of claim 1; and
(b) comparing nicotine-mediated behavior of the first transgenic animal to nicotine-mediated behavior of a non-transgenic littermate animal administered the same dose of the candidate agent, wherein a difference in effect is indicative of an agent that modulates the nicotinic acetylcholine receptor (nAChR) subunit.

33. The method of claim 32, wherein the nicotine-mediated behavior is selected from the group consisting of nicotinic hypersensitivity, psychomotor stimulation by low doses of nicotine, a lack of locomotor sensitization upon repeated activation of nAChRs, a lack of locomotor tolerance upon repeated activation of nAChRs, spontaneous home cage locomotor hyperactivity or a combination thereof.


SEQUENCE LISTING (20 sequences)

SEQ ID NO: 1 - DNA, 15 bp, Mouse
gttctgctttctctc

SEQ ID NO: 2 - DNA, 82 bp, Artificial sequence
  Primer
ttttttaccttccctccgactgtggcgagaaagtgactctttgcatctccgcggccgcggcctggtgatgatggcgggatcg

SEQ ID NO: 3 - DNA, 82 bp, Artificial sequence
  Primer
acgagagatgtggatgggatggtctctgtaatcaccagcaaaaagacagtgcggccgctcagaagaactcgtcaagaaggcg

SEQ ID NO: 4 - DNA, 113 bp, Artificial sequence
  Synthetic construct
ttttttaccttccctccgactgtggcgagaaagtgactctttgcatctccgttctgtcaagcttgactgtctttttgctggtgattacagagaccatcccatccacatctctc

SEQ ID NO: 5 - DNA, 68 bp, Artificial sequence
  Primer
agcctcacaagacctgacagctcactgggcatcagtgaagtgcaccctgcagggacgtcaggtggcac

SEQ ID NO: 6 - DNA, 68 bp, Artificial sequence
  Primer
tgagagagtggcactgagagccaagaagacccgtaggaagcctgtcctgcagggtctgacgctcagtg

SEQ ID NO: 7 - DNA, 19 bp, Artificial sequence
  Primer
ctccgttctgtcaagcttg

SEQ ID NO: 8 - DNA, 21 bp, Artificial sequence
  Primer
acgagtgctctgaattctctg

SEQ ID NO: 9 - DNA, 21 bp, Artificial sequence
  Primer
gctcatgagacaataaccctg

SEQ ID NO: 10 - DNA, 23 bp, Artificial sequence
  Primer
cagtcttggaagcaacatccagc

SEQ ID NO: 11 - DNA, 21 bp, Artificial sequence
  Primer
ctgctgctcatcaccgagatc

SEQ ID NO: 12 - DNA, 21 bp, Artificial sequence
  Primer
cagatgtcacccaagatgccg

SEQ ID NO: 13 - DNA, 18 bp, Artificial sequence
  Primer
gagcgctgctgacacttg

SEQ ID NO: 14 - DNA, 18 bp, Artificial sequence
  Primer
ccccttgtagcacctagc

SEQ ID NO: 15 - DNA, 21 bp, Artificial sequence
  Primer
cagatgtcacccaagatgccg

SEQ ID NO: 16 - DNA, 22 bp, Artificial sequence
  Primer
gttttacaccatcaacctcatc

SEQ ID NO: 17 - DNA, 21 bp, Artificial sequence
  Primer
ttaggagtctgtgtacttggc

SEQ ID NO: 18 - DNA, 15 bp, Artificial sequence
  Synthetic construct
gttctgtcaagcttg

SEQ ID NO: 19 - protein, 136 aa, Mouse
RTPATHTMPKWVKTIFLQAFPSILMMRKPLDKTKEAGGVKDPKSHTKRPAKVKFTHRGESKLLKECHHCQKSSDIAPGKRRSSQQPARWVAENSEHSSDVEDVIESVQFIAENMKSHNETNEVEDDWKYMAMVVDR

SEQ ID NO: 20 - protein, 108 aa, Mouse
VHHRSSSTYHPMAPWVKRLFLEKLPRWLCMKDPRDRFSFPDGTESKGTVRGKFPGKKKQTPTSDGERVLVAFLEKASESIRYISRHVKKEHFISQVVQDWKFVAQVLD